(19) United States
(12) Patent Application Publication
(10) Pub. No.: US 2013/0096103 A1
(43) Pub. Date: Apr. 18, 2013

(54) IMIDAZOQUINOLINE COMPOUNDS

(71) Applicants: Nicholas VALIANTE, Fremont, CA (US); Feng Xu, Walnut Creek, CA (US); Xiaodong Lin, Orinda, CA (US); Daniel Chu, Santa Clara, CA (US); Xiaojing Michael Wang, Livermore, CA (US)

(72) Inventors: Nicholas VALIANTE, Fremont, CA (US); Feng Xu, Walnut Creek, CA (US); Xiaodong Lin, Orinda, CA (US); Daniel Chu, Santa Clara, CA (US); Xiaojing Michael Wang, Livermore, CA (US)

(73) Assignee: NOVARTIS VACCINES & DIAGNOSTICS, INC., Emeryville, CA (US)

(21) Appl. No.: 13/527,537

(22) Filed: Jun. 19, 2012

Related U.S. Application Data
(63) Continuation of application No. 13/108,937, filed on May 16, 2011, now abandoned.
Application No. 13/108,937 is a continuation of application No. 11/662,984, filed on Oct. 25, 2007, now abandoned.
(60) Provisional application No. 60/637,107, filed on Dec. 16, 2004.
(60) Provisional application No. 60/609,586, filed on Sep. 14, 2004.

(51) Int. Cl. C07D 471/14

(52) U.S. Cl. CPC C07D 471/14
USPC 514/210.21; 546/82; 514/293

(57) ABSTRACT

The invention provides novel compositions comprising imidazoquinoline compounds. Also provided are methods of administering the compositions in an effective amount to enhance the immune response of a subject. Further provided are novel compositions and methods of administering the compositions in combination with (an)other agent(s).

### CROSS-REFERENCE TO RELATED APPLICATIONS

This application is a Continuation of U.S. patent application Ser. No. 13/108,937, with a filing date of May 16, 2011, which is a Continuation of U.S. patent application Ser. No. 11/662,984 filed on Oct. 25, 2007, which is the National Stage of International Patent Application of PCT/US2005/032721, filed Sep. 14, 2005, which claims priority to U.S. Provisional Patent Applications Nos. 60/637,107, filed Dec. 16, 2004 and 60/609,586, filed Sep. 14, 2004, all of which are hereby incorporated by reference in their entireties.

### FIELD OF THE INVENTION

The present invention generally relates to small molecule immune potentiators (SMIPs) such as novel imidazoquinoline compounds that are capable of stimulating or modulating an immune response in a subject. The invention also relates to novel combinations of antigens with immune potentiators that may be used in vaccine therapies. In some embodiments, the compounds can be used as immunotherapeutic agents for proliferative diseases, infectious diseases, autoimmune diseases, allergies, and/or asthma.

### BACKGROUND OF THE INVENTION

With the number and diversity of diseases burgeoning and respective therapeutic treatments receding, a new therapeutic approach is needed. Such an approach should be focused less on targeting specific substrates in the disease state and more on bolstering the immune response to the disease. Since the discovery of penicillin, which targets bacteria-specific cell walls conveniently absent in man, the model of modern medicine has been to eliminate substrates in the disease state, while leaving the host system unaffected. Unfortunately, few therapies have ever reached that pinnacle and fewer still remain effective in the face of resistance mutations. Applied to cancer, upregulated kinases have been the targets of therapeutic development. Unfortunately, the only recent therapeutic agent to hit the bull's-eye is Gleevec, and likely not solely because of its kinase inhibitory activity. Borg et al. J. Clin. Invest. 114:379-388 (2004).

There are numerous benefits to (or detriments to not) potentiating an immune response instead of, or in addition to, disease substrate inhibition. One advantage is that substrates in the disease and host are commonly shared, although possibly upregulated in the disease state. For example, cancer drugs targeting kinases may be cytotoxic and may destroy cellular machinery in the host in addition to the cancer cells. Subsequently, the maximum tolerated doses (MTDs) necessary for treatment efficacy may result in undesirable side effects and even weak the immune response in the patient. Such side effects may require cessation of treatment. Conversely, as seen with Gleevec, the dual action of inhibiting bcr-abl, while stimulating an immune response, likely contributes to its efficacy and tolerability, particularly because NK cells, which are stimulated by administration of Gleevec, independently play a role in tumor recession. This synergistic approach to cancer regression is extremely effective. Alternatively, cytotoxics that suppress the immune system may independently contribute to the disease state since they may inhibit separate pathways that may be involved in recovery.

Another advantage to immune potentiation is that it provides a platform less easily bypassed by resistance mutations. Where therapeutic targets are so polarized and specific (which may be necessary in order to avoid targeting host cells), such as a particular substrate in a viral replicon or a kinase in a cancer cell line, a single point mutation in the disease state may render it unaffected by a drug resulting in even harsher strains of the disease in future generations.

Novel methods and mechanisms for treating patients having diseases resistant to, or inadequately treated by, conventional approaches utilizing agents targeting specific immune response mechanisms in the body is needed.

U.S. Pat. Nos. 4,547,511 and 4,738,971 disclose compounds for the treatment of disorders responsive to agents that enhance cell-mediated immunity. The compounds disclosed in these patents have the general formula (a):

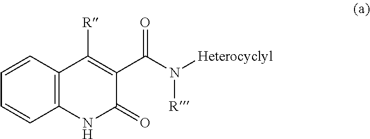

Neither patent, however, contemplates using compounds of formula (a) in combination with antigens.

Immunostimulatory oligonucleotides and polynucleotides are described in WO 98/55495 and WO 98/16247. Published U.S. Patent Application No. 2002/0164341 describes adjuvants including an unmethylated CpG dinucleotide (CpG ODN) and a non-nucleic acid adjuvant. U.S. Patent Application No. 2002/0197269 describes compositions comprising an antigen, an antigenic CpG-ODN, and a polycationic polymer. Each of these references is hereby incorporated by reference in its entirety and for all purposes as if fully set forth herein.

Issued U.S. Pat. Nos. 4,689,338, 5,389,640, 5,268,376, 4,929,624, 5,266,575, 5,352,784, 5,494,916, 5,482,936, 5,346,905, 5,395,937, 5,238,944, 5,525,612, 6,083,505, and 6,110,929, and WO 99/29693 disclose imidazoquinoline compounds of the general structure (b) for use as “immune response modifiers”:

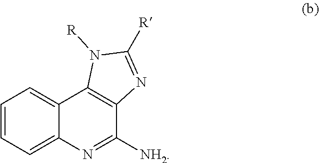

Each of these references is hereby incorporated by reference in its entirety and for all purposes as if fully set forth herein.

WO 03/097641 discloses the use of certain imidazoquinolines and salts thereof for the treatment of certain protein kinase dependent diseases and for the manufacture of pharmaceutical preparations for the treatment of diseases.

Immune response to certain antigens that are otherwise weakly antigenic can be enhanced through the use of immune potentiators, known as vaccine adjuvants. Such adjuvants potentiate the immune response to specific antigens and are, therefore, the subject of considerable interest and study within the medical community.

Research has resulted in the development of vaccines possessing antigenic epitopes that were previously impossible to produce. For example, currently available vaccine candidates include synthetic peptides mimicking streptococcal, gonococcal, and malarial antigens. These purified antigens are generally weak antigens that require adjuvants in order to evoke protective immunity. Unfortunately, conventional vaccine adjuvants possess a number of drawbacks that limit their overall use and effectiveness. For example, mineral oil is known to produce tissue irritation and to be potentially oncogenic. Alum, the only approved adjuvant in the United States, also induces granulomas at the inoculation site and, furthermore, does not effectively induce cell-mediated immunity. Moreover, many of the adjuvants currently available have limited utility because they include components that are not metabolized by humans. Additionally, most adjuvants are difficult to prepare and may require time-consuming procedures and, in some cases, the use of elaborate and expensive equipment to formulate a vaccine and adjuvant system.

Immunological adjuvants are described in “Current Status of Immunological Adjuvants”, Ann. Rev. Immunol., 1986, 4, pp. 369-388, and “Recent Advances in Vaccine Adjuvants and Delivery Systems” by Derek T O'Hagan and Nicholas M. Valiante. See also U.S. Pat. Nos. 4,806,352; 5,026,543; and 5,026,546 for disclosures of various vaccine adjuvants appearing in the patent literature. Each of these references is hereby incorporated by reference in its entirety and for all purposes as if fully set forth herein.

Efforts have been made to identify new immune modulators for use as adjuvants for vaccines and immunotherapies that would overcome the drawbacks and deficiencies of conventional immune modulators. In particular, an adjuvant formulation that elicits potent cell-mediated and humoral immune responses to a wide range of antigens in humans and domestic animals, but lacking the side effects of conventional adjuvants and other immune modulators, would be highly desirable. This need could be met by small molecule immune potentiators (SMIPs) because the small molecule platform provides diverse compounds for the selective manipulation of the immune response, necessary for increasing the therapeutic index immune modulators.

Novel sole-acting agents with varied capacities for altering levels and/or profiles of cytokine production in human immune cells are needed. Compounds with structural disparities will often elicit a desired response through a different mechanism of action, or with greater specificity to a target, such as a dendritic cell, modulating potency and lowering side effects when administered to a patient.

The immunosuppressive effect of cytostatic substances has rendered them useful in the therapy of autoimmune diseases such as multiple sclerosis, psoriasis and certain rheumatic diseases. Unfortunately, their beneficial effect has to be weighed against serious side effects that necessitate dosages that are too low. Furthermore, interruption of the treatment may be required.

Agents and/or combinations of active substances that result in significantly improved cytostatic or cytotoxic effects compared to conventional cytostatics, e.g., vincristin, methotrexate, cisplatin, etc., are needed. With such agents and combinations, chemotherapies may be offered that combine increasing efficiency with a large reduction of side effects and therapeutic doses. Such agents and combination therapies may thus increase the therapeutic efficiency of known cytostatic drugs. In some embodiments, the compounds of the invention are used in combination with compounds that provide significantly improved cytostatic or cytotoxic effect compared to conventional cytostatic agents when administered alone. Additionally, cell lines that are insensitive to conventional chemotherapeutic treatment may also be susceptible to chemotherapy using combinations of active substances.

The current invention provides individual therapeutic and prophylactic agents for treatment of disease states characterized by other immune deficiencies, abnormalities, or infections including autoimmune diseases and viral and bacterial infections responsive to compounds with the capacity to modulate cytokines and/or TNF-α, such as multiple sclerosis, Crohn's disease, HIV, HSV, and HCV, among others.

Therapeutics that serve to augment natural host defenses against viral and bacterial infections, or against tumor induction and progression, with reduced cytotoxicity, are needed. The present invention provides such therapeutic agents, and further provides other related advantages.

### BRIEF SUMMARY OF THE INVENTION

The instant invention provides novel immune potentiators, immunogenic compositions, novel compounds and pharmaceutical compositions, and novel methods of administering a vaccine, by administering small molecule immune potentiators alone or in combination with antigens and/or other agents. The invention further provides novel compounds and pharmaceutical compositions, for use in the treatment of cancer, precancerous lesions, autoimmune diseases, infectious diseases, allergies, and asthma. The invention further provides the use of the compounds of the invention in the manufacture of medicaments for use in the treatment of cancer, precancerous lesion, autoimmune diseases, allergies, and asthma.

The imidazoquinoline compounds used in the methods and compositions of the invention are inexpensive to produce and easy to administer. They have potential for finer specificity compared to existing immunostimulants, thus providing improved efficacy and safety profiles.

As adjuvants, the imidazoquinoline compounds may be combined with numerous antigens and delivery systems to form a final vaccine product.

As immunotherapeutics, the imidazoquinoline compounds are used alone or in combination with other therapies (e.g., anti-virals, anti-bacterials, other immune modulators or in therapeutic vaccine antigens) for treatment of chronic infections such as those caused by the human immunodeficiency virus (HIV), the hepatitis C virus (HCV), the hepatitis B virus (HBV), the herpes simplex virus (HSV), and H. pylori, as well as medicaments for the reduction of tumor growth or modulation of abnormal cellular proliferation associated with diseases such as actinic keratosis, atypical or dysplastic nevi, or premalignant lentigos.

The imidazoquinoline compounds of the present invention also target substrates in the disease state, such as, for example particular kinases including EGFr, c-Kit, bFGF, Kdr, CHK1, CDK, cdc-2, Akt, PDGF, PI3K, VEGF, PKA, PKB, src, c-Met, Abl, Ras, RAF, and MEK, among others.

As immunotherapeutics, the imidazoquinoline compounds may also be used for the treatment of cancer either alone or in combination with other anti-cancer therapies (e.g., chemotherapeutic agents, (monoclonal antibodies) mAbs or other immune potentiators). In addition, certain imidazoquinolines with the capacity to induce Type 1 cytokines (e.g., IL-12, TNF-α or IFN's) may be used for the treatment of allergies and/or asthma due to their capacity to steer the immune response towards more benign sequelae. The imidazoquinoline compounds may be used, for example, for the treatment of bacillus Calmette-Guerin (BCG), cholera, plague, typhoid, hepatitis B infection, influenza, inactivated polio, rabies, measles, mumps, rubella, oral polio, yellow fever, tetanus, diphtheria, hemophilus influenzae b, meningococcus infection, and pneumococcus infection. The imidazoquinoline compounds may be used in an anti cell proliferative effective amount for the treatment of cancer. The imidazoquinoline compounds may also be used in anti-Th2/Type2 cytokine amount for the deviation of allergic/asthmatic immune responses.

In some embodiments, methods of treating cancer and/or precancerous lesions are provided. In such embodiments, one or more known anticancer agent is combined with one or more imidazoquinoline compound to reduce tumor growth in a subject. A number of suitable anticancer agents are contemplated for use in the methods of the present invention and are described more thoroughly in the following detailed description.

In accordance with another embodiment, there is provided a method of inhibiting tumor cell growth in a subject. The method includes administering to a subject an effective dose of a combination comprising at least one SMIP and a monoclonal antibody (mAb). The combination is more effective at inhibiting such cell growth than when the mAb is administered by itself. In some embodiments of the methods of treating cancer with the combination, an additional SMIP compound and/or mAb, is administered to the subject.

In some embodiments of the methods and composition of the invention, the imidazoquinoline compound is selected from one or more of those in the following list:
N2-methyl-1-(2-methylpropyl)-1H-imidazo[4,5-c]quinoline-2,4-diamine;
N2,N2-dimethyl-1-(2-methylpropyl)-1H-imidazo[4,5-c]quinoline-2,4-diamine;
N2-ethyl-N2-methyl-1-(2-methylpropyl)-1H-imidazo[4,5-c]quinoline-2,4-diamine;
N2-methyl-1-(2-methylpropyl)-N2-propyl-1H-imidazo[4,5-c]quinoline-2,4-diamine;
1-(2-methylpropyl)-N2-propyl-1H-imidazo[4,5-c]quinoline-2,4-diamine;
N2-butyl-1-(2-methylpropyl)-1H-imidazo[4,5-c]quinoline-2,4-diamine;
N2-butyl-N2-methyl-1-(2-methylpropyl)-1H-imidazo[4,5-c]quinoline-2,4-diamine;
N2-methyl-1-(2-methylpropyl)-N2-pentyl-1H-imidazo[4,5-c]quinoline-2,4-diamine;
N2-methyl-1-(2-methylpropyl)-N2-prop-2-enyl-1H-imidazo[4,5-c]quinoline-2,4-diamine;
1-(2-methylpropyl)-2-[(phenylmethyl)thio]-1H-imidazo[4,5-c]quinolin-4-amine;
1-(2-methylpropyl)-2-(propylthio)-1H-imidazo[4,5-c]quinolin-4-amine; 2-[[4-amino-1-(2-methylpropyl)-1H-imidazo[4,5-c]quinolin-2-yl](methyl)amino]ethanol;
2-[[4-amino-1-(2-methylpropyl)-1H-imidazo[4,5-c]quinolin-2-yl](methyl)amino]ethyl acetate;
4-amino-1-(2-methylpropyl)-1,3-dihydro-2H-imidazo[4,5-c]quinolin-2-one;
N2-butyl-1-(2-methylpropyl)-N4,N4-bis(phenylmethyl)-1H-imidazo[4,5-c]quinoline-2,4-diamine;
N2-butyl-N2-methyl-1-(2-methylpropyl)-N4,N4-bis(phenylmethyl)-1H-imidazo[4,5-c]quinoline-2,4-diamine;
N2-methyl-1-(2-methylpropyl)-N4,N4-bis(phenylmethyl)-1H-imidazo[4,5-c]quinoline-2,4-diamine;
N2,N2-dimethyl-1-(2-methylpropyl)-N4,N4-bis(phenylmethyl)-1H-imidazo[4,5-c]quinoline-2,4-diamine;
1-{4-amino-2-[methyl(propyl)amino]-1H-imidazo[4,5-c]quinolin-1-yl}-2-methylpropan-2-ol;
1-[4-amino-2-(propylamino)-1H-imidazo[4,5-c]quinolin-1-yl]-2-methylpropan-2-ol;
N4,N4-dibenzyl-1-(2-methoxy-2-methylpropyl)-N2-propyl-1H-imidazo[4,5-c]quinoline-2,4-diamine;
1-(4-Amino-2-propylsulfanyl-imidazo[4,5-c]quinolin-1-yl)-2-methyl-propan-2-ol;
1-(4-Amino-2-azetidin-1-yl-imidazo[4,5-c]quinolin-1-yl)-2-methyl-propan-2-ol;
1-(4-Amino-2-pyrrolidin-1-yl-imidazo[4,5-c]quinolin-1-yl)-2-methyl-propan-2-ol;
1-(4-Amino-2-cyclopropylsulfanyl-imidazo[4,5-c]quinolin-1-yl)-2-methyl-propan-2-ol; or
1-(4-Amino-2-isobutylsulfanyl-imidazo[4,5-c]quinolin-1-yl)-2-methyl-propan-2-ol.

Additional embodiments, methods and compositions contemplated to be useful in the instant invention are disclosed in U.S. Ser. No. 10/814,480, 10/762,873, 60/582,654, 10/405,495, and 10/748,071 which are each hereby incorporated by reference in their entireties and for all purposes as if set forth fully herein.

Methods of manufacturing compounds and compositions described herein are provided and contemplated to fall within the scope of the invention as is the use of the imidazoquinolines in methods for manufacturing medicaments for use in the methods of the invention.

In each of the embodiments of the invention, compounds, such as those of Formula I, can be used in the manufacture of a medicament for enhancing the immune response to an antigen.

Other embodiments provide the use of the compounds of the invention, in the manufacture of medicament for immune stimulation, and another agent, such as an antigen, for simultaneous separate or sequential administration. In another more particular embodiment the use is for treating or preventing a bacterial or viral infection. In another embodiment the use is for treating cancer. In another embodiment the use is for preventing influenza infection and the antigen is hemagglutinin and/or neuraminidase surface protein(s).

Other embodiments provide a pharmaceutical preparation or system, comprising (a) a compound (such as a compound of formula I) according to any of the aspects/embodiments described herein; and (b) an antigen, wherein the first and second agents are either in admixture or are separate compositions. In a more particular embodiment the second agent is hemagglutinin and/or neuraminidase surface protein. More specifically, the agents are for simultaneous separate or sequential administration. In another more particular embodiment the use is for preventing an infection. In another embodiment the use is for treating cancer.

Further embodiments of the invention include those described in the detailed description.

### BRIEF DESCRIPTION OF THE FIGURES

FIG. 1 shows TLR7 (FIG. 1A) and TLR8 (FIG. 1B) dependence of SMIPs according to the present invention.

FIG. 2A shows multi-cytokine assays for SMIP potency on myelomonocytic cell line, THP-1.

FIG. 2B shows multi-cytokine assays for SMIP potency on human PBMC.

FIG. 2C shows multi-cytokine assays for SMIP potency on murine splenocytes.

FIG. 3 shows ranking of SMIP potency in varying cell lines.

FIG. 4 shows in vivo adjuvant activity of the compounds of Example 11 and Example 19, specifically, the anti-gp120-specific serum IgG2a geometric mean titers from 2 weeks post-second serum of BALB/c mice immunized 2× with HIV gp120 formulated in MF59 +/− the indicated SMIPs (FIG. 4A); the IgG1 geometric mean titers from 2 weeks post-second serum (FIG. 4B), and ex vivo anti-gp120-specific T cell responses from spleens harvested from immunized mice (FIG. 4C).

### DETAILED DESCRIPTION OF THE INVENTION

Applicants have discovered methods of stimulating cytokine activity in cells and immunotherapeutics and/or vaccine adjuvants, that will provide effective treatments for disorders such as those described herein and those apparent to one skilled in the art.

In one embodiment, the invention provides a compound of formula (I):

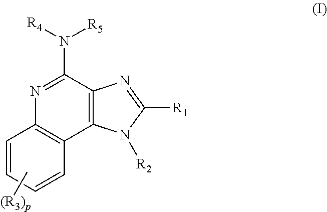

wherein:

R₁ is —NR₆R₇, —C(O)R₅, —C(O)OR₈, —C(O)NR₆R₇, —(CH₂)ₘCH≡CH(CH₂)ₙR₉, —(CH₂)ₘC≡C(CH₂)ₙR₉, or —S(O)_{q}R₁₀;

R₂ is H, C₁₋₆ alkyl, substituted C₁₋₆ alkyl, —(CH₂)ₘCH═CH(CH₂)ₙR₉, —(CH₂)ₘC≡C(CH₂)ₙR₉, —C(O)R₈, —C(O)OR₈, —C(O)NR₆R₇, or —S(O)_{q}R₁₀;

each R₃ is independently H, C₁₋₆ alkyl, substituted C₁₋₆ alkyl, C₁₋₆ alkoxy, halogen, trihalomethyl, —NR₆R₇, —C(O)R₈, —C(O)OR₈, or —C(O)NR₆R₇;

R₄ and R₅ are each independently H, C₁₋₆ alkyl, C₆₋₁₀ aryl-C₁₋₆ alkyl, or a protecting group;

each R₆ and R₇ is independently H, C₁₋₆ alkyl, substituted C₁₋₆ alkyl, C₁₋₆ alkoxy, C₁₋₆ alkoxy-C₁₋₆ alkyl, C₆₋₁₀ aryl, C₆₋₁₀ aryl-C₁₋₆ alkyl, C₆₋₁₀ aryloxy-C₁₋₆ alkyl, —(CH₂)ₘCH═CH(CH₂)ₙR₉, or —(CH₂)ₘC≡C(CH₂)ₙR₉; or

R₆ and R₇ are taken together to form a substituted or unsubstituted heterocyclyl group;

each R₈ is independently H, C₁₋₆ alkyl, or substituted C₁₋₆ alkyl;

each R₉ is independently H, C₁₋₆ alkyl, substituted C₁₋₆ alkyl, C₂₋₆ alkenyl, C₆₋₁₀ aryl, —CO₂H, —C(O)O—C₁₋₆ alkyl or halo;

each R₁₀ is independently C₁₋₆ alkyl, substituted C₁₋₆ alkyl, C₂₋₆ alkenyl, C₆₋₁₀ aryl, C₆₋₁₀ aryl-C₁₋₆ alkyl, trihalomethyl, or —NR₆R₇;

each m and n is independently 0, 1, 2, or 3;

p is 0, 1, 2, or 3; and

each q is independently 0, 1, or 2; or

a pharmaceutically acceptable salt thereof, a tautomer thereof, or a pharmaceutically acceptable salt of the tautomer.

In some embodiments, if q within R₁ is 0 and R₁₀ within R₁ is methyl such as if R₁ is —S-Me, then R₂ is not isobutyl.

In another embodiment, R₄ and R₅ are each H. In still other embodiments, R₄ and R₅ are each H, and p is 0.

In another embodiment, R₄ and R₅ are each H and R₁ is —NR₆R₇, —S(O)_{q}R₁₀, —C(O)NR₆R₇, —(CH₂)ₘCH═CH(CH₂)ₙR₉, or —(CH₂)ₘC≡C(CH₂)ₙR₉.

In another embodiment, R₄ and R₅ are each H and R₁ is —NR₆R₇ where R₆ and R₇ are independently H, unsubstituted C₁₋₆ alkyl or —(CH₂)ₘCH═CH(CH₂)ₙR₉.

In another embodiment, R₁ is —NR₆R₇. In some such embodiments thereof, R₆ and R₇ within R₁ are independently selected from H, C₁₋₆ alkyl or —(CH₂)ₘCH═CH(CH₂)ₙR₉. In other embodiments thereof, the C₁₋₆ alkyl of the R₆ and/or R₇ groups of the R₁—NR₆R₇ is/are independently selected from methyl, ethyl, propyl, n-butyl, or n-pentyl. In some such embodiments, R₆ and R₇ are propyl and methyl respectively. In other embodiments, R₆ is methyl and R₇ is —(CH₂)ₘCH═CH(CH₂)ₙR₉ where m is 1, n is 0, and R₉ is H.

In another embodiment, R₁ is —S(O)_{q}R₁₀. In some such embodiment thereof, q and R₁₀, within R₁, are O and C₁₋₆ alkyl, respectively such that R₁ is —SR₁₀ where the R₁₀ of the —SR₁₀ is C₁₋₆ alkyl such that R₁ is —S—C₁₋₆ alkyl. In another embodiment, the C₁₋₆ alkyl is ethyl such that R₁ is —S-Ethyl. In another embodiment, the C₁₋₆ alkyl is —CH₂CH₂CH₃ such that R₁ is —SCH₂CH₂CH₃. In another embodiment, the C₁₋₆ alkyl is —CH(CH₃)₂ such that R₁ is —S—CH(CH₃)₂. In other embodiment, q and R₁₀, within R₁, are 0 and C₆₋₁₀ aryl-C₁₋₆ alkyl, respectively such that R₁ is —S—(C₆₋₁₀ aryl-C₁₋₆ alkyl). In some such embodiments, R₁₀ is benzyl such that R₁ is —S—CH₂Ph.

In other embodiments, R₁ is —C(O)NR₆R₇.

In still other embodiments, R₁ is —(CH₂)ₘCH═CH(CH₂)ₙR₉.

In yet other embodiments, R₁ is —(CH₂)ₘC≡C(CH₂)ₙR₉.

In another embodiment, R₂ is C₁₋₆ alkyl. In some such embodiments, R₂ is isobutyl.

In other embodiments, m is 1, n is 0, and R₉ is H.

In still other embodiments, p is 0.

In yet other embodiments, R₂ is substituted C₁₋₆ alkyl. In some such embodiments thereof, R₂ is —CH₂C(CH₃)₂(OH). In another embodiment, R₂ is —CH₂C(CH₃)₂NH—SO₂CH₃.

In other embodiments, R₁ is —S-cyclopropyl, —S—CH₂CH(CH₃)₂, or —S—CH₂CH₂CH₃.

In other embodiments, R₁ is —S—C₃₋₆ cylcoalkyl.

In other embodiments, R₆ and R₇ are taken together to form a substituted or unsubstituted heterocyclyl group. When R₆ and R₇ are taken together to form a substituted or unsubstituted heterocyclyl group, the heterocyclyl group is appended to the core through the nitrogen atom.

In other embodiments, said heterocyclyl group is selected from piperidinyl, pyrrolidinyl, azetidinyl, or aziridinyl. In other embodiments said heterocyclyl group (formed by R₆ and R₇) is morpholinyl, thiomorpholinyl, piperazinyl, N-methylpiperazinyl, or polyclic heterocycle such as quinuclidine.

In other embodiments R₆ and R₇ are taken together to form a substituted or unsubstituted heteroaryl group, such as a pyrrole, pyrazole, triazole, or pyridone group.

In other embodiments, R₁ is —N(CH₃)CH₂CH₂CH₃.

In still other embodiments, the compound is selected from:
1-(4-Amino-2-propylsulfanyl-imidazo[4,5-c]quinolin-1-yl)-2-methyl-propan-2-ol;
1-(4-Amino-2-azetidin-1-yl-imidazo[4,5-c]quinolin-1-yl)-2-methyl-propan-2-ol;
1-(4-Amino-2-pyrrolidin-1-yl-imidazo[4,5-c]quinolin-1-yl)-2-methyl-propan-2-ol;
1-(4-Amino-2-cyclopropylsulfanyl-imidazo[4,5-c]quinolin-1-yl)-2-methyl-propan-2-ol; or
1-(4-Amino-2-isobutylsulfanyl-imidazo[4,5-c]quinolin-1-yl)-2-methyl-propan-2-ol.

In still other embodiments, the compound of formula I is selected from the group consisting of:
N2-methyl-1-(2-methylpropyl)-1H-imidazo[4,5-c]quinoline-2,4-diamine;
N2,N2-dimethyl-1-(2-methylpropyl)-1H-imidazo[4,5-c]quinoline-2,4-diamine;
N2-ethyl-N2-methyl-1-(2-methylpropyl)-1H-imidazo[4,5-c]quinoline-2,4-diamine;
N2-methyl-1-(2-methylpropyl)-N2-propyl-1H-imidazo[4,5-c]quinoline-2,4-diamine;
1-(2-methylpropyl)-N2-propyl-1H-imidazo[4,5-c]quinoline-2,4-diamine;
N2-butyl-1-(2-methylpropyl)-1H-imidazo[4,5-c]quinoline-2,4-diamine;
N2-butyl-N2-methyl-1-(2-methylpropyl)-1H-imidazo[4,5-c]quinoline-2,4-diamine;
N2-methyl-1-(2-methylpropyl)-N2-pentyl-1H-imidazo[4,5-c]quinoline-2,4-diamine;
N2-methyl-1-(2-methylpropyl)-N2-prop-2-enyl-1H-imidazo[4,5-c]quinoline-2,4-diamine;
1-(2-methylpropyl)-2-[(phenylmethyl)thio]-1H-imidazo[4,5-c]quinolin-4-amine;
1-(2-methylpropyl)-2-(propylthio)-1H-imidazo[4,5-c]quinolin-4-amine;
2-[[4-amino-1-(2-methylpropyl)-1H-imidazo[4,5-c]quinolin-2-yl](methyl)amino]ethanol;
2-[[4-amino-1-(2-methylpropyl)-1H-imidazo[4,5-c]quinolin-2-yl](methyl)amino]ethyl acetate;
4-amino-1-(2-methylpropyl)-1,3-dihydro-2H-imidazo[4,5-c]quinolin-2-one;
N2-butyl-1-(2-methylpropyl)-N4,N4-bis(phenylmethyl)-1H-imidazo[4,5-c]quinoline-2,4-diamine;
N2-butyl-N2-methyl-1-(2-methylpropyl)-N4,N4-bis(phenylmethyl)-1H-imidazo[4,5-c]quinoline-2,4-diamine;
N2-methyl-1-(2-methylpropyl)-N4,N4-bis(phenylmethyl)-1H-imidazo[4,5-c]quinoline-2,4-diamine;
N2,N2-dimethyl-1-(2-methylpropyl)-N4,N4-bis(phenylmethyl)-1H-imidazo[4,5-c]quinoline-2,4-diamine;
1-{4-amino-2-[methyl(propyl)amino]-1H-imidazo[4,5-c]quinolin-1-yl}-2-methylpropan-2-ol;
1-[4-amino-2-(propylamino)-1H-imidazo[4,5-c]quinolin-1-yl]-2-methylpropan-2-ol; and
N4,N4-dibenzyl-1-(2-methoxy-2-methylpropyl)-N2-propyl-1H-imidazo[4,5-c]quinoline-2,4-diamine.

In some embodiments, the compound is selected from one of the following compounds:

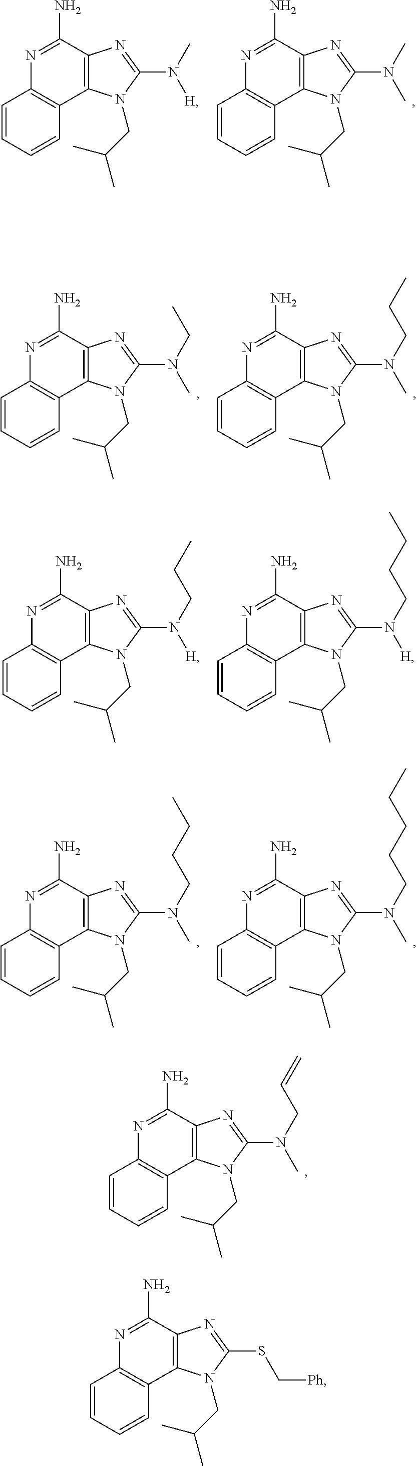

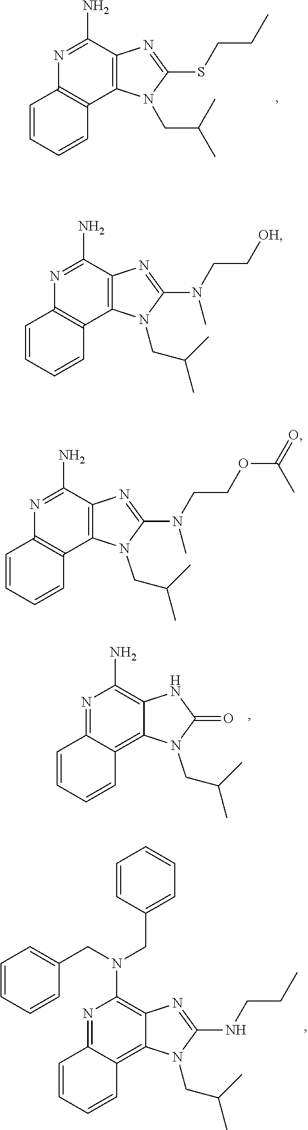

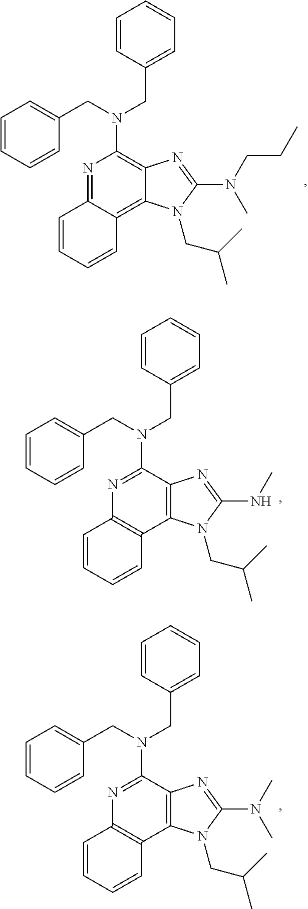 [SMILES: CCCN(C)c1nc2c(N(Cc3ccccc3)Cc3ccccc3)nc3ccccc3c2n1CC(C)C ; "," ; CNc1nc2c(N(Cc3ccccc3)Cc3ccccc3)nc3ccccc3c2n1CC(C)C ; "," ; CC(C)Cn1c(N(C)C)nc2c(N(Cc3ccccc3)Cc3ccccc3)nc3ccccc3c21 ; ","]

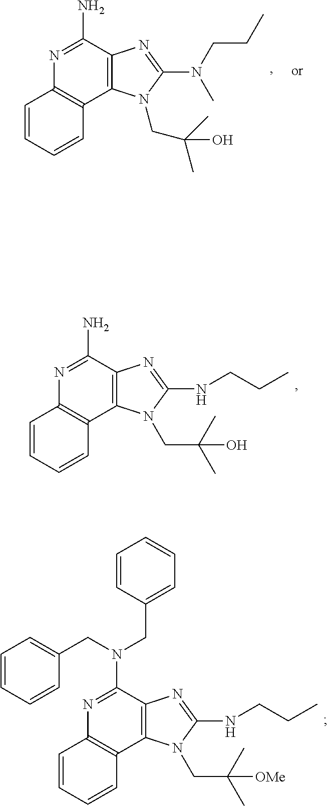

or pharmaceutically acceptable salts thereof, tautomers thereof or pharmaceutically acceptable salts of the tautomers.

In some other embodiments, the compound is selected from one of the following compounds:

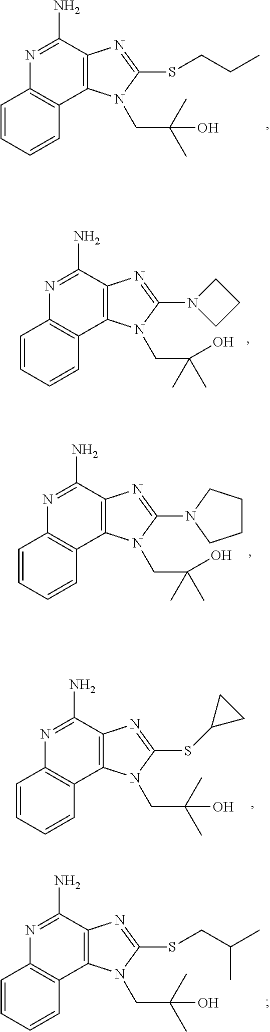

or pharmaceutically acceptable salts thereof, tautomers thereof or pharmaceutically acceptable salts of the tautomers.

In another embodiment, the invention provides a method of synthesizing a compound of formula (II)

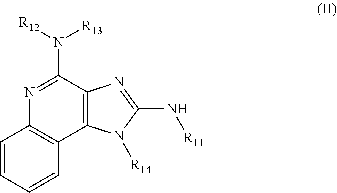

where R₁₁ and R₁₄ are each C₁₋₆ alkyl or substituted C₁₋₆ alkyl, and R₁₂ and R₁₃ are each a protecting group, comprising:
(a) reacting a compound of formula (III):

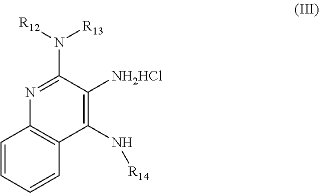

with an isothiocyanate of formula R₁₁NCS, where R₁₁ is defined above, thereby obtaining a compound of formula (IV):

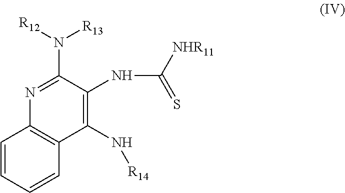

(b) optionally purifying the compound of formula (IV);
(c) reacting the compound of formula (IV) with a coupling agent, thereby obtaining a compound of formula (II); and
(d) optionally deprotecting the compound of formula (II).

In some embodiments of the method of synthesizing a compound of formula (II), the coupling agent is 1-(3-dimethylaminopropyl)-3-ethylcarbodiimide hydrochloride.

In other embodiments of the method of synthesizing a compound of any one of formulas (II-XIV), R₁₂ is a protecting group, such as tert-butoxycarbonyl (BOC), and R₁₃ is —H.

In another embodiment, the invention provides a method of synthesizing a compound of formula (V)

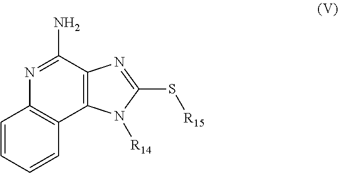

where R₁₄ is C₁₋₆ alkyl or substituted C₁₋₆ alkyl, and R₁₅ is C₆₋₁₀ aryl-C₁₋₆ alkyl, comprising:
(a) reacting a compound of formula (III):

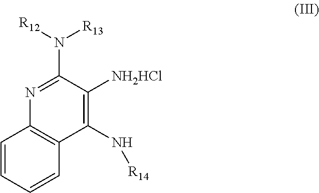

where R₁₂ and R₁₃ are each a protecting group, with carbon disulfide, thereby obtaining a compound of formula (VI):

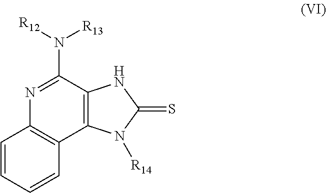

(b) optionally purifying the compound of formula (VI);
(c) reacting the compound of formula (VI) with an activated R₁₅ group to obtain a compound of formula (VIa);

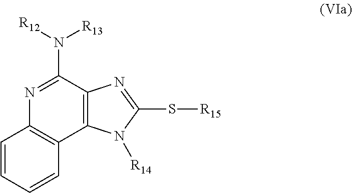

(d) deprotecting the compound of formula (VIa) thereby obtaining a compound of formula (V).

In another embodiment, the invention provides a method of synthesizing a compound of formula (VII)

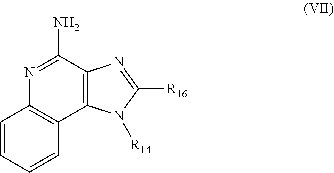

wherein R₁₄ is C₁₋₆ alkyl or substituted C₁₋₆ alkyl, and R₁₆ is —C(O)C₁₋₆ alkyl, or —C(O)O—C₁₋₆ alkyl, comprising:
(a) reacting a compound of formula (VIII):

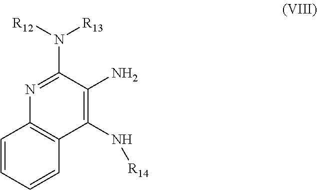

where R₁₂ and R₁₃ are each a protecting group, with a compound of formula (IX):

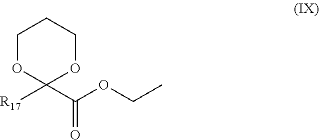

where R₁₇ is H or C₁₋₆ alkyl, thereby obtaining a compound of formula (X):

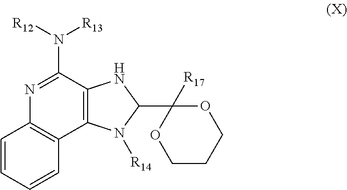

(b) optionally purifying the compound of formula (X); and
(c) reacting the compound of formula (X) with Pearlman's catalyst when R₁₇ is C₁₋₆ alkyl, and subsequently hydrolyzing the resulting compound under acidic conditions to give the compound of formula (VII); or
(d) hydrolyzing and then oxidizing the compound of formula (X) when R₁₇ is H, and subsequently reacting the resulting hydrolyzed and oxidized compound with a reagent to give the compound of formula (VIIa):

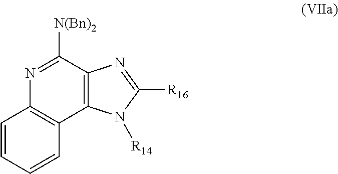

wherein Bn is benzyl, and further wherein the compound of formula (VIIa) is then reacted with hydrogen bromide to give the compound of formula (VII).

In another embodiment, the invention provides a method of synthesizing a compound of formula (XI)

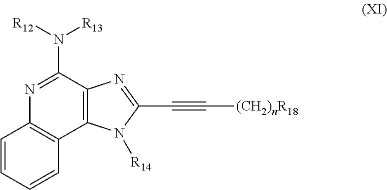

where R₁₂ and R₁₃ are each a protecting group, R₁₄ is C₁₋₆ alkyl or substituted C₁₋₆ alkyl, n is selected from 0, 1, 2, or 3, and R₁₈ is H, C₁₋₆ alkyl or C₆₋₁₀ aryl, comprising:
(a) reacting a compound of formula (III):

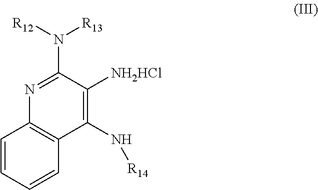

with a chloroformate of formula ClC(O)O—C₁₋₆ alkyl, thereby obtaining a compound of formula (XII):

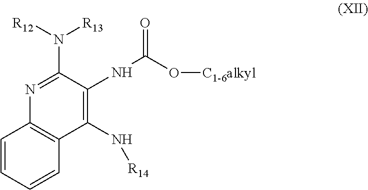

(b) optionally purifying the compound of formula (XII);
(c) reacting the compound of formula (XII) in the presence of an alkoxide base, thereby obtaining a compound of formula (XIII);

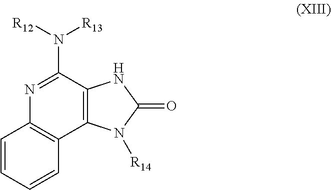

(d) reacting the compound of formula (XIII) with trifluoromethane sulfonic acid anhydride thereby obtaining a triflate of formula (XIV):

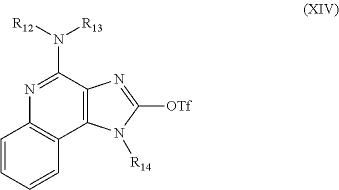

(e) reacting the compound of formula (XIV) with a lithium acetylide of formula Li—C≡C(CH₂)ₙR₁₈, wherein n and R₁₈ are as described above, thereby obtaining a compound of formula (XI); and
(f) optionally deprotecting the compound of formula (XI).

In some embodiments of each of the synthetic methods described herein, the protecting group R₁₂, or R₁₃, or both R₁₂ and R₁₃, is a benzyl group.

In another embodiment, the invention provides a method of synthesizing a compound of formula (XIV)

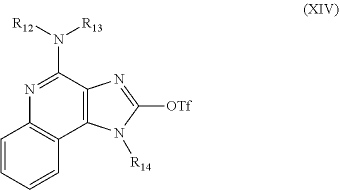

where R₁₂ and R₁₃ are each a protecting group or H, and R₁₄ is C₁₋₆ alkyl or substituted C₁₋₆ alkyl, comprising:
(a) reacting a compound of formula (III):

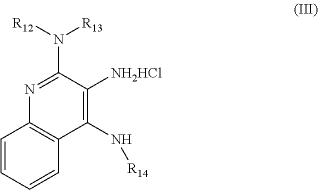

with a chloroformate of formula ClC(O)O—C₁₋₆ alkyl, thereby obtaining a compound of formula (XII):

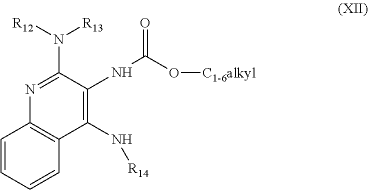

(b) optionally purifying the compound of formula (XII);
(c) reacting the compound of formula (XII) in the presence of an alkoxide base, thereby obtaining a compound of formula (XIII);

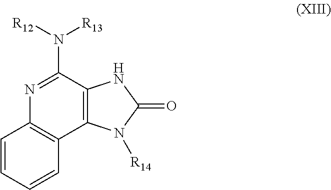

(d) reacting the compound of formula (XIII) with trifluoromethane sulfonic acid anhydride thereby obtaining a triflate of formula (XIV):

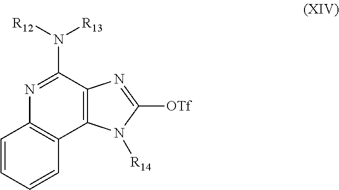

(e) optionally deprotecting the compound of formula (XIV).

In some embodiments, the compound of formula I is oxidized at the quinoline N atom such that the compound is an N-oxide, but otherwise has any of the other characteristics of the compound of formula I.

Further provided are compounds of formula I and mixtures thereof where any asymmetric carbon atom(s) can have either the R or S configuration. Substituents at a double bond or a ring of the compounds of formula I may be present in either the cis (-Z-) or trans (-E-) configurations. The compounds may thus be present as mixtures of isomers, diastereomers, and enantiomers or may be present as pure isomers. In some embodiments, the compounds are enantiomerically pure where only one enantiomer is present. In other embodiments, the compound may be present as a mixture of enantiomers which includes more of one enantiomer than it does of the other.

Generally, a SMIP or a composition comprising a SMIP is considered effective to elicit an immune response at a concentration of 300 μM or less in some embodiments, 200 M or less in some embodiments, 100 μM or less in some embodiments, or 20 vtM or less in some embodiments if the SMIP compound effects (a) the production of TNF-α in an in vitro cell based assay of human peripheral blood mononuclear cells, and (b) a concentration of human peripheral blood mononuclear cells (PBMCs) of about 500,000/mL, when the cells are exposed to the compound for a period of about 18-24 hours, preferably about 24 hours.

The above method of stimulating a local immune response, for example in selected cells or tissues of a patient, includes the stimulation of a local immune response where the selected cells or tissues are infected or cancerous. In some embodiments, the selected cells or tissues are infected with a fungus or bacterium. In some embodiments, the selected tissues are inflamed with an allergen, for example in an asthmatic condition. In other embodiments, the selected cells are infected with a virus or bacteria. In still other embodiments, the infectious agent is HCV, HIV, HBV, HSV, H. pylori, HSV Type 1 or 2, or Human Papilloma Virus.

Another embodiment provides a method of inducing interferon biosynthesis in a subject. Such methods include administering a compound of formula I to the subject in an amount sufficient to induce interferon biosynthesis. In some such methods, a vaccine adjuvant of formula I is administered to the subject in an amount sufficient to induce interferon biosynthesis.

Another embodiment provides a compound of formula I, wherein the compound is co-administered with another agent to a patient in need thereof. In some such embodiments, the agent is an antigen or a vaccine. In embodiments, where the compound of formula I is co-administered to a patient or subject along with another agent, the compound of formula I may be administered to the subject before, during, or after the other agent is administered to the subject. Therefore, in some embodiments, the compound of formula I is administered to the subject at the same time that the other agent is administered to the subject.

Another embodiment provides a method of modulating an immune response in a subject. Such methods include administering a compound of formula I to the subject.

Another embodiment provides a method for inducing the production of TNF-α in a subject. Such methods include administering a compound of formula I to a subject in an amount sufficient to induce the production of TNF-α. In some such embodiment thereof, the compound has an average steady state drug concentration in the blood of less than 20 μM.

Another embodiment provides a method of inducing an immune response in a subject. The embodiment includes administering a compound of formula I to the subject in an amount sufficient to induce an immune response. In some such embodiments, the immune response involves the production of cytokines or increased production of TNF-α.

Another embodiment provides a method of inducing an immune response in a subject suffering from a microbial infection. The method includes administering a compound of formula I to the subject in an amount sufficient to induce an immune response.

Another embodiment provides a method of inducing an immune response in a subject suffering from a viral infection or a disease condition caused by a virus. The method includes administering a compound of formula I to the subject in an amount sufficient to induce an immune response in the subject. In some such embodiments, the subject is suffering from a viral infection or disease condition caused by the hepatitis C virus (HCV). In other embodiments, the subject is suffering from a viral infection or disease condition caused by the human immunodeficiency virus (HIV). In another embodiment or method, the compound of formula I is administered topically to a subject.

Another embodiment provides a method of inducing an immune response in a subject for prevention of a viral infection or a disease condition caused by a virus. The method includes administering a compound of formula I to the subject in an amount sufficient to induce an immune response in the subject. In some such embodiments, the subject is prevented from a viral infection or disease condition. In other embodiments, the subject is protected from a microbial or other pathogenic infection, such as a those described herein.

Another embodiment provides a method of inducing an immune response in a subject suffering from an abnormal cellular proliferation or cancer. The method includes administering a compound of formula I to the subject in an amount sufficient to induce an immune response. In some embodiments, the compound is administered to a subject that is suffering from a disease associated with abnormal cellular proliferation. In some such embodiments, the disease is selected from neuro-fibromatosis, atherosclerosis, pulmonary fibrosis, arthritis, psoriasis, glomerulonephritis, restenosis, proliferative diabetic retinopathy (PDR), hypertrophic scar formation, inflammatory bowel disease, transplantation rejection, angiogenesis, or endotoxic shock.

Other embodiments provide methods of inducing an immune response in a subject suffering from an allergic disease. Such methods include administering a compound of formula I to the subject in an amount sufficient to induce an immune response.

Another embodiment provides a method of inducing an immune response in a subject suffering from asthma. The method includes administering a compound of formula I to the subject in an amount sufficient to induce an immune response. In some embodiments, asthma may be treated by steering the immune response away from Type 2 cytokine secretion and effector mechanism (e.g., IgE production and/or mast cell/basophil activation).

Another embodiment provides a method of inducing an immune response in a subject suffering from precancerous lesions. The method includes administering a compound of formula I to the subject in an amount sufficient to induce an immune response. In some such embodiments, the precancerous lesions are actinic keratosis. In other embodiments, the precancerous lesions are selected from actinic keratosis, atypical or dysplastic nevi, or premalignant lentigos. In another embodiment or method, the compound of formula I is administered topically to a subject.

Other embodiments provide a method of inhibiting a kinase in a subject. Such methods include administering the compound of formula I to the subject.

Another embodiment provides a method of modulating an immune response in a subject. The method includes administering a compound of formula I to the subject in an amount sufficient to inhibit a kinase in the subject. In some such embodiments, the kinase is selected from EGFr, c-Kit, bFGF, Kdr, CHK1, CDK, cdc-2, Akt, PDGF, PI3K, VEGF, PKA, PKB, src, c-Met, Abl, Ras, RAF, MEK, or combinations thereof. In another embodiment or method, the compound of formula I is administered topically to a subject.

Another embodiment provides a method of inducing an immune response in a subject, comprising: administering to the subject a compound of formula I and an antigen, wherein the compound induces or enhances an immune response to the antigen in the subject. More particularly the antigen is influenza or any other antigen described herein.

Another embodiment provides a composition comprising: the compound of formula I and another agent. In some embodiments, the other agent is an immunogenic composition. In further embodiments, the agent is an antigen. In still further embodiments, the agent is a vaccine and the compound is a vaccine adjuvant. In another embodiment, the composition further comprises poly(lactide-co-glycolide) (PLG). In another embodiment, the composition further comprises MF59 or another adjuvant.

In another embodiment or method, the compound of formula I is administered topically to a subject.

Another embodiment provides a pharmaceutical composition, comprising: the compound of formula I and a pharmaceutically acceptable excipient.

In another embodiment, the compound of formula I is administered topically. More particularly the compound is administered topically to a lesion caused by a viral infection. More particularly the viral infection is Herpes simplex virus (HSV), more particular still, Type II Herpes simplex virus. In another embodiment the virus is human Papilloma virus (HPV). Alternatively, the compound of formula I is administered topically to a lesion caused by actinic keratosis.

Another embodiment of the present invention provides a method of stimulating TLR-7 production comprising administering a compound of formula I. Another embodiment provides a method of stimulating TLR-8 production comprising administering a compound of formula I. Another embodiment provides a method of stimulating TLR-7 and TLR-8 production comprising administering a compound of formula I.

Compounds of the present invention cause immune potentiation and stimulate production of TLR-7 and TLR-8. Such compounds can be used as polyclonal activators for the production of antigens. More particularly the invention relates to a method of preparing monoclonal antibodies with a desired antigen specificity comprising contacting the compounds of the present invention (such as those of formula I) with immortalized memory B cells.

The monoclonal antibodies produced therefrom, or fragments thereof may be used for the treatment of disease, for the prevention of disease or for the diagnosis of disease. Methods of diagnosis may include contacting an antibody or an antibody fragment with a sample. The methods of diagnosis may also include the detection of an antigen/antibody complex.

The memory B cells to be transformed can come from various sources (e.g. from whole blood, from peripheral blood mononuclear cells (PBMCs), from blood culture, from bone marrow, from organs, etc.), and suitable methods for obtaining human B cells are well known in the art. Samples may include cells that are not memory B cells or other blood cells. A specific human memory B lymphocyte subpopulation exhibiting a desired antigen specificity may be selected before the transformation step by using methods known in the art. In one embodiment, the human memory B lymphocyte subpopulation has specificity for a virus e.g. the B cells are taken from a patient who is suffering or has recovered from the virus. In another embodiment, B cells are taken from subjects with Alzheimer's disease and include B cells with specificity for B-amyloid (e.g. Mattson & Chan (2003) Science 301:1 847-9; etc.).

Another embodiment provides a method for producing immortalized B memory lymphocytes, comprising the step of transforming B memory lymphocytes using the Epstein Barr virus in the presence of a compound of the present invention, such as a compound of Formula I. See WO 04/76677.

The invention also provides pharmaceutical compositions that include any of the aforementioned compounds or embodiments of formula I. Such compositions may include other pharmaceutically acceptable ingredients such as one or more of excipients, carriers, and the like well-known to those skilled in the art.

It is contemplated that the invention encompasses all possible combinations of the preceding embodiments. In some embodiments of each of the compound and methods described herein, R₄ and R₅ of the compounds of formula (I) are each H.

The imidazoquinoline compounds can be used with or without an antigen in therapeutic applications, for example to treat cancer or infectious diseases. The imidazoquinoline compounds may also be used in combination with other therapeutic agents, such as anti-viral agents and monoclonal antibodies in different therapeutic applications.

One embodiment of the method of inducing an immunostimulatory effect in a patient is directed to administering an immunogenic composition comprising a vaccine in an amount effective to stimulate an immune response such as a cell-mediated immune response and, as a vaccine adjuvant, an imidazoquinoline compound, in an amount effective to potentiate the immune response such as the cell-mediated immune response to the vaccine.

Agents combined with the imidazoquinoline compounds, contemplated to be useful in treating the aforementioned diseases include those well known in the art, such as, but not limited to, anesthetics, hypnotic sedatives, anti-anxieties, antiepileptics, antipyretic antiphlogistics, stimulants, wake amines, anti-parkinson drugs, agents for psychoneuroses, agents for central nervous system, skeletal muscle relaxants, agents for autonomic nervous system, antispastic agents, cytotoxic agents, monoclonal antibodies, drugs for eye, drugs for nose and ear, anti-vertiginous drugs, cardiotonics, antiarrhythmic drugs, diuretics, pressure reduction drugs, vasoconstrictors, coronary vaso-dilators, peripheral vasodilating drugs, hyper-lipemia drugs, breath stimulants, antitussive and expectorant drugs, bronchodilators, drugs for allergy, antidiarrheal drugs, drugs for intestinal disorders, peptic ulcer drugs, stomachic digestants, antacids, cholagogouses, pituitary hormone drugs, salivary gland hormones, thyroid hormone drugs, antithyroid drugs, anabolic steroids, corticosteroids, androgen drugs, estrogen drugs, corpus luteum hormone drugs, mixed hormones, urinary/genital organ drugs, anus drugs, surgical sterilizations/antiseptics, wound protectives, externals for purulent diseases, analgesics, antipruritics, astringents, antiphlogistics, externals for parasite skin diseases, skin-softening drugs, caustics, dental/oral drugs, vitamins, inorganic preparations, supplemental liquids, hemostatics, anticoagulation drugs, drugs for liver diseases, antidotes, habitual intoxication drugs, drugs for treatment of gout, enzyme preparations, diabetic drugs, antioncotics, antihistaminics, antibiotics (such as ketolides, aminoglycosides, sulphonamides, and/or beta lactams), chemotherapeutics, biological preparations, anthelmintics, anti-Protozoas, drugs for preparations, X-ray contrast media, and diagnostic drugs.

Further methods of the invention are provided wherein compositions described herein are used for the treatment of cancer and reduction of tumor growth. In one aspect, an imidazoquinoline compound of the invention is combined with a known mAb for the treatment of cancer. In one such embodiment, an antibody and an imidazoquinoline compound are administered to a subject in need thereof. In some such embodiments, the antibody, individually, has an inhibiting effect upon tumor cell growth, and the imidazoquinoline compound induces the production of cytokines.

In accordance with another embodiment of the present invention, a therapeutic composition for inhibiting tumor cell growth in a subject is provided. Such compositions include an effective amount of a combination of at least one imidazoquinoline compound, at least one mAb, and at least one pharmaceutically acceptable carrier. In such embodiments, the combination is more effective at inhibiting the growth of certain mammalian tumor cells than are any of the agents when individually administered.

In another embodiment, methods of treating cancer are provided in which known anticancer agents are combined with imidazoquinoline compounds to reduce tumor growth in a subject. A number of suitable anticancer agents are contemplated for use in such methods. Indeed, the present invention contemplates, but is not limited to, administration of numerous anticancer agents including, but not limited to: fenretinide, vatalanib, SU-11248, SU 5416, SU 6668, oxaliplatin, bortezomib, R 115777, CEP-701, ZD-6474, MLN-518, lapatinib, gefitinib (iressa), erlotinib (tarceva), perifosine, CYC-202, LY-317615, squalamine, UCN-01, midostaurin, irofulven, staurosporine, alvocidib, genistein, DA-9601, avicine, docetaxel, IM 862, SU 101, and tetrathiomolybdate as well as other agents that induce apoptosis, such as, but not limited to polynucleotides (e.g., ribozymes); polypeptides (e.g., enzymes); drugs; biological mimetics; 25 alkaloids; alkylating agents; antitumor antibiotics; antimetabolites; hormones; platinum compounds; monoclonal antibodies conjugated with anticancer drugs, toxins, and/or radionuclides; biological response modifiers (e.g., interferons [e.g., IFN-a, etc.] and interleukins [e.g., IL-2, etc.], etc.); adoptive immunotherapy agents; hematopoietic growth factors; agents that induce tumor cell differentiation (e.g., all-trans-retinoic acid, etc.); gene 30 therapy reagents; antisense therapy reagents and nucleotides; tumor vaccines; and inhibitors of angiogenesis, and the like. Numerous other examples of chemotherapeutic compounds and anticancer therapies suitable for co-administration with the disclosed imidazoquinoline compounds will be known and apparent to those skilled in the art.

In some embodiments, anticancer agents comprise agents that induce or stimulate apoptosis. Agents that induce apoptosis include, but are not limited to, radiation (e.g., W); kinase inhibitors (e.g., Epidermal Growth Factor Receptor [EGFR] kinase; inhibitor, Vascular Growth Factor Receptor [VGFR] kinase inhibitor, Fibroblast Growth 5 Factor Receptor [FGFR] kinase inhibitor, Platelet-derived Growth Factor Receptor [PGFR]I kinase inhibitor, EGFr and Bcr-Abl kinase inhibitors such as Gleevec, Iressa, and Tarceva]); antisense molecules; antibodies [e.g., Herceptin and Rituxan]; anti-estrogens [e.g., raloxifene and tamoxifen]; anti-androgens [e.g., flutamide, bicalutamide, finasteride, aminoglutethamide, ketoconazole, and corticosteroids]; cyclooxygenase 2 (COX-2) inhibitors [e.g., Celecoxib, meloxicam, NS-398, and non-steroidal

Anti-inflammatory drugs I (NSAIDs)]; and cancer chemotherapeutic drugs [e.g., CPT-11, fludarabine (Fludara), dacarbazine (DTIC), dexamethasone, mitoxantrone, Mylotarg, cisplatinum, 5-FU, Doxrubicin, Taxotere or taxol]; cellular signaling molecules; ceramides and cytokines; and the like may also be administered to subjects in conjunction with the imidazoquinolines of formula I.

In other embodiments, methods of treating allergies are provided. Such methods include administering an imidazoquinoline compound alone or in combination with another agent known to be effective against allergies. In such embodiments, the combination is more effective in treating an allergic condition than the known agent(s) is/are without the addition of the imidazoquinoline compound. In some such embodiments, the known agent is an antihistamine and/or a leukotriene inhibitor. In other embodiments, the allergic condition is asthma. In other embodiments, the allergic condition is selected from allergic rhinitis, dermatosis, or urticaria. In some such embodiments, the combination is administered to the subject enterally, parenterally, intranasally, subcutaneously, or intraarterially.

Vaccine compositions contemplated to be within the scope of the present invention may include (an) additional adjuvant(s). In some embodiments, adjuvants to enhance effectiveness of the composition include, but are not limited to: (1) aluminum salts (alum), such as aluminum hydroxide, aluminum phosphate, aluminum sulfate, etc; (2) oil-in-water emulsion formulations (with or without specific immunostimulating agents such as muramyl peptides or bacterial cell wall components), such as, for example (a) MF59™ (WO 90/14837), containing 5% squalene, 0.5% Tween 80, and 0.5% Span 85 (optionally containing MTP-PE) formulated into submicron particles using a microfluidizer, (b) SAF, containing 5% squalene, 0.5% Tween 80, 5% pluronic-blocked polymer L121, and thr-MDP either microfluidized into a submicron emulsion or vortexed to generate a larger particle size emulsion, and (c) Ribi™ adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); (3) saponin adjuvants, such as QS21 or Stimulon™ (Cambridge Bioscience, Worcester, Mass.) may be used or particles generated therefrom such as ISCOMs (immunostimulating complexes), which ISCOMs may be devoid of additional detergent e.g. WO 00/07621; (4) Complete Freund's Adjuvant (CFA) and Incomplete Freund's Adjuvant (IFA); (5) cytokines, such as interleukins (e.g. IL-1, IL-2, IL-4, IL-5, IL-6, IL-7, IL-12 (WO 99/44636), etc.), interferons (e.g. gamma interferon), macrophage colony stimulating factor (M-CSF), tumor necrosis factor (TNF), etc.; (6) momophosphoryl lipid A (MPL) or 3-O-deacylated MPL (3dMPL), optionally in the substantial absence of alum when used with pneumococcal saccharides e.g. WO 00/56358; and RC529 (7) combinations of 3dMPL with, for example, QS21 and/or oil-in-water emulsions e.g. EP-A-0835318; (8) oligonucleotides comprising CpG motifs, i.e. containing at least one CG dinucleotide, with 5-methylcytosine optionally being used in place of cytosine; (9) a polyoxyethylene ether or a polyoxyethylene ester e.g. WO 99/52549; (10) a polyoxyethylene sorbitan ester surfactant in combination with an octoxynol (WO 0121207) or a polyoxyethylene alkyl ether or ester surfactant in combination with at least one additional non-ionic surfactant such as an octoxynol (WO 01/21152); (11) a saponin and an immunostimulatory oligonucleotide (e.g. a CpG oligonucleotide) (WO 00/62800); (12) an immunostimulant and a particle of metal salt e.g., WO 00/23105; (13) a saponin and an oil-in-water emulsion e.g. WO 99/11241; (14) a saponin (e.g. QS21)+3dMPL+IL-12 (optionally+a sterol) e.g. WO 98/57659; (14) other substances that act as immunostimulating agents to enhance the effectiveness of the composition. In some embodiments, Alum (especially aluminum phosphate and/or hydroxide) and MF59 are used with saccharide antigens.

The invention is also directed to methods of administering vaccine compositions. In some embodiments, the vaccine is administered to the subject in an amount effective to stimulate an immune response. The amount that constitutes an effective amount depends, inter alia, on the particular vaccine used, the particular adjuvant compound being administered and the amount thereof, the immune response that is to be enhanced (humoral or cell mediated), the state of the immune system (e.g., suppressed, compromised, stimulated), and the desired therapeutic result. Accordingly it is not practical to set forth generally the amount that constitutes an effective amount of the vaccine. Those of ordinary skill in the art, however, can readily determine the appropriate amount with due consideration of such factors.

The vaccine compositions of the invention can be administered to various animals subjects including mammals such as human and non-human subjects, including, for example, pocket pets, fowl, and the like according to conventional methods well-known to those skilled in the art (e.g., orally, subcutaneously, nasally, topically).

Suitable vaccines include, but are not limited to, any material that raises either or both humoral or cell mediated immune response. Suitable vaccines include live viral and bacterial antigens and inactivated viral, tumor-derived, protozoal, organism-derived, fungal, and bacterial antigens, toxoids, toxins, polysaccharides, proteins, glycoproteins, peptides, and the like. Conventional vaccines, such as those used in connection with BCG (live bacteria), cholera, plague, and typhoid (killed bacteria), hepatitis B, influenza, inactivated polio, and rabies (inactivated virus), measles, mumps, rubella, oral polio, SARS vaccines, and yellow fever (live virus), tetanus and diphtheria (toxoids), hemophilus influenzae b, meningococcal, and pneumococcal (bacterial polysaccharides) can also be used. Any antigen known in the art or disclosed herein may be used in accordance with the invention.

Furthermore, it is contemplated that certain currently experimental vaccines, especially materials such as recombinant proteins, glycoproteins, and peptides that do not raise a strong immune response, will also find use in connection with the imidazoquinoline compounds of the invention. Exemplary experimental subunit antigens include, but are not limited to, those related to viral disease such as adenovirus, acquired immune deficiency syndrome (AIDS), chicken pox, cytomegalovirus, dengue, feline leukemia, fowl plague, hepatitis A, hepatitis B, hepatitis C, HSV-1, HSV-2, hog cholera, influenza A, influenza B, Japanese encephalitis, measles, parainfluenza, rabies, respiratory syncytial virus, SARS virus, rotavirus, wart, and yellow fever.

Specific antigens for use with the invention include, but are not limited to, those listed below. The number(s) in parenthesis indicate representative resources of the antigen. The resource list follows the antigen list and each resource is hereby incorporated by reference in its entirety and for all purposes as if fully set forth herein.

Specific antigens include: a protein antigen from N. meningitides serogroup B (1-7); an outer-membrane vesicle (OMV) preparation from N. meningitides serogroup B. (8, 9, 10, 11); a saccharide antigen from N. meningitides serogroup A, C W135 and/or Y, such as the oligosaccharide (12) from serogroup C (13); a saccharide antigen from Streptocaccus pneumoniae (14, 15, 16); an antigen from N. gonorrhoeae (1, 2, 3); an antigen from Chlamydia pneumoniae (17, 18, 19, 20, 21, 22, 23); an antigen from Chlamydia trachomnatis (24); an antigen from hepatitis A virus, such as inactivated virus (25, 26); an antigen from hepatitis B virus, such as the surface and/or core antigens (e.g. 26, 27); an antigen from hepatitis C virus (28); an antigen from Bordetella pertussis, such as pertussis holotoxin (PT) and filamentous haemagglutinin (FHA) from B. pertussis, optionally also combination with pertactin and/or agglutinogens 2 and 3 (29, 30); a diphtheria antigen, such as a diphtheria toxoid (31:chapter 3) e.g. the CRM₁₉₇ mutant (32); a tetanus antigen, such as a tetanus toxoid (31:chapter 4); a protein antigen from Helicobacter pylori such as CagA (33), VacA (33), NAP (34), HopX (5), HopY (35) and/or urease; a saccharide antigen from Haemophilus influenzae B (13); an antigen from Porphyromnonas gingivalis (36); polio antigen(s) (37, 38) such as IPV or OPV; rabies antigen(s) (39) such lyophilized inactivated virus (40, RabAvert™); measles, mumps and/or rubella antigens (31: chapters 9, 10, & 11); influenza antigen(s) (31:chapter 19), such as the haemagglutinin and/or neuraminidase surface proteins; an antigen from Moraxella catarrhalis (41); an antigen from Streptococcus agalactiae (group B streptococcus) (42, 43); an antigen from Streptococcus pyogenes (group A streptococcus) (43, 44, 45); and an antigen from Staphylococcus aureus (46). The compositions of the invention may include one or more of the above antigens.

In some embodiments, the small molecule immune potentiator compounds of the invention are used in adjuvant systems, in compositions for administering influenza vaccines. In some such embodiments, one or more small molecule immune potentiator compounds of the invention are employed, optionally along with another adjuvant such as MF59 adjuvant, and one or more influenza antigen(s) (31:chapter 19), such as the haemagglutinin and/or neuraminidase surface proteins.

In embodiments where a saccharide or carbohydrate antigen is used, is the saccharide or carbohydrate antigen may be conjugated to a carrier protein in order to enhance antigenicity (47-56). In some embodiments, carrier proteins are bacterial toxine or toxoids, such as diphtheria or tetanus toxoids. The CRM₁₉₇ diphtheria toxoid is an example of one such toxoid. Other suitable carrier proteins include the N. meningitides outer membrane protein (57), synthetic peptides (58, 59), heat shock proteins (60), pertussis proteins (61, 62), protein D from H. influenzae (63), toxin A or B from C. difficile (64) etc. In embodiments where a mixture comprises capsular saccharides from both serogroups A and C, the ratio (w/w) of MenA saccharide:MenC saccharide may be greater than 1 (e.g. 2:1, 3:1, 4:4, 5:1, 10:1 or higher). Saccharides from different serogroups of N. meningitides may be conjugated to the same or different carrier proteins.

Any suitable conjugation reaction can be used, with any suitable linker where necessary. Toxic protein antigens may be detoxified where necessary (e.g., detoxification of pertussis toxin by chemical and/or genetic means (30)). Where a diphtheria antigen is included in the composition, it is preferred to also include tetanus antigens and pertussis antigens. Similarly, where a tetanus antigen is included, it is preferred to also include diphtheria and pertussis antigens. Similarly, where pertussis antigen is included, it is preferred to also include diphtheria and tetanus antigens.

### Adjuvants:

Vaccines of the invention may be administered in conjunction with other immunoregulatory agents. In particular, compositions will usually include an adjuvant. Adjuvants for use with the invention include, but are not limited to, one or more of the following set forth below:

### A. Mineral Containing Compositions

Mineral containing compositions suitable for use as adjuvants in the invention include mineral salts, such as aluminum salts and calcium salts. The invention includes mineral salts such as hydroxides (e.g. oxyhydroxides), phosphates (e.g. hydroxyphosphates, orthophosphates), sulfates, etc. (e.g. see chapters 8 & 9 of Vaccine Design . . . (1995) eds. Powell & Newman. ISBN: 030644867X. Plenum.), or mixtures of different mineral compounds (e.g. a mixture of a phosphate and a hydroxide adjuvant, optionally with an excess of the phosphate), with the compounds taking any suitable form (e.g. gel, crystalline, amorphous, etc.), and with adsorption to the salt(s) being preferred. The mineral containing compositions may also be formulated as a particle of metal salt (WO00/23105).

Aluminum salts may be included in vaccines of the invention such that the dose of Al³⁺ is between 0.2 and 1.0 mg per dose.

In one embodiment the aluminum based adjuvant for use in the present invention is alum (aluminum potassium sulfate (AlK(SO₄)₂)), or an alum derivative, such as that formed in-situ by mixing an antigen in phosphate buffer with alum, followed by titration and precipitation with a base such as ammonium hydroxide or sodium hydroxide.

Another aluminum-based adjuvant for use in vaccine formulations of the present invention is aluminum hydroxide adjuvant (Al(OH)₃) or crystalline aluminum oxyhydroxide (AlOOH), which is an excellent adsorbant, having a surface area of approximately 500 m²/g. Alternatively, aluminum phosphate adjuvant (AlPO₄) or aluminum hydroxyphosphate, which contains phosphate groups in place of some or all of the hydroxyl groups of aluminum hydroxide adjuvant is provided. Preferred aluminum phosphate adjuvants provided herein are amorphous and soluble in acidic, basic and neutral media.

In another embodiment the adjuvant of the invention comprises both aluminum phosphate and aluminum hydroxide. In a more particular embodiment thereof, the adjuvant has a greater amount of aluminum phosphate than aluminum hydroxide, such as a ratio of 2:1, 3:1, 4:1, 5:1, 6:1, 7:1, 8:1, 9:1 or greater than 9:1, by weight aluminum phosphate to aluminum hydroxide. More particular still, aluminum salts in the vaccine are present at 0.4 to 1.0 mg per vaccine dose, or 0.4 to 0.8 mg per vaccine dose, or 0.5 to 0.7 mg per vaccine dose, or about 0.6 mg per vaccine dose.

Generally, the preferred aluminum-based adjuvant(s), or ratio of multiple aluminum-based adjuvants, such as aluminum phosphate to aluminum hydroxide is selected by optimization of electrostatic attraction between molecules such that the antigen carries an opposite charge as the adjuvant at the desired pH. For example, aluminum phosphate adjuvant (iep=4) adsorbs lysozyme, but not albumin at pH 7.4. Should albumin be the target, aluminum hydroxide adjuvant would be selected (iep 11.4). Alternatively, pretreatment of aluminum hydroxide with phosphate lowers its isoelectric point, making it a preferred adjuvant for more basic antigens.

### B. Oil-Emulsions

Oil-emulsion compositions suitable for use as adjuvants in the invention include squalene-water emulsions, such as MF59 (5% Squalene, 0.5% Tween 80, and 0.5% Span 85, formulated into submicron particles using a microfluidizer). See WO90/14837. See also, Podda, “The adjuvanted influenza vaccines with novel adjuvants: experience with the MF59-adjuvanted vaccine”, Vaccine (2001) 19: 2673-2680; Frey et al., “Comparison of the safety, tolerability, and immunogenicity of a MF59-adjuvanted influenza vaccine and a non-adjuvanted influenza vaccine in non-elderly adults”, Vaccine (2003) 21:4234-4237. MF59 is used as the adjuvant in the FLUAD™ influenza virus trivalent subunit vaccine.

Particularly preferred adjuvants for use in the compositions are submicron oil-in-water emulsions. Preferred submicron oil-in-water emulsions for use herein are squalene/water emulsions optionally containing varying amounts of MTP-PE, such as a submicron oil-in-water emulsion containing 4-5% w/v squalene, 0.25-1.0% w/v Tween 80™ (polyoxyelthylenesorbitan monooleate), and/or 0.25-1.0% Span 85™ (sorbitan trioleate), and, optionally, N-acetylmuramyl-L-alanyl-D-isogluatminyl-L-alanine-2-(1′-2′-dipalmitoyl-sn-glycero-3-huydroxyphosphosphoryloxy)-ethylamine (MTP-PE), for example, the submicron oil-in-water emulsion known as “MF59” (International Publication No. WO90/14837; U.S. Pat. Nos. 6,299,884 and 6,451,325, and Ott et al., “MF59—Design and Evaluation of a Safe and Potent Adjuvant for Human Vaccines” in Vaccine Design: The Subunit and Adjuvant Approach (Powell, M. F. and Newman, M. J. eds.) Plenum Press, New York, 1995, pp. 277-296). MF59 contains 4-5% w/v Squalene (e.g. 4.3%), 0.25-0.5% w/v Tween 80™, and 0.5% w/v Span 85™ and optionally contains various amounts of MTP-PE, formulated into submicron particles using a microfluidizer such as Model 110Y microfluidizer (Microfluidics, Newton, Mass.). For example, MTP-PE may be present in an amount of about 0-500.1 g/dose, more preferably 0-250 μg/dose and most preferably, 0-100 g/dose. As used herein, the term “MF59-0” refers to the above submicron oil-in-water emulsion lacking MTP-PE, while the term MF59-MTP denotes a formulation that contains MTP-PE. For instance, “MF59-100” contains 100 μg MTP-PE per dose, and so on. MF69, another submicron oil-in-water emulsion for use herein, contains 4.3% w/v squalene, 0.25% w/v Tween 80™, and 0.75% w/v Span 85™ and optionally MTP-PE. Yet another submicron oil-in-water emulsion is MF75, also known as SAF, containing 10% squalene, 0.4% Tween 80™, 5% pluronic-blocked polymer L121, and thr-MDP, also microfluidized into a submicron emulsion. MF75-MTP denotes an MF75 formulation that includes MTP, such as from 100-400 μg MTP-PE per dose.

Submicron oil-in-water emulsions, methods of making the same and immunostimulating agents, such as muramyl peptides, for use in the compositions, are described in detail in International Publication No. WO90/14837 and U.S. Pat. Nos. 6,299,884 and 6,451,325.

Complete Freund's adjuvant (CFA) and incomplete Freund's adjuvant (IFA) may also be used as adjuvants in the invention.

### C. Saponin Formulations

Saponin formulations, may also be used as adjuvants in the invention. Saponins are a heterologous group of sterol glycosides and triterpenoid glycosides that are found in the bark, leaves, stems, roots and even flowers of a wide range of plant species. Saponins isolated from the bark of the Quillaia saponaria Molina tree have been widely studied as adjuvants. Saponins can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs.

Saponin compositions have been purified using High Performance Thin Layer Chromatography (HP-TLC) and Reversed Phase High Performance Liquid Chromatography (RP-HPLC). Specific purified fractions using these techniques have been identified, including QS7, QS17, QS18, QS21, QH-A, QH-B and QH-C. Preferably, the saponin is QS21. A method of production of QS21 is disclosed in U.S. Pat. No. 5,057,540. Saponin formulations may also comprise a sterol, such as cholesterol (see WO96/33739).

Combinations of saponins and cholesterols can be used to form unique particles called Immunostimulating Complexes (ISCOMs). ISCOMs typically also include a phospholipid such as phosphatidylethanolamine or phosphatidylcholine. Any known saponin can be used in ISCOMs. Preferably, the ISCOM includes one or more of Quil A, QHA and QHC. ISCOMs are further described in EP0109942, WO96/11711 and WO96/33739. Optionally, the ISCOMS may be devoid of (an) additional detergent(s). See WO00/07621.

A review of the development of saponin based adjuvants can be found in Barr, et al., “ISCOMs and other saponin based adjuvants”, Advanced Drug Delivery Reviews (1998) 32:247-271. See also Sjolander, et al., “Uptake and adjuvant activity of orally delivered saponin and ISCOM vaccines”, Advanced Drug Delivery Reviews (1998) 32:321-338.

### D. Virosomes and Virus Like Particles (VLPs)

Virosomes and Virus Like Particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in WO03/024480, WO03/024481, and Niikura et al., “Chimeric Recombinant Hepatitis E Virus-Like Particles as an Oral Vaccine Vehicle Presenting Foreign Epitopes”, Virology (2002) 293:273-280; Lenz et al., “Papillomarivurs-Like Particles Induce Acute Activation of Dendritic Cells”, Journal of Immunology (2001) 5246-5355; Pinto, et al., “Cellular Immune Responses to Human Papillomavirus (HPV)-16 L1 Healthy Volunteers Immunized with Recombinant HPV-16 L1 Virus-Like Particles”, Journal of Infectious Diseases (2003) 188:327-338; and Gerber et al., “Human Papillomavrisu Virus-Like Particles Are Efficient Oral Immunogens when Coadministered with Escherichia coli Heat-Labile Entertoxin Mutant R192G or CpG”, Journal of Virology (2001) 75(10):4752-4760. Virosomes are discussed further in, for example, Gluck et al., “New Technology Platforms in the Development of Vaccines for the Future”, Vaccine (2002) 20:B10-B16. Immunopotentiating reconstituted influenza virosomes (IRIV) are used as the subunit antigen delivery system in the intranasal trivalent INFLEXAL™ product {Mischler & Metcalfe (2002) Vaccine 20 Suppl 5:B17-23} and the INFLUVAC PLUS™ product.

### E. Bacterial or Microbial Derivatives

Adjuvants suitable for use in the invention include bacterial or microbial derivatives such as:

### (1) Non-Toxic Derivatives of Enterobacterial Lipopolysaccharide (LPS)

Such derivatives include Monophosphoryl lipid A (MPL) and 3-O-deacylated MPL (3dMPL). 3dMPL is a mixture of 3 De-O-acylated monophosphoryl lipid A with 4, 5 or 6 acylated chains. A preferred “small particle” form of 3 De-O-acylated monophosphoryl lipid A is disclosed in EP 0 689 454. Such “small particles” of 3dMPL are small enough to be sterile filtered through a 0.22 micron membrane (see EP 0 689 454). Other non-toxic LPS derivatives include monophosphoryl lipid A mimics, such as aminoalkyl glucosaminide phosphate derivatives e.g. RC-529. See Johnson et al. (1999) Bioorg Med Chem Lett 9:2273-2278.

### (2) Lipid A Derivatives

Lipid A derivatives include derivatives of lipid A from Escherichia coli such as OM-174. OM-174 is described for example in Meraldi et al., “OM-174, a New Adjuvant with a Potential for Human Use, Induces a Protective Response with Administered with the Synthetic C-Terminal Fragment 242-310 from the circumsporozoite protein of Plasmodium berghei”, Vaccine (2003) 21:2485-2491; and Pajak, et al., “The Adjuvant OM-174 induces both the migration and maturation of murine dendritic cells in vivo”, Vaccine (2003) 21:836-842.

### (3) Immunostimulatory Oligonucleotides

Immunostimulatory oligonucleotides suitable for use as adjuvants in the invention include nucleotide sequences containing a CpG motif (a sequence containing an unmethylated cytosine followed by guanosine and linked by a phosphate bond). Bacterial double stranded RNA or oligonucleotides containing palindromic or poly(dG) sequences have also been shown to be immunostimulatory.

The CpG's can include nucleotide modifications/analogs such as phosphorothioate modifications and can be double-stranded or single-stranded. Optionally, the guanosine may be replaced with an analog such as 2′-deoxy-7-deazaguanosine. See Kandimalla, et al., “Divergent synthetic nucleotide motif recognition pattern: design and development of potent immunomodulatory oligodeoxyribonucleotide agents with distinct cytokine induction profiles”, Nucleic Acids Research (2003) 31(9): 2393-2400; WO02/26757 and WO99/62923 for examples of possible analog substitutions. The adjuvant effect of CpG oligonucleotides is further discussed in Krieg, “CpG motifs: the active ingredient in bacterial extracts?”, Nature Medicine (2003) 9(7): 831-835; McCluskie, et al., “Parenteral and mucosal prime-boost immunization strategies in mice with hepatitis B surface antigen and CpG DNA”, FEMS Immunology and Medical Microbiology (2002) 32:179-185; WO98/40100; U.S. Pat. No. 6,207,646; U.S. Pat. No. 6,239,116 and U.S. Pat. No. 6,429,199.

The CpG sequence may be directed to TLR9, such as the motif GTCGTT or TTCGTT. See Kandimalla, et al., “Toll-like receptor 9: modulation of recognition and cytokine induction by novel synthetic CpG DNAs”, Biochemical Society Transactions (2003) 31 (part 3): 654-658. The CpG sequence may be specific for inducing a Th1 immune response, such as a CpG-A ODN, or it may be more specific for inducing a B cell response, such a CpG-B ODN. CpG-A and CpG-B ODNs are discussed in Blackwell, et al., “CpG-A-Induced Monocyte IFN-gamma-Inducible Protein-10 Production is Regulated by Plasmacytoid Dendritic Cell Derived IFN-alpha”, J. Immunol. (2003) 170(8):4061-4068; Krieg, “From A to Z on CpG”, TRENDS in Immunology (2002) 23(2): 64-65 and WO01/95935. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, Kandimalla, et al., “Secondary structures in CpG oligonucleotides affect immunostimulatory activity”, BBRC (2003) 306:948-953; Kandimalla, et al., “Toll-like receptor 9: modulation of recognition and cytokine induction by novel synthetic GpG DNAs”, Biochemical Society Transactions (2003) 31(part 3):664-658′ Bhagat et al., “CpG penta- and hexadeoxyribonucleotides as potent immunomodulatory agents” BBRC (2003) 300:853-861 and WO03/035836.

### (4) ADP-Ribosylating Toxins and Detoxified Derivatives Thereof

Bacterial ADP-ribosylating toxins and detoxified derivatives thereof may be used as adjuvants in the invention. Preferably, the protein is derived from E. coli (i.e., E. coli heat labile enterotoxin “LT), cholera (“CT”), or pertussis (“PT”). The use of detoxified ADP-ribosylating toxins as mucosal adjuvants is described in WO95/17211 and as parenteral adjuvants in WO98/42375. Preferably, the adjuvant is a detoxified LT mutant such as LT-K63, LT-R72, and LTR192G. The use of ADP-ribosylating toxins and detoxified derivatives thereof, particularly LT-K63 and LT-R72, as adjuvants can be found in the following references: Beignon, et al., “The LTR72Mutant of Heat-Labile Enterotoxin of Escherichia coli Enhances the Ability of Peptide Antigens to Elicit CD4+ T Cells and Secrete Gamma Interferon after Coapplication onto Bare Skin”, Infection and Immunity (2002) 70(6):3012-3019; Pizza, et al., “Mucosal vaccines: non toxic derivatives of LT and CT as mucosal adjuvants”, Vaccine (2001) 19:2534-2541; Pizza, et al., “LTK63 and LTR72, two mucosal adjuvants ready for clinical trials” Int. J. Med. Microbiol. (2000) 290(4-5):455-461; Scharton-Kersten et al., “Transcutaneous Immunization with Bacterial ADP-Ribosylating Exotoxins, Subunits and Unrelated Adjuvants”, Infection and Immunity (2000) 68(9):5306-5313′ Ryan et al., “Mutants of Escherichia coli Heat-Labile Toxin Act as Effective Mucosal Adjuvants for Nasal Delivery of an Acellular Pertussis Vaccine: Differential Effects of the Nontoxic AB Complex and Enzyme Activity on Th1 and Th2 Cells” Infection and Immunity (1999) 67(12):6270-6280; Partidos et al., “Heat-labile enterotoxin of Escherichia coli and its site-directed mutant LTK63 enhance the proliferative and cytotoxic T-cell responses to intranasally co-immunized synthetic peptides”, Immunol. Lett. (1999) 67(3):209-216; Peppoloni et al., “Mutants of the Escherichia coli heat-labile enterotoxin as safe and strong adjuvants for intranasal delivery of vaccines”, Vaccines (2003) 2(2):285-293; and Pine et al., (2002) “Intranasal immunization with influenza vaccine and a detoxified mutant of heat labile enterotoxin from Escherichia coli (LTK63)” J. Control Release (2002) 85(1-3):263-270. Numerical reference for amino acid substitutions is preferably based on the alignments of the A and B subunits of ADP-ribosylating toxins set forth in Domenighini et al., Mol. Microbiol. (1995) 15(6):1165-1167.

### F. Bioadhesives and Mucoadhesives

Bioadhesives and mucoadhesives may also be used as adjuvants in the invention. Suitable bioadhesives include esterified hyaluronic acid microspheres (Singh et al. (2001) J. Cont. Rele. 70:267-276) or mucoadhesives such as cross-linked derivatives of polyacrylic acid, polyvinyl alcohol, polyvinyl pyrollidone, polysaccharides and carboxymethylcellulose. Chitosan and derivatives thereof may also be used as adjuvants in the invention. E.g. WO99/27960.

### G. Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

### H. Liposomes

Examples of liposome formulations suitable for use as adjuvants are described in U.S. Pat. No. 6,090,406, U.S. Pat. No. 5,916,588, and EP 0 626 169.

I. Polyoxyethylene ether and Polyoxyethylene Ester Formulations

Adjuvants suitable for use in the invention include polyoxyethylene ethers and polyoxyethylene esters. WO99/52549. Such formulations further include polyoxyethylene sorbitan ester surfactants in combination with an octoxynol (WO01/21207) as well as polyoxyethylene alkyl ethers or ester surfactants in combination with at least one additional non-ionic surfactant such as an octoxynol (WO01/21152).

Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

### J. Polyphosphazene (PCPP)

PCPP formulations are described, for example, in Andrianov et al., “Preparation of hydrogel microspheres by coacervation of aqueous polyphophazene solutions”, Biomaterials (1998) 19(1-3):109-115 and Payne et al., “Protein Release from Polyphosphazene Matrices”, Adv. Drug. Delivery Review (1998) 31(3):185-196.

### K. Muramyl Peptides

Examples of muramyl peptides suitable for use as adjuvants in the invention include N-acetyl-muramyl-L-threonyl-D-isoglutamine (thr-MDP), N-acetyl-normuramyl-1-alanyl-d-isoglutamine (nor-MDP), and N-acetylmuramyl-1-alanyl-d-isoglutaminyl-1-alanine-2-(1′-2′-dipalmitoyl-sn-glycero-3-hydroxyphosphoryloxy)-ethylamine MTP-PE).

L. 2-H and 2-alkyl Imidazoquinoline Compounds.

Examples of 2-H and 2-alkyl imidazoquinoline compounds suitable for use adjuvants in the invention include Imiquimod and its analogues, described further in Stanley, “Imiquimod and the imidazoquinolines: mechanism of action and therapeutic potential” Clin Exp Dermatol (2002) 27(7):571-577; Jones, “Resiquimod 3M”, Curr Opin Investig Drugs (2003) 4(2):214-218; and U.S. Pat. Nos. 4,689,338, 5,389,640, 5,268,376, 4,929,624, 5,266,575, 5,352,784, 5,494,916, 5,482,936, 5,346,905, 5,395,937, 5,238,944, 6,083,505, and 5,525,612.

### M. Thiosemicarbazone Compounds.

Examples of thiosemicarbazone compounds, as well as methods of formulating, manufacturing, and screening for compounds all suitable for use as adjuvants in the invention include those described in WO04/60308. The thiosemicarbazones are particularly effective in the stimulation of human peripheral blood mononuclear cells for the production of cytokines, such as TNF-α.

### N. Tryptanthrin Compounds.

Examples of tryptanthrin compounds, as well as methods of formulating, manufacturing, and screening for compounds all suitable for use as adjuvants in the invention include those described in WO04/64759. The tryptanthrin compounds are particularly effective in the stimulation of human peripheral blood mononuclear cells for the production of cytokines, such as TNF-α.

The invention may also comprise combinations of aspects of one or more of the adjuvants identified above. For example, the following adjuvant compositions may be used in the invention:

(1) a saponin and an oil-in-water emulsion (WO99/11241);
(2) a saponin (e.g., QS21)+a non-toxic LPS derivative (e.g. 3dMPL) (see WO94/00153);
(3) a saponin (e.g., QS21)+a non-toxic LPS derivative (e.g. 3dMPL)+a cholesterol;
(4) a saponin (e.g. QS21)+3dMPL+IL-12 (optionally+a sterol) (WO98/57659);
(5) combinations of 3dMPL with, for example, QS21 and/or oil-in-water emulsions (See European patent applications 0835318, 0735898 and 0761231);
(6) SAF, containing 10% Squalane, 0.4% Tween 80, 5% pluronic-block polymer L121, and thr-MDP, either microfluidized into a submicron emulsion or vortexed to generate a larger particle size emulsion.
(7) Ribi™ adjuvant system (RAS), (Ribi Immunochem) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and
(8) one or more mineral salts (such as an aluminum salt)+a non-toxic derivative of LPS (such as 3dPML).
(9) one or more mineral salts (such as an aluminum salt)+an immunostimulatory oligonucleotide (such as a nucleotide sequence including a CpG motif).

### O. Human Immunomodulators

Human immunomodulators suitable for use as adjuvants in the invention include cytokines, such as interleukins (e.g. IL-1, IL-2, IL-4, IL-5, IL-6, IL-7, IL-12, etc.), interferons (e.g. interferon-γ), macrophage colony stimulating factor, and tumor necrosis factor.

Aluminum salts and MF59 are preferred adjuvants for use with injectable influenza vaccines. Bacterial toxins and bioadhesives are preferred adjuvants for use with mucosally-delivered vaccines, such as nasal vaccines.

### Antigens:

Compositions of the invention may be administered in conjunction with one or more antigens for use in therapeutic, prophylactic, or diagnostic methods of the present invention. Preferred antigens include those listed below. Additionally, the compositions of the present invention may be used to treat or prevent infections caused by any of the below-listed pathogens. In addition to combination with the antigens described below, the compositions of the invention may also be combined with an adjuvant as described herein.

Antigens for use with the invention include, but are not limited to, one or more of the following antigens set forth below, or antigens derived from one or more of the pathogens set forth below:

### A. Bacterial Antigens

Bacterial antigens suitable for use in the invention include proteins, polysaccharides, lipopolysaccharides, and outer membrane vesicles which may be isolated, purified or derived from a bacteria. In addition, bacterial antigens may include bacterial lysates and inactivated bacteria formulations. Bacteria antigens may be produced by recombinant expression. Bacterial antigens preferably include epitopes which are exposed on the surface of the bacteria during at least one stage of its life cycle. Bacterial antigens are preferably conserved across multiple serotypes. Bacterial antigens include antigens derived from one or more of the bacteria set forth below as well as the specific antigens examples identified below.

Neisseria meningitides: Meningitides antigens may include proteins (such as those identified in References 1-7), saccharides (including a polysaccharide, oligosaccharide or lipopolysaccharide), or outer-membrane vesicles (References 8, 9, 10, 11) purified or derived from N. meningitides serogroup such as A, C, W135, Y, and/or B. Meningitides protein antigens may be selected from adhesions, autotransporters, toxins, Fe acquisition proteins, and membrane associated proteins (preferably integral outer membrane protein).

Streptococcus pneumoniae: Streptococcus pneumoniae antigens may include a saccharide (including a polysaccharide or an oligosaccharide) and/or protein from Streptococcus pneumoniae. Saccharide antigens may be selected from serotypes 1, 2, 3, 4, 5, 6B, 7F, 8, 9N, 9V, 10A, 11A, 12F, 14, 15B, 17F, 18C, 19A, 19F, 20, 22F, 23F, and 33F. Protein antigens may be selected from a protein identified in WO 98/18931, WO 98/18930, U.S. Pat. No. 6,699,703, U.S. Pat. No. 6,800,744, WO 97/43303, and WO 97/37026. Streptococcus pneumoniae proteins may be selected from the Poly Histidine Triad family (PhtX), the Choline Binding Protein family (CbpX), CbpX truncates, LytX family, LytX truncates, CbpX truncate-LytX truncate chimeric proteins, pneumolysin (Ply), PspA, PsaA, Sp128, Sp101, Sp130, Sp125 or Sp133.

Streptococcus pyogenes (Group A Streptococcus): Group A Streptococcus antigens may include a protein identified in WO 02/34771 or WO 2005/032582 (including GAS 40), fusions of fragments of GAS M proteins (including those described in WO 02/094851, and Dale, Vaccine (1999) 17:193-200, and Dale, Vaccine 14(10): 944-948), fibronectin binding protein (Sfb1), Streptococcal heme-associated protein (Shp), and Streptolysin S (SagA).

Moraxella catarrhalis: Moraxella antigens include antigens identified in WO 02/18595 and WO 99/58562, outer membrane protein antigens (HMW-OMP), C-antigen, and/or LPS.

Bordetella pertussis: Pertussis antigens include petussis holotoxin (PT) and filamentous haemagglutinin (FHA) from B. pertussis, optionally also combination with pertactin and/or agglutinogens 2 and 3 antigen.

Staphylococcus aureus: Staph aureus antigens include S. aureus type 5 and 8 capsular polysaccharides optionally conjugated to nontoxic recombinant Pseudomonas aeruginosa exotoxin A, such as StaphVAX™, or antigens derived from surface proteins, invasins (leukocidin, kinases, hyaluronidase), surface factors that inhibit phagocytic engulfment (capsule, Protein A), carotenoids, catalase production, Protein A, coagulase, clotting factor, and/or membrane-damaging toxins (optionally detoxified) that lyse eukaryotic cell membranes (hemolysins, leukotoxin, leukocidin).

Staphylococcus epidermis: S. epidermidis antigens include slime-associated antigen (SAA).

Clostridium tetani (Tetanus): Tetanus antigens include tetanus toxoid (TT), preferably used as a carrier protein in conjunction/conjugated with the compositions of the present invention.

Cornynebacterium diphtheriae (Diphtheria): Diphtheria antigens include diphtheria toxin, preferably detoxified, such as CRM₁₉₇. Additionally antigens capable of modulating, inhibiting or associated with ADP ribosylation are contemplated for combination/co-administration/conjugation with the compositions of the present invention. The diphtheria toxoids may be used as carrier proteins.

Haemophilus influenzae B (Hib): Hib antigens include a Hib saccharide antigen.

Pseudomonas aeruginosa: Pseudomonas antigens include endotoxin A, Wzz protein, P. aeruginosa LPS, more particularly LPS isolated from PAO1 (O5 serotype), and/or Outer Membrane Proteins, including Outer Membrane Proteins F (OprF) (Infect Immun. 2001 May; 69(5): 3510-3515).

Legionella pneumophila. Bacterial antigens may be derived from Legionella pneumophila.

Streptococcus agalactiae (Group B Streptococcus): Group B Streptococcus antigens include a protein or saccharide antigen identified in WO 02/34771, WO 03/093306, WO 04/041157, or WO 2005/002619 (including proteins GBS 80, GBS104, GBS 276 and GBS 322, and including saccharide antigens derived from serotypes Ia, Ib, Ia/c, II, III, IV, V, VI, VII and VIII).

Neiserria gonorrhoeae: Gonorrhoeae antigens include Por (or porin) protein, such as PorB (see Zhu et al., Vaccine (2004) 22:660-669), a transferring binding protein, such as TbpA and TbpB (See Price et al., Infection and Immunity (2004) 71(1):277-283), a opacity protein (such as Opa), a reduction-modifiable protein (Rmp), and outer membrane vesicle (OMV) preparations (see Plante et al., J Infectious Disease (2000) 182:848-855), also see e.g. WO99/24578, WO99/36544, WO99/57280, WO002/079243).

Chlamydia trachomatis: Chlamydia trachomatis antigens include antigens derived from serotypes A, B, Ba and C (agents of trachoma, a cause of blindness), serotypes L₁, L₂ & L₃ (associated with Lymphogranuloma venereum), and serotypes, D-K. Chlamydia trachomas antigens may also include an antigen identified in WO 00/37494, WO 03/049762, WO 03/068811, or WO 05/002619, including PepA (CT045), LcrE (CT089), ArtJ (CT381), DnaK (CT396), CT398, OmpH-like (CT242), L7/L12 (CT316), OmcA (CT444), AtosS (CT467), CT547, Eno (CT587), HrtA (CT823), and MurG (CT761).

Treponena pallidum (Syphilis): Syphilis antigens include TmpA antigen.

Haemophilus ducreyi (causing chancroid): Ducreyi antigens include outer membrane protein (DsrA).

Enterococcus faecalis or Enterococcus faecium: Antigens include a trisaccharide repeat or other Enterococcus derived antigens provided in U.S. Pat. No. 6,756,361.

Helicobacter pylori: H pylori antigens include Cag, Vac, Nap, HopX, HopY and/or urease antigen.

Staphylococcus saprophyticus: Antigens include the 160 kDa hemagglutinin of S. saprophyticus antigen.

Yersinia enterocolitica Antigens include LPS (Infect Inmun. 2002 August; 70(8): 4414).

E. coli: E. coli antigens may be derived from enterotoxigenic E. coli (ETEC), enteroaggregative E. coli (EAggEC), diffusely adhering E. coli (DAEC), enteropathogenic E. coli (EPEC), and/or enterohemorrhagic E. coli (EHEC).

Bacillus anthracis (anthrax): B. anthracis antigens are optionally detoxified and may be selected from A-components (lethal factor (LF) and edema factor (EF)), both of which can share a common B-component known as protective antigen (PA).

Yersinia pestis (plague): Plague antigens include F1 capsular antigen (Infect Immun. 2003 January; 71(1)): 374-383, LPS (Infect inmnun. 1999 October; 67(10): 5395), Yersinia pestis V antigen (Infect immun. 1997 November; 65(11): 4476-4482).

Mycobacterium tuberculosis: Tuberculosis antigens include lipoproteins, LPS, BCG antigens, a fusion protein of antigen 85B (Ag85B) and/or ESAT-6 optionally formulated in cationic lipid vesicles (Infect Immun. 2004 October; 72(10): 6148), Mycobacterium tuberculosis (Mtb) isocitrate dehydrogenase associated antigens (Proc Natl Acad Sci U S A. 2004 Aug. 24; 101(34): 12652), and/or MPT51 antigens (Infect Immun. 2004 July; 72(7): 3829).

Rickettsia: Antigens include outer membrane proteins, including the outer membrane protein A and/or B (OmpB) (Biochim Biophys Acta. 2004 Nov. 1; 1702(2):145), LPS, and surface protein antigen (SPA) (J Autoimmun. 1989 June; 2 Suppl:81).

Listeria monocytogenes. Bacterial antigens may be derived from Listeria monocytogenes.

Chlamydia pneumoniae: Antigens include those identified in WO 02/02606.

Vibrio cholerae: Antigens include proteinase antigens, LPS, particularly lipopolysaccharides of Vibrio cholerae II, O1 Inaba O-specific polysaccharides, V. cholera O139, antigens of IEM108 vaccine (Infect linmun. 2003 October; 71(10):5498-504), and/or Zonula occludens toxin (Zot).

Salmonella typhi (typhoid fever): Antigens include capsular polysaccharides preferably conjugates (Vi, i.e. vax-TyVi).

Borrelia burgdorferi (Lyme disease): Antigens include lipoproteins (such as OspA, OspB, Osp C and Osp D), other surface proteins such as OspE-related proteins (Erps), decorin-binding proteins (such as DbpA), and antigenically variable VI proteins, such as antigens associated with P39 and P13 (an integral membrane protein, Infect Immun. 2001 May; 69(5): 3323-3334), VlsE Antigenic Variation Protein (J Clin Microbiol. 1999 December; 37(12): 3997).

Porphyromonas gingivalis: Antigens include P. gingivalis outer membrane protein (OMP).

Klebsiella: Antigens include an OMP, including OMP A, or a polysaccharide optionally conjugated to tetanus toxoid.

Further bacterial antigens of the invention may be capsular antigens, polysaccharide antigens or protein antigens of any of the above. Further bacterial antigens may also include an outer membrane vesicle (OMV) preparation. Additionally, antigens include live, attenuated, and/or purified versions of any of the aforementioned bacteria. The antigens of the present invention may be derived from gram-negative or gram-positive bacteria. The antigens of the present invention may be derived from aerobic or anaerobic bacteria.

Additionally, any of the above bacterial-derived saccharides (polysaccharides, LPS, LOS or oligosaccharides) can be conjugated to another agent or antigen, such as a carrier protein (for example CRM₁₉₇). Such conjugation may be direct conjugation effected by reductive amination of carbonyl moieties on the saccharide to amino groups on the protein, as provided in U.S. Pat. No. 5,360,897 and Can J Biochem Cell Biol. 1984 May; 62(5):270-5. Alternatively, the saccharides can be conjugated through a linker, such as, with succinamide or other linkages provided in Bioconjugate Techniques, 1996 and CRC, Chemistry of Protein Conjugation and Cross-Linking, 1993.

### B. Viral Antigens

Viral antigens suitable for use in the invention include inactivated (or killed) virus, attenuated virus, split virus formulations, purified subunit formulations, viral proteins which may be isolated, purified or derived from a virus, and Virus Like Particles (VLPs). Viral antigens may be derived from viruses propagated on cell culture or other substrate. Alternatively, viral antigens may be expressed recombinantly. Viral antigens preferably include epitopes which are exposed on the surface of the virus during at least one stage of its life cycle. Viral antigens are preferably conserved across multiple serotypes or isolates. Viral antigens include antigens derived from one or more of the viruses set forth below as well as the specific antigens examples identified below.

Orthomnyxovirus: Viral antigens may be derived from an Orthomyxovirus, such as Influenza A, B and C. Orthomyxovirus antigens may be selected from one or more of the viral proteins, including hemagglutinin (HA), neuraminidase (NA), nucleoprotein (NP), matrix protein (M1), membrane protein (M2), one or more of the transcriptase components (PB1, PB2 and PA). Preferred antigens include HA and NA.

Influenza antigens may be derived from interpandemic (annual) flu strains. Alternatively influenza antigens may be derived from strains with the potential to cause pandemic a pandemic outbreak (i.e., influenza strains with new haemagglutinin compared to the haemagglutinin in currently circulating strains, or influenza strains which are pathogenic in avian subjects and have the potential to be transmitted horizontally in the human population, or influenza strains which are pathogenic to humans).

Paramnyxoviridae viruses: Viral antigens may be derived from Paramyxoviridae viruses, such as Pneumoviruses (RSV), Paramyxoviruses (PIV) and Morbilliviruses (Measles).

Pneumovirus: Viral antigens may be derived from a Pneumovirus, such as Respiratory syncytial virus (RSV), Bovine respiratory syncytial virus, Pneumonia virus of mice, and Turkey rhinotracheitis virus. Preferably, the Pneumovirus is RSV. Pneumovirus antigens may be selected from one or more of the following proteins, including surface proteins Fusion (F), Glycoprotein (G) and Small Hydrophobic protein (SH), matrix proteins M and M2, nucleocapsid proteins N, P and L and nonstructural proteins NS1 and NS2. Preferred Pneumovirus antigens include F, G and M. See e.g., J Gen Virol. 2004 November; 85(Pt 11):3229). Pneumovirus antigens may also be formulated in or derived from chimeric viruses. For example, chimeric RSV/PIV viruses may comprise components of both RSV and PIV.

Paramyxovirus: Viral antigens may be derived from a Paramyxovirus, such as Parainfluenza virus types 1-4 (PIV), Mumps, Sendai viruses, Simian virus 5, Bovine parainfluenza virus and Newcastle disease virus. Preferably, the Paramyxovirus is PIV or Mumps. Paramyxovirus antigens may be selected from one or more of the following proteins: Hemagglutinin-Neuraminidase (HN), Fusion proteins F1 and F2, Nucleoprotein (NP), Phosphoprotein (P), Large protein (L), and Matrix protein (M). Preferred Paramyxovirus proteins include HN, F1 and F2. Paramyxovirus antigens may also be formulated in or derived from chimeric viruses. For example, chimeric RSV/PIV viruses may comprise components of both RSV and PIV. Commercially available mumps vaccines include live attenuated mumps virus, in either a monovalent form or in combination with measles and rubella vaccines (MMR).

Morbillivirus: Viral antigens may be derived from a Morbillivirus, such as Measles. Morbillivirus antigens may be selected from one or more of the following proteins: hemagglutinin (H), Glycoprotein (G), Fusion factor (F), Large protein (L), Nucleoprotein (NP), Polymerase phosphoprotein (P), and Matrix (M). Commercially available measles vaccines include live attenuated measles virus, typically in combination with mumps and rubella (MMR).

Picornavirus: Viral antigens may be derived from Picornaviruses, such as Enteroviruses, Rhinoviruses, Heparnavirus, Cardioviruses and Aphthoviruses. Antigens derived from Enteroviruses, such as Poliovirus are preferred.

Enterovirus: Viral antigens may be derived from an Enterovirus, such as Poliovirus types 1, 2 or 3, Coxsackie A virus types 1 to 22 and 24, Coxsackie B virus types 1 to 6, Echovirus (ECHO) virus) types 1 to 9, 11 to 27 and 29 to 34 and Enterovirus 68 to 71. Preferably, the Enterovirus is poliovirus. Enterovirus antigens are preferably selected from one or more of the following Capsid proteins VP1, VP2, VP3 and VP4. Commercially available polio vaccines include Inactivated Polio Vaccine (IPV) and Oral poliovirus vaccine (OPV).

Heparnavirus: Viral antigens may be derived from an Hepamavirus, such as Hepatitis A virus (HAV). Commercially available HAV vaccines include inactivated HAV vaccine.

Togavirus: Viral antigens may be derived from a Togavirus, such as a Rubivirus, an Alphavirus, or an Arterivirus. Antigens derived from Rubivirus, such as Rubella virus, are preferred. Togavirus antigens may be selected from E1, E2, E3, C, NSP-1, NSPO-2, NSP-3 or NSP-4. Togavirus antigens are preferably selected from E1, E2 or E3. Commercially available Rubella vaccines include a live cold-adapted virus, typically in combination with mumps and measles vaccines (MMR).

Flavivirus: Viral antigens may be derived from a Flavivirus, such as Tick-borne encephalitis (TBE), Dengue (types 1, 2, 3 or 4), Yellow Fever, Japanese encephalitis, West Nile encephalitis, St. Louis encephalitis, Russian spring-summer encephalitis, Powassan encephalitis. Flavivirus antigens may be selected from PrM, M, C, E, NS-1, NS-2a, NS2b, NS3, NS4a, NS4b, and NS5. Flavivirus antigens are preferably selected from PrM, M and E. Commercially available TBE vaccine include inactivated virus vaccines.

Pestivirus: Viral antigens may be derived from a Pestivirus, such as Bovine viral diarrhea (BVDV), Classical swine fever (CSFV) or Border disease (BDV).

Hepadnavirus: Viral antigens may be derived from a Hepadnavirus, such as Hepatitis B virus. Hepadnavirus antigens may be selected from surface antigens (L, M and S), core antigens (HBc, HBe). Commercially available HBV vaccines include subunit vaccines comprising the surface antigen S protein.

Hepatitis C virus: Viral antigens may be derived from a Hepatitis C virus (HCV). HCV antigens may be selected from one or more of E1, E2, E1/E2, NS345 polyprotein, NS 345-core polyprotein, core, and/or peptides from the nonstructural regions (Houghton et al., Hepatology (1991) 14:381).

Rhabdovirus: Viral antigens may be derived from a Rhabdovirus, such as a Lyssavirus (Rabies virus) and Vesiculovirus (VSV). Rhabdovirus antigens may be selected from glycoprotein (G), nucleoprotein (N), large protein (L), nonstructural proteins (NS). Commercially available Rabies virus vaccine comprise killed virus grown on human diploid cells or fetal rhesus lung cells.

Caliciviridae: Viral antigens may be derived from Calciviridae, such as Norwalk virus, and Norwalk-like Viruses, such as Hawaii Virus and Snow Mountain Virus.

Coronavirus: Viral antigens may be derived from a Coronavirus, SARS, Human respiratory coronavirus, Avian infectious bronchitis (IBV), Mouse hepatitis virus (MHV), and Porcine transmissible gastroenteritis virus (TGEV). Coronavirus antigens may be selected from spike (S), envelope (E), matrix (M), nucleocapsid (N), and Hemagglutinin-esterase glycoprotein (HE). Preferably, the Coronavirus antigen is derived from a SARS virus. SARS viral antigens are described in WO 04/92360;

Retrovirus: Viral antigens may be derived from a Retrovirus, such as an Oncovirus, a Lentivirus or a Spumavirus. Oncovirus antigens may be derived from HTLV-1, HTLV-2 or HTLV-5. Lentivirus antigens may be derived from HIV-1 or HIV-2. Retrovirus antigens may be selected from gag, pol, env, tax, tat, rex, rev, nef, vif, vpu, and vpr. HIV antigens may be selected from gag (p24gag and p55gag), env (gp160 and gp41), pol, tat, nef, rev vpu, miniproteins, (preferably p55 gag and gp140v delete). HIV antigens may be derived from one or more of the following strains: HIV_{IIIb}, HIV_{SF2}, HIV_{LAV}, HIV_{LAI}, HIV_{MN}, HIV-1_{CM235}, HIV-1_{US4}.

Reovirus: Viral antigens may be derived from a Reovirus, such as an Orthoreovirus, a Rotavirus, an Orbivirus, or a Coltivirus. Reovirus antigens may be selected from structural proteins λ1, λ2, λ3, μ1, μ2, σ1, σ2, or σ3, or nonstructural proteins σNS, μNS, or σ1. Preferred Reovirus antigens may be derived from a Rotavirus. Rotavirus antigens may be selected from VP1, VP2, VP3, VP4 (or the cleaved product VP5 and VP8), NSP 1, VP6, NSP3, NSP2, VP7, NSP4, or NSP5. Preferred Rotavirus antigens include VP4 (or the cleaved product VP5 and VP8), and VP7.

Parvovirus: Viral antigens may be derived from a Parvovirus, such as Parvovirus B19. Parvovirus antigens may be selected from VP-1, VP-2, VP-3, NS-1 and NS-2. Preferably, the Parvovirus antigen is capsid protein VP-2.

Delta hepatitis virus (HDV): Viral antigens may be derived HDV, particularly 8-antigen from HDV (see, e.g., U.S. Pat. No. 5,378,814).

Hepatitis E virus (HEV): Viral antigens may be derived from HEV.

Hepatitis G virus (HGV): Viral antigens may be derived from HGV.

Human Herpesvirus: Viral antigens may be derived from a Human Herpesvirus, such as Herpes Simplex Viruses (HSV), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8). Human Herpesvirus antigens may be selected from immediate early proteins (α), early proteins (β), and late proteins (γ). HSV antigens may be derived from HSV-1 or HSV-2 strains. HSV antigens may be selected from glycoproteins gB, gC, gD and gH, fusion protein (gB), or immune escape proteins (gC, gE, or gI). VZV antigens may be selected from core, nucleocapsid, tegument, or envelope proteins. A live attenuated VZV vaccine is commercially available. EBV antigens may be selected from early antigen (EA) proteins, viral capsid antigen (VCA), and glycoproteins of the membrane antigen (MA). CMV antigens may be selected from capsid proteins, envelope glycoproteins (such as gB and gH), and tegument proteins

Papovaviruses: Antigens may be derived from Papovaviruses, such as Papillomaviruses and Polyomaviruses. Papillomaviruses include HPV serotypes 1, 2, 4, 5, 6, 8, 11, 13, 16, 18, 31, 33, 35, 39, 41, 42, 47, 51, 57, 58, 63 and 65. Preferably, HPV antigens are derived from serotypes 6, 11, 16 or 18. HPV antigens may be selected from capsid proteins (L1) and (L2), or E1-E7, or fusions thereof. HPV antigens are preferably formulated into virus-like particles (VLPs). Polyomyavirus viruses include BK virus and JK virus. Polyomavirus antigens may be selected from VP1, VP2 or VP3.

Further provided are antigens, compositions, methods, and microbes included in Vaccines, 4^{th} Edition (Plotkin and Orenstein ed. 2004); Medical Microbiology 4^{th} Edition (Murray et al. ed. 2002); Virology, 3rd Edition (W. K. Joklik ed. 1988); Fundamental Virology, 2nd Edition (B. N. Fields and D. M. Knipe, eds. 1991), which are contemplated in conjunction with the compositions of the present invention.

### C. Fungal Antigens

Fungal antigens for use in the invention may be derived from one or more of the fungi set forth below.

Fungal antigens may be derived from Dermatophytres, including: Epidermophyton floccusum, Microsporum audouini, Microsporum canis, Microsporum distortum, Microsporunm equinum, Microsporum gypsum, Microsporumn nanumn, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var. album, var. discoides, var. ochraceum, Trichophyton violaceum, and/or Trichophyton faviforme.

Fungal pathogens may be derived from Aspergillus fumigatus, Aspergillus flavus, Aspergillus niger, Aspergillus nidulans, Aspergillus terreus, Aspergillus sydowi, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatunz, Histoplasma capsulatum, Klebsiella pneumoniae, Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

Processes for producing a fungal antigens are well known in the art (see U.S. Pat. No. 6,333,164). In a preferred method a solubilized fraction extracted and separated from an insoluble fraction obtainable from fungal cells of which cell wall has been substantially removed or at least partially removed, characterized in that the process comprises the steps of: obtaining living fungal cells; obtaining fungal cells of which cell wall has been substantially removed or at least partially removed; bursting the fungal cells of which cell wall has been substantially removed or at least partially removed; obtaining an insoluble fraction; and extracting and separating a solubilized fraction from the insoluble fraction.

### D. STD Antigens

The compositions of the invention may include one or more antigens derived from a sexually transmitted disease (STD). Such antigens may provide for prophylactis or therapy for STD's such as chlamydia, genital herpes, hepatits (such as HCV), genital warts, gonorrhoea, syphilis and/or chancroid (See, WO00/15255). Antigens may be derived from one or more viral or bacterial STD's. Viral STD antigens for use in the invention may be derived from, for example, HIV, herpes simplex virus (HSV-1 and HSV-2), human papillomavirus (HPV), and hepatitis (HCV). Bacterial STD antigens for use in the invention may be derived from, for example, Neiserria gonorrhoeae, Chlamydia trachomatis, Treponema pallidum, Haemophilus ducreyi, E. coli, and Streptococcus agalactiae. Examples of specific antigens derived from these pathogens are described above.

### E. Respiratory Antigens

The compositions of the invention may include one or more antigens derived from a pathogen which causes respiratory disease. For example, respiratory antigens may be derived from a respiratory virus such as Orthomyxoviruses (influenza), Pneumovirus (RSV), Paramyxovirus (PIV), Morbillivirus (measles), Togavirus (Rubella), VZV, and Coronavirus (SARS). Respiratory antigens may be derived from a bacteria which causes respiratory disease, such as Streptococcus pneumoniae, Pseudomonas aeruginosa, Bordetella pertussis, Mycobacterium tuberculosis, Mycoplasma pneumoniae, Chlamydia pneumoniae, Bacillus anthracis, and Moraxella catarrhalis. Examples of specific antigens derived from these pathogens are described above.

### F. Pediatric Vaccine Antigens

The compositions of the invention may include one or more antigens suitable for use in pediatric subjects. Pediatric subjects are typically less than about 3 years old, or less than about 2 years old, or less than about 1 years old. Pediatric antigens may be administered multiple times over the course of 6 months, 1, 2 or 3 years. Pediatric antigens may be derived from a virus which may target pediatric populations and/or a virus from which pediatric populations are susceptible to infection. Pediatric viral antigens include antigens derived from one or more of Orthomyxovirus (influenza), Pneumovirus (RSV), Paramyxovirus (PIV and Mumps), Morbillivirus (measles), Togavirus (Rubella), Enterovirus (polio), HBV, Coronavirus (SARS), and Varicella-zoster virus (VZV), Epstein Barr virus (EBV). Pediatric bacterial antigens include antigens derived from one or more of Streptococcus pneumoniae, Neisseria meningitides, Streptococcus pyogenes (Group A Streptococcus), Moraxella catarrhalis, Bordetella pertussis, Staphylococcus aureus, Clostridium tetani (Tetanus), Cornynebacterium diphtheriae (Diphtheria), Haemophilus influenzae B (Hib), Pseudomonas aeruginosa, Streptococcus agalactiae (Group B Streptococcus), and E. coli. Examples of specific antigens derived from these pathogens are described above.

### G. Antigens Suitable for Use in Elderly or Immunocompromised Individuals

The compositions of the invention may include one or more antigens suitable for use in elderly or immunocompromised individuals. Such individuals may need to be vaccinated more frequently, with higher doses or with adjuvanted formulations to improve their immune response to the targeted antigens. Antigens which may be targeted for use in Elderly or Immunocompromised individuals include antigens derived from one or more of the following pathogens: Neisseria meningitides, Streptococcus pneumoniae, Streptococcus pyogenes (Group A Streptococcus), Moraxella catarrhalis, Bordetella pertussis, Staphylococcus aureus, Staphylococcus epidermis, Clostridium tetani (Tetanus), Cornynebacterium diphtheriae (Diphtheria), Haemophilus influenzae B (Hib), Pseudomonas aeruginosa, Legionella pneumophila, Streptococcus agalactiae (Group B Streptococcus), Enterococcus faecalis, Helicobacter pylori, Clamydia pneumoniae, Orthomyxovirus (influenza), Pneumovirus (RSV), Paramyxovirus (PIV and Mumps), Morbillivirus (measles), Togavirus (Rubella), Enterovirus (polio), HBV, Coronavirus (SARS), Varicella-zoster virus (VZV), Epstein Barr virus (EBV), Cytomegalovirus (CMV). Examples of specific antigens derived from these pathogens are described above.

### H. Antigens Suitable for Use in Adolescent Vaccines

The compositions of the invention may include one or more antigens suitable for use in adolescent subjects. Adolescents may be in need of a boost of a previously administered pediatric antigen. Pediatric antigens which may be suitable for use in adolescents are described above. In addition, adolescents may be targeted to receive antigens derived from an STD pathogen in order to ensure protective or therapeutic immunity before the beginning of sexual activity. STD antigens which may be suitable for use in adolescents are described above.

### I. Tumor Antigens

One embodiment of the present involves a tumor antigen or cancer antigen in conjunction with the compositions of the present invention. Tumor antigens can be, for example, peptide-containing tumor antigens, such as a polypeptide tumor antigen or glycoprotein tumor antigens. A tumor antigen can also be, for example, a saccharide-containing tumor antigen, such as a glycolipid tumor antigen or a ganglioside tumor antigen. The tumor antigen can further be, for example, a polynucleotide-containing tumor antigen that expresses a polypeptide-containing tumor antigen, for instance, an RNA vector construct or a DNA vector construct, such as plasmid DNA.

Tumor antigens appropriate for the practice of the present invention encompass a wide variety of molecules, such as (a) polypeptide-containing tumor antigens, including polypeptides (which can range, for example, from 8-20 amino acids in length, although lengths outside this range are also common), lipopolypeptides and glycoproteins, (b) saccharide-containing tumor antigens, including poly-saccharides, mucins, gangliosides, glycolipids and glycoproteins, and (c) polynucleotides that express antigenic polypeptides.

The tumor antigens can be, for example, (a) full length molecules associated with cancer cells, (b) homologs and modified forms of the same, including molecules with deleted, added and/or substituted portions, and (c) fragments of the same. Tumor antigens can be provided in recombinant form. Tumor antigens include, for example, class I-restricted antigens recognized by CD8+ lymphocytes or class II-restricted antigens recognized by CD4+ lymphocytes.

Numerous tumor antigens are known in the art, including: (a) cancer-testis antigens such as NY-ESO-1, SSX2, SCP1 as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors), (b) mutated antigens, for example, p53 (associated with various solid tumors, e.g., colorectal, lung, head and neck cancer), p21/Ras (associated with, e.g., melanoma, pancreatic cancer and colorectal cancer), CDK4 (associated with, e.g., melanoma), MUM1 (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT, (c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), alpha-fetoprotein (associated with, e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer), (d) shared antigens, for example, melanoma-melanocyte differentiation antigens such as MART-1/Melan A, gp100, MC1R, melanocyte-stimulating hormone receptor, tyrosinase, tyrosinase related protein-1/TRP1 and tyrosinase related protein-2/TRP2 (associated with, e.g., melanoma), (e) prostate associated antigens such as PAP, PSA, PSMA, PSH-P1, PSM-P1, PSM-P2, associated with e.g., prostate cancer, (f) immunoglobulin idiotypes (associated with myeloma and B cell lymphomas, for example), and (g) other tumor antigens, such as polypeptide- and saccharide-containing antigens including (i) glycoproteins such as sialyl Tn and sialyl Le^{x} (associated with, e.g., breast and colorectal cancer) as well as various mucins; glycoproteins may be coupled to a carrier protein (e.g., MUC-1 may be coupled to KLH); (ii) lipopolypeptides (e.g., MUC-1 linked to a lipid moiety); (iii) polysaccharides (e.g., Globo H synthetic hexasaccharide), which may be coupled to a carrier proteins (e.g., to KLH), (iv) gangliosides such as GM2, GM12, GD2, GD3 (associated with, e.g., brain, lung cancer, melanoma), which also may be coupled to carrier proteins (e.g., KLH). Additional tumor antigens which are known in the art include p15, Hom/MeI-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, p185erbB2, p180erbB-3, c-met, mn-23H1, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29BCAA), CA 195, CA 242, CA-50, CAM43, CD68KP1, CO-029, FGF-5, Ga733 (EpCAM), HTgp-175, M344, MA-50, MG7-Ag, MOV18, NB/70K, NY—CO-1, RCAS1, SDCCAG16, TA-90 (Mac-2 binding protein\cyclophilin C-associated protein), TAAL6, TAG72, TLP, TPS, and the like. These as well as other cellular components are described for example in United States Patent Application 20020007173 and references cited therein.

Polynucleotide-containing antigens in accordance with the present invention typically comprise polynucleotides that encode polypeptide cancer antigens such as those listed above. Preferred polynucleotide-containing antigens include DNA or RNA vector constructs, such as plasmid vectors (e.g., pCMV), which are capable of expressing polypeptide cancer antigens in vivo.

Tumor antigens may be derived, for example, from mutated or altered cellular components. After alteration, the cellular components no longer perform their regulatory functions, and hence the cell may experience uncontrolled growth. Representative examples of altered cellular components include ras, p53, Rb, altered protein encoded by the Wilms' tumor gene, ubiquitin, mucin, protein encoded by the DCC, APC, and MCC genes, as well as receptors or receptor-like structures such as neu, thyroid hormone receptor, platelet derived growth factor (PDGF) receptor, insulin receptor, epidermal growth factor (EGF) receptor, and the colony stimulating factor (CSF) receptor. These as well as other cellular components are described for example in U.S. Pat. No. 5,693,522 and references cited therein.

Additionally, bacterial and viral antigens, may be used in conjunction with the compositions of the present invention for the treatment of cancer. In particular, carrier proteins, such as CRM₁₉₇, tetanus toxoid, or Salmonella typhimurium antigen can be used in conjunction/conjugation with compounds of the present invention for treatment of cancer. The cancer antigen combination therapies will show increased efficacy and bioavailability as compared with existing therapies.

Additional information on cancer or tumor antigens can be found, for example, in Moingeon P, “Cancer vaccines,” Vaccine, 2001, 19:1305-1326; Rosenberg S A, “Progress in human tumor immunology and immunotherapy,” Nature, 2001, 411:380-384; Dermine, S. et al, “Cancer Vaccines and Immunotherapy,” British Medical Bulletin, 2002, 62, 149-162; Espinoza-Delgado I., “Cancer Vaccines,” The Oncologist, 2002, 7(suppl3):20-33; Davis, I. D. et al., “Rational approaches to human cancer immunotherapy,” Journal of Leukocyte Biology, 2003, 23: 3-29; Van den Eynde B, et al., “New tumor antigens recognized by T cells,” Curr. Opin. Immunol., 1995, 7:674-81; Rosenberg S A, “Cancer vaccines based on the identification of genes encoding cancer regression antigens, Immunol. Today, 1997, 18:175-82; Offring a R et al., “Design and evaluation of antigen-specific vaccination strategies against cancer,” Current Opin. Immunol., 2000, 2:576-582; Rosenberg S A, “A new era for cancer immunotherapy based on the genes that encode cancer antigens,” Immunity, 1999, 10:281-7; Sahin U et al., “Serological identification of human tumor antigens,” Curr. Opin. Immunol., 1997, 9:709-16; Old L J et al., “New paths in human cancer serology,” J. Exp. Med., 1998, 187:1163-7; Chaux P, et al., “Identification of MAGE-3 epitopes presented by HLA-DR molecules to CD4(+) T lymphocytes,” J. Exp. Med., 1999, 189:767-78; Gold P, et al., “Specific carcinoembryonic antigens of the human digestive system,” J. Exp. Med., 1965, 122:467-8; Livingston P O, et al., Carbohydrate vaccines that induce antibodies against cancer: Rationale,” Cancer Immunol. Immunother., 1997, 45:1-6; Livingston P O, et al., Carbohydrate vaccines that induce antibodies against cancer: Previous experience and future plans,” Cancer Immunol. Immunother., 1997, 45:10-9; Taylor-Papadimitriou J, “Biology, biochemistry and immunology of carcinoma-associated mucins,” Immunol. Today, 1997, 18:105-7; Zhao X-J et al., “GD2 oligosaccharide: target for cytotoxic T lymphocytes,” J. Exp. Med., 1995, 182:67-74; Theobald M, et al., “Targeting p53 as a general tumor antigen,” Proc. Natl. Acad. Sci. USA, 1995, 92:11993-7; Gaudernack G, “T cell responses against mutant ras: a basis for novel cancer vaccines,” Immunotechnology, 1996, 2:3-9; WO 91/02062; U.S. Pat. No. 6,015,567; WO 01/08636; WO 96/30514; U.S. Pat. No. 5,846,538; and U.S. Pat. No. 5,869,445.

### J. Antigen Formulations

In other aspects of the invention, methods of producing microparticles having adsorbed antigens are provided. The methods comprise: (a) providing an emulsion by dispersing a mixture comprising (i) water, (ii) a detergent, (iii) an organic solvent, and (iv) a biodegradable polymer selected from the group consisting of a poly(α-hydroxy acid), a polyhydroxy butyric acid, a polycaprolactone, a polyorthoester, a polyanhydride, and a polycyanoacrylate. The polymer is typically present in the mixture at a concentration of about 1% to about 30% relative to the organic solvent, while the detergent is typically present in the mixture at a weight-to-weight detergent-to-polymer ratio of from about 0.00001:1 to about 0.1:1 (more typically about 0.0001:1 to about 0.1:1, about 0.001:1 to about 0.1:1, or about 0.005:1 to about 0.1:1); (b) removing the organic solvent from the emulsion; and (c) adsorbing an antigen on the surface of the microparticles. In certain embodiments, the biodegradable polymer is present at a concentration of about 3% to about 10% relative to the organic solvent.

Microparticles for use herein will be formed from materials that are sterilizable, non-toxic and biodegradable. Such materials include, without limitation, poly(α-hydroxy acid), polyhydroxybutyric acid, polycaprolactone, polyorthoester, polyanhydride, PACA, and polycyanoacrylate. Preferably, microparticles for use with the present invention are derived from a poly(α-hydroxy acid), in particular, from a poly(lactide) (“PLA”) or a copolymer of D,L-lactide and glycolide or glycolic acid, such as a poly(D,L-lactide-co-glycolide) (“PLG” or “PLGA”), or a copolymer of D,L-lactide and caprolactone. The microparticles may be derived from any of various polymeric starting materials which have a variety of molecular weights and, in the case of the copolymers such as PLG, a variety of lactide:glycolide ratios, the selection of which will be largely a matter of choice, depending in part on the coadministered macromolecule. These parameters are discussed more fully below.

Further antigens may also include an outer membrane vesicle (OMV) preparation.

Additional formulation methods and antigens (especially tumor antigens) are provided in U.S. patent Ser. No. 09/581,772.

### K. Antigen References

The following references include antigens useful in conjunction with the compositions of the present invention:

Antigen references are listed below:
1. International patent application WO 99/24578
2. International patent application WO 99/36544.
3. International patent application WO 99/57280.
4. International patent application WO 00/22430.
5. Tettelin et al. (2000) Science 287:1809-1815.
6. International patent application WO 96/29412.
7. Pizza et al. (2000) Science 287:1816-1820.
8. PCT WO 01/52885.
9. Bjune et al. (1991) Lancet 338(8775).
10. Fuskasawa et al. (1999) Vaccine 17:2951-2958.
11. Rosenqist et al. (1998) Dev. Biol. Strand 92:323-333.
12. Constantino et al. (1992) Vaccine 10:691-698.
13. Constantino et al. (1999) Vaccine 17:1251-1263.
14. Watson (2000) Pediatr Infect Dis J 19:331-332.
15. Rubin (20000) Pediatr Clin North Am 47:269-285,v.
16. Jedrzejas (2001) Microbiol Mol Biol Rev 65:187-207.
17. International patent application filed on 3 Jul. 2001 claiming priority from GB-0016363.4; WO 02/02606; PCT IB/01/00166.
18. Kalman et al. (1999) Nature Genetics 21:385-389.
19. Read et al. (2000) Nucleic Acids Res 28:1397-406.
20. Shirai et al. (2000) J. Infect. Dis 181(Suppl 3):S524-S527.
21. International patent application WO 99/27105.
22. International patent application WO 00/27994.
23. International patent application WO 00/37494.
24. International patent application WO 99/28475.
25. Bell (2000) Pediatr Infect Dis J 19:1187-1188.
26. Iwarson (1995) APMIS103:321-326.
27. Gerlich et al. (1990) Vaccine 8 Suppl:S63-68 & 79-80.
28. Hsu et al. (1999) Clin Liver Dis 3:901-915.
29. Gastofsson et al. (1996) N. Engl. J. Med. 334-:349-355.
30. Rappuoli et al. (1991) TIBTECH 9:232-238.
31. Vaccines (1988) eds. Plotkin & Mortimer. ISBN 0-7216-1946-0.
32. Del Guidice et al. (1998) Molecular Aspects of Medicine 19:1-70.
33. International patent application WO 93/018150.
34. International patent application WO 99/53310.
35. International patent application WO 98/04702.
36. Ross et al. (2001) Vaccine 19:135-142.
37. Sutter et al. (2000) Pediatr Clin North Am 47:287-308.
38. Zimmerman & Spann (1999) Am Fan Physician 59:113-118, 125-126.
39. Dreensen (1997) Vaccine 15 Suppl″S2-6.
40. MMWR Morb Mortal Wkly rep 1998 Jan. 16:47(1):12, 9.
41. McMichael (2000) Vaccine 19 Suppl 1:S101-107.
42. Schuchat (1999) Lancer 353(9146):51-6.
43. GB patent applications 0026333.5, 0028727.6 & 0105640.7.
44. Dale (1999) Infect Disclin North Am 13:227-43, viii.
45. Ferretti et al. (2001) PNAS USA 98: 4658-4663.
46. Kuroda et al. (2001) Lancet 357(9264):1225-1240; see also pages 1218-1219.
47. Ramsay et al. (2001) Lancet 357(9251):195-196.
48. Lindberg (1999) Vaccine 17 Suppl 2:S28-36.
49. Buttery & Moxon (2000) J R Coil Physicians Long 34:163-168.
50. Ahmad & Chapnick (1999) Infect Dis Clin North Am 13:113-133, vii.
51. Goldblatt (1998) J. Med. Microbiol. 47:663-567.
52. European patent 0 477 508.
53. U.S. Pat. No. 5,306,492.
54. International patent application WO 98/42721.
55. Conjugate Vaccines (eds. Cruse et al.) ISBN 3805549326, particularly vol. 10:48-114.
56. Hermanson (1996) Bioconjugate Techniques ISBN: 012323368 & 012342335X.
57. European patent application 0372501.
58. European patent application 0378881.
59. European patent application 0427347.
60. International patent application WO 93/17712.
61. International patent application WO 98/58668.
62. European patent application 0471177.
63. International patent application WO 00/56360.
64. International patent application WO 00/67161.

Pharmaceutical compositions that include the compounds described herein may include additives such as excipients. Suitable pharmaceutically acceptable excipients include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidinone, low melting waxes, ion exchange resins, and the like, as well as combinations of any two or more of these. Other suitable pharmaceutically acceptable excipients are described in “Remington's Pharmaceutical Sciences,” Mack Pub. Co., New Jersey (1991), which is hereby incorporated herein by reference in its entirety and for all purposes as if fully set forth herein.

Pharmaceutical compositions that include the compounds of the invention may be in any form suitable for the intended method of administration, including, for example, as a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more of these. The liquid carrier may include other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, but are not limited to, soybean oil, coconut oil, olive oil, safflower oil, cottonseed oil, and the like. For parenteral administration, the carrier may be an oily ester such as ethyl oleate, isopropyl myristate, and the like. Compositions of the present invention may also be in the form of microparticles, microcapsules, and the like, as well as combinations of any two or more of these.

The compounds and combinations of the present invention can also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. The present compositions in liposome form may include, in addition to a compound of the present invention, stabilizers, preservatives, excipients, and the like. Preferred lipids include phospholipids and phosphatidyl cholines (lecithins), both natural and synthetic. Methods of forming liposomes are known in the art. See, for example, Prescott, Ed., Methods in Cell Biology, Volume XIV, Academic Press, New York, N.W., p. 33 et seq (1976).

Other additives in the compositions of the invention may include immunostimulatory agents known in the art or listed herein. Immunostimulatory oligonucleotides and polynucleotides are described in PCT WO 98/55495 and PCT WO 98/16247. U.S. Patent Application No. 2002/0164341 describes adjuvants including an unmethylated CpG dinucleotide (CpG ODN) and a non-nucleic acid adjuvant. U.S. Patent Application No. 2002/0197269 describes compositions comprising an antigen, an antigenic CpG-ODN and a polycationic polymer. Other immunostimulatory additives described in the art may also be used, for example, as described in U.S. Pat. No. 5,026,546; U.S. Pat. No. 4,806,352; and U.S. Pat. No. 5,026,543. Additionally, SMIP compounds as described in U.S. Ser. No. 10/814,480 and 60/582,654 are contemplated as effective co-administration agents or may be used in combination with the compositions of the instant invention.

Controlled release delivery systems may also be used, such as a diffusion controlled matrix system or an erodible system, as described for example in: Lee, “Diffusion-Controlled Matrix Systems”, pp. 155-198 and Ron and Langer, “Erodible Systems”, pp. 199-224, in “Treatise on Controlled Drug Delivery”, A. Kydonieus Ed., Marcel Dekker, Inc., New York 1992. The matrix may be, for example, a biodegradable material that can degrade spontaneously in situ and in vivo for, example, by hydrolysis or enzymatic cleavage, e.g., by proteases. The delivery system may be, for example, a naturally occurring or synthetic polymer or copolymer, for example in the form of a hydrogel. Exemplary polymers with cleavable linkages include polyesters, polyorthoesters, polyanhydrides, polysaccharides, poly(phosphoesters), polyamides, polyurethanes, poly(imidocarbonates) and poly(phosphazenes).

The compounds of the invention may be administered enterally, orally, parenterally, sublingually, by inhalation spray, rectally, or topically in dosage unit formulations that include conventional nontoxic pharmaceutically acceptable carriers, adjuvants, and vehicles as desired. For example, suitable modes of administration include oral, subcutaneous, transdermal, transmucosal, iontophoretic, intravenous, intramuscular, intraperitoneal, intranasal, subdermal, rectal, and the like. Topical administration may also include the use of transdermal administration such as transdermal patches or ionophoresis devices. The term parenteral as used herein includes subcutaneous injections, intravenous, intramuscular, intrasternal injection, or infusion techniques.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-propanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols that are solid at ordinary temperatures but liquid at the rectal temperature and will, therefore, melt in the rectum and release the drug.

Solid dosage forms for oral administration may include capsules, tablets, pills, powders, and granules. In such solid dosage forms, the active compound may be admixed with at least one inert diluent such as sucrose lactose or starch. Such dosage forms may also include, as is normal practice, additional substances other than inert diluents, e.g., lubricating agents such as magnesium stearate. In the case of capsules, tablets, and pills, the dosage forms may also include buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

Effective amounts of the compounds of the invention generally include any amount sufficient to detectably treat the disorders described herein.

Successful treatment of a subject in accordance with the invention may result in a reduction or alleviation of symptoms in a subject afflicted with a medical or biological disorder. For example, treatment may halt the further progression of the disorder, or may prevent or retard development of the disorder.

The amount of active ingredient that may be combined with the carrier materials to produce a single dosage form will vary depending upon the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and severity of the particular disease undergoing therapy. The therapeutically effective amount for a given situation can be readily determined by routine experimentation and is within the skill and judgment of the ordinary clinician.

### DEFINITIONS

As used above and elsewhere herein the following terms and abbreviations have the meanings defined below:
AcH Acetic Acid
ATP Adenosine triphosphate
BCG Mycobacterium bovis bacillus Calmette-Guerin
Bn Benzyl
BSA Bovine Serum Albumin
DCM Dichloromethane
DIEA N,N-diisopropyl-ethylamine
EDC 1-(3-Dimethylaminopropyl)-3-ethylcarbodiimide hydrochloride
FHA Filamentous haemaglutinin
GCMS Gas Chromatography/Mass Spectroscopy
H. Pylori Helicobacter Pylori
HAV Hepatitis A Virus
HBV Hepatitis B Virus
HBr Hydrogen Bromide
HCV Hepatitis C Virus
HIV Human Immunodeficiency Virus
HPLC High Performance Liquid Chromatography
HSV Herpes Simplex Virus
IC₅₀ value The concentration of an inhibitor that causes a 50% reduction in a measured activity.
IFN Interferon
IL Interleukin
IMS Immunomagnetic separation
IPV Inactivated polio virus
LCMS Liquid Chromatography/Mass Spectroscopy
LPS Lipid polysaccharide
MAb or mAb Monoclonal Antibody
Men A Neisseria Meningitidis Type A
Men C Neisseria Meningitidis Type C
Men B Neisseria Meningitidis Type B
Men W Neisseria Meningitidis Type W
Men Y Neisseria Meningitidis Type Y
MeOH Methanol
MW Molecular Weight
NANB Non-A, non-B hepatitis
NMR Nuclear magnetic resonance
OMV Outer membrane vesicle
PBMC Peripheral blood mononuclear cells
PT Pertussis holotoxin
Rt Room temperature (25° C.)
SMIP Small Molecule Immune Potentiator
tBOK Potassium Tertiary Butoxide
TEA Triethylamine
OTf Triflate
THF Tetrahydrofuran
TLC Thin Layer Chromatography and/or Tender Loving Care
TMS Trimethylsilyl
TNF-α Tumour necrosis factor-alpha

The term “SMIP” refers to a small molecule immunopotentiating compound, including small molecule compounds, generally below about MW 800 g/mol, capable of stimulating or modulating a pro-inflammatory response in a patient. In some embodiments, the SMIP compounds are able to stimulate human peripheral blood mononuclear cells to produce cytokines. More particularly, preferred SMIPs include imidazoquinolines and those compounds encompassed by Formula I described herein, or contained within any reference cited herein.

The term “SMIS” refers to a small molecule immunosuppressant compound, including small molecule compounds, generally below about MW 800 g/mol, capable of suppressing or modulating an immune response in a patient. In some embodiments, the SMIS compounds are able to inhibit human peripheral blood mononuclear cell's ability to produce cytokines, chemokines, and/or growth factors. In other embodiments, the SMIS compounds are able to induce TGF-beta production, thereby suppressing an immune response.

Reference to “imidazoquinolines” (as pertaining to imidazoquinolines of the present invention), indicates compounds having the general structure of formula I as described herein. In some embodiments, the imidazoquinolines are selected from one of those in the following list:
N2-methyl-1-(2-methylpropyl)-1H-imidazo[4,5-c]quinoline-2,4-diamine;
N2,N2-dimethyl-1-(2-methylpropyl)-1H-imidazo[4,5-c]quinoline-2,4-diamine;
N2-ethyl-N2-methyl-1-(2-methylpropyl)-1H-imidazo[4,5-c]quinoline-2,4-diamine;
N2-methyl-1-(2-methylpropyl)-N2-propyl-1H-imidazo[4,5-c]quinoline-2,4-diamine;
1-(2-methylpropyl)-N2-propyl-1H-imidazo[4,5-c]quinoline-2,4-diamine;
N2-butyl-1-(2-methylpropyl)-1H-imidazo[4,5-c]quinoline-2,4-diamine;
N2-butyl-N2-methyl-1-(2-methylpropyl)-1H-imidazo[4,5-c]quinoline-2,4-diamine;
N2-methyl-1-(2-methylpropyl)-N2-pentyl-1H-imidazo[4,5-c]quinoline-2,4-diamine;
N2-methyl-1-(2-methylpropyl)-N2-prop-2-enyl-1H-imidazo[4,5-c]quinoline-2,4-diamine;
1-(2-methylpropyl)-2-[(phenylmethyl)thio]-1H-imidazo[4,5-c]quinolin-4-amine;
1-(2-methylpropyl)-2-(propylthio)-1H-imidazo[4,5-c]quinolin-4-amine; 2-[[4-amino-1-(2-methylpropyl)-1H-imidazo[4,5-c]quinolin-2-yl](methyl)amino]ethanol;
2-[[4-amino-1-(2-methylpropyl)-1H-imidazo[4,5-c]quinolin-2-yl](methyl)amino]ethyl acetate;
4-amino-1-(2-methylpropyl)-1,3-dihydro-2H-imidazo[4,5-c]quinolin-2-one;
N2-butyl-1-(2-methylpropyl)-N4,N4-bis(phenylmethyl)-1H-imidazo[4,5-c]quinoline-2,4-diamine;
N2-butyl-N2-methyl-1-(2-methylpropyl)-N4,N4-bis(phenylmethyl)-1H-imidazo[4,5-c]quinoline-2,4-diamine;
N2-methyl-1-(2-methylpropyl)-N4,N4-bis(phenylmethyl)-1H-imidazo[4,5-c]quinoline-2,4-diamine;
N2,N2-dimethyl-1-(2-methylpropyl)-N4,N4-bis(phenylmethyl)-1H-imidazo[4,5-c]quinoline-2,4-diamine;
1-{4-amino-2-[methyl(propyl)amino]-1H-imidazo[4,5-c]quinolin-1-yl}-2-methylpropan-2-ol;
1-[4-amino-2-(propylamino)-1H-imidazo[4,5-c]quinolin-1-yl]-2-methylpropan-2-ol; or
N4,N4-dibenzyl-1-(2-methoxy-2-methylpropyl)-N2-propyl-1H-imidazo[4,5-c]quinoline-2,4-diamine.

The term “refractory cancer cells” refers to cancer cell lines that are resistant to preexisting therapeutics or treatment regimens, including prescribed dosing schedules.

The methods of the invention are useful in treating “allergic diseases,” which may be accomplished in the same manner as the other immunotherapeutic methods described herein.

An “allergen” refers to a substance (antigen) that can induce an allergic or asthmatic response in a susceptible subject. The list of allergens is enormous and can include pollens, insect venoms, animal dander, dust, fungal spores, and drugs (e.g., penicillin).

“Asthma” refers to a disorder of the respiratory system characterized by inflammation, narrowing of the airways, and increased reactivity of the airways to inhaled agents. Asthma is frequently, although not exclusively, associated with atopic or allergic symptoms.

The term “leukotriene inhibitor” includes any agent or compound that inhibits, restrains, retards, or otherwise interacts with the action or activity of leukotrienes, such as, but not limited to, 5-lipoxygenase (“5-LO”) inhibitors, 5-lipoxygenase activating protein (“FLAP”) antagonists, and leukotriene D4 (“LTD4”) antagonists.

“Modulating” refers to inducing or suppressing.

“Immune-stimulation” or “immune potentiation” refers to activation of the immune system, including humoral or cellular activation, for example, activation of a cell, such as a killer (T or NK) or dendritic cell of the immune system, for example, causing the increase in cytokine production from a dendritic cell leading to an overall enhancement of host defense (immune response).

“Modulating an immune response” refers to either immune potentiation or immune suppression as defined herein.

An “immunogenic composition” refers to a composition capable of stimulating an immune response. In some embodiments, “immunogenic compositions” are compositions capable of stimulating an immune response in a subject. In some embodiments, the immunogenic composition is capable of modulating the production of cytokines in a subject, thereby effecting immune potentiation in that subject.

“Immune suppression” or “immunosuppression” refers to deactivation of the immune system, for example, preventing or lessening cytokine production from a dendritic cell leading to an overall attenuation of host defense (immune response).

An “immune-stimulatory effective amount” is an amount effective for activating the immune system, for example, causing an increase in cytokine production from a dendritic cell leading to an overall enhancement of host defense (immune response).

“Enhancing the immune response to an antigen” by a compound refers to enhancement of the immune response in comparison to that in the absence of the compound. An enhanced immune-response eliciting composition is a composition generally comprising an antigen and a small molecule immune potentiator compound that elicits an immune response greater than a composition comprising an antigen and not containing one or more small molecule immune potentiator compounds. In such embodiments, the compound acts as an adjuvant, for example, for use in vaccine compositions and methods.

A “disease associated with cellular proliferation” includes, but is not limited to neuro-fibromatosis, atherosclerosis, pulmonary fibrosis, arthritis, psoriasis, glomerulonephritis, restenosis, proliferative diabetic retinopathy (PDR), hypertrophic scar formation, inflammatory bowel disease, transplantation rejection, angiogenesis, and endotoxic shock.

The term “effective amount” is an amount necessary or sufficient to realize a desired biological effect. For example, an effective amount of a compound to treat an infectious disorder may be an amount necessary to cause an antigen specific immune response upon exposure to an infectious agent. The effective amount may vary, depending, for example, upon the condition treated, weight of the subject and severity of the disease. One of skill in the art can readily determine the effective amount empirically without undue experimentation.

As used herein “an effective amount for treatment” refers to an amount sufficient to palliate, ameliorate, stabilize, reverse, slow or delay progression of a condition such as a disease state.

Reference to “metronomic administration” or “administered metronomically” refers to increasingly frequent dosing regimens, at lower drug concentrations, as compared with known dosing regimens for an existing therapeutic. Metronomic administration varies from the typical dosing of cytotoxic drugs, which involves episodic (less frequent) administration at maximum tolerated doses (MTDs).

A “subject” or “patient” is meant to describe a human or vertebrate animal including a dog, cat, pocket pet, marmoset, horse, cow, pig, sheep, goat, elephant, giraffe, chicken, lion, monkey, owl, rat, squirrel, slender loris, and mouse.

A “pocket pet” refers to a group of vertebrate animals capable of fitting into a commodious coat pocket such as, for example, hamsters, chinchillas, ferrets, rats, guinea pigs, gerbils, rabbits and sugar gliders. Further description is provided by Mackay, B., Pocket Pets, Animal Issues, 32(1) 2001.

As used herein, the term “pharmaceutically acceptable ester” refers to esters, which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Representative examples of particular esters include, but are not limited to, formates, acetates, propionates, butyrates, acrylates and ethylsuccinates.

The compounds of the present invention can be used in the form of salts as in “pharmaceutically acceptable salts” derived from inorganic or organic acids. These salts include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-napthalenesulfonate, oxalate, pamoate, pectinate, sulfate, 3-phenylpropionate, picrate, pivalate, propionate, succinate, tartrate, thiocyanate, p-toluenesulfonate and undecanoate. Also, the basic nitrogen-containing groups can be quaternized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oil-soluble or dispersible products are thereby obtained.

The term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987. Prodrugs as described in U.S. Pat. No. 6,284,772 for example may be used.

The symbol [symbol] is meant to indicate the point of attachment of an appendage.

Reference to “halo,” “halide,” or “halogen” refers to F, Cl, Br, or I atoms, especially F, Cl, and Br.

Reference to “activated” or “activating” as applied to an R group, such as R₁₅, implies having an electrophilic moiety bound to an R group, capable of being displaced by a nucleophile. Examples of preferred activating groups are halogens, such as C1, Br or I, and F; triflates; esters; aldehydes; ketones; epoxides; and the like. An example of an activated R group is iodopropane, which is readily attacked by a nucleophile, such as a thiol to form a thiopropane functionality.

The term “coupling agent” refers to an agent that acts as an interface between two substituents, optionally forming a chemical bridge between the two to facilitate completion of the reaction. One preferred coupling agent is EDC.

The term “deprotecting” refers to removal of a protecting group, such as removal of a benzyl group bound to an amine. Deprotecting may be preformed by heating and/or addition of reagents capable of removing protecting groups. One preferred method of removing benzyl groups from amino groups is to add HBr and acetic acid with heat. Many deprotecting reactions are well known in the art and are described in Protective Groups in Organic Synthesis, Greene, T. W., John Wiley & Sons, New York, N.Y., (1st Edition, 1981).

Reference to “optionally purifying” indicates optionally removing components of a mixture that are not the desired product. Those components may be side products, remaining starting materials, or molecules that were introduced to the mixture somewhere in the process, such as water. “Purifying” thus encompasses chromatography, distillation, recrystallization, and filtration, as well as extractions, and drying or azeotroping with materials such as sodium sulfate or toluene.

Reference to “oxidizing” indicates formation of a bond to an atom more electronegative than the atom. Addition of hydrogen to an organic molecule is almost always regarded as a reduction. Oxidation may be accomplished using a variety of oxidizing agents which are well known to those skilled in the art. Reduction may be accomplished using a wide variety of reducing agents which are also well-known in the art.

“Reacting” refers to modifying conditions in a vessel such that an unreactive molecule becomes reactive. This may involve addition of solvent(s), a catalyst, reagents, coupling agents, and/or heat, among others.

Reference to “Pearlman's Catalyst” indicates palladium hydroxide on activated charcoal.

The phrase “alkyl” refers to substituted and unsubstituted alkyl groups such as methyl, ethyl, propyl, butyl, pentyl, hexyl, heptyl, octyl, nonyl, decyl, undecyl, dodecyl and the like. The phrase “C₁₋₆ alkyl” has the same meaning as alkyl, except that it is limited to alkyl groups of six carbons or less. The phrase C₁₋₆ alkyl also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃)₃, —CH₂CH(CH₃)₂, —CH₂CH(CH₃)(CH₂CH₃), —CH₂CH(CH₂CH₃)₂, —CH₂C(CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂C(CH₃)₃, —CH(CH₃)CH₂CH(CH₃)₂, —CH(CH₃)CH(CH₃)CH(CH₃), and others. The phrase C₁₋₆ alkyl further includes cyclic alkyl or C₃₋₆cycloalkyl groups such as cyclopropyl, cyclobutyl, cyclopentyl, cyclohexyl, and such rings substituted with straight and branched chain alkyl groups as defined above. The phrase alkyl also includes polycyclic alkyl groups such as, but not limited to, adamantyl norbornyl, and bicyclo[2.2.2]octyl and such rings substituted with straight and branched chain alkyl groups as defined above.

The phrase “aryl” refers to substituted and unsubstituted aryl groups that do not contain heteroatoms. The phrase “C₆₋₁₀ aryl” has the same meaning as aryl, except that it is limited to aryl groups of six to ten carbons atoms. The phrase aryl includes, but is not limited to, groups such as phenyl, biphenyl, and naphthyl by way of example. Aryl groups also include those in which one of the aromatic carbons is bonded to an alkyl, alkenyl, or alkynyl group as defined herein. This includes bonding arrangements in which two carbon atoms of an aryl group are bonded to two atoms of an alkyl, alkenyl, or alkynyl group to define a fused ring system (e.g. dihydronaphthyl or tetrahydronaphthyl). Thus, the phrase “aryl” includes, but is not limited to tolyl, and hydroxyphenyl among others.

The phrase “alkenyl” refers to straight chain, branched chain, and cyclic groups such as those described with respect to alkyl groups as defined above, except that at least one double bond exists between two carbon atoms. The phrase “C₂₋₆ alkenyl” has the same meaning as alkenyl, except that it is limited to alkenyl groups of two to six carbons. Examples include, but are not limited to, vinyl, —CH═C(H)(CH₃), —CH═C(CH₃)₂, —C(CH₃)═C(H)₂, —C(CH₃)═C(H)(CH₃), —C(CH₂CH₃)═CH₂, cyclohexenyl, cyclopentenyl, cyclohexadienyl, butadienyl, pentadienyl, hexadienyl, and the like.

The phrase “alkoxy” refers to groups having the formula —O-alkyl, wherein the point of attachment is the oxy group and the alkyl group is as defined above. The phrase “C₁₋₆ alkoxy” has the same meaning as alkoxy, except that it is limited to alkoxy groups having from one to six carbon atoms.

The phrase “aryloxy” refers to groups having the formula —O-aryl, wherein the point of attachment is the oxy group and the aryl group is as defined above. The phrase “C₆₋₁₀ aryloxy” has the same meaning as aryloxy, except that it is limited to aryloxy groups of six to ten carbon atoms.

The phrase “C₁₋₆ alkoxy-C₁₋₆ alkyl” refers to ether groups with as many as 12 carbon atoms. One example of a C₁₋₆ alkoxy-C₁₋₆ alkyl group is —CH₂—O—CH₂CH₃.

The phrase “C₆₋₁₀ aryloxy-C₁₋₆ alkyl” refers to aryl ether groups of 16 carbon atoms or less, especially of 10 carbon atoms or less bound at the C₁₋₆ alkyl group. One example of a C₆₋₁₀ aryloxy-C₁₋₆ alkyl group is propoxybenzene.

The phrase “C₆₋₁₀ aryl-C₁₋₆ alkyl” refers to arylalkyl groups of 16 carbon atoms or less, especially of 10 carbon atoms or less bound at the C₁₋₆ alkyl group. One example of a C₆₋₁₀ aryl-C₁₋₆ alkyl group is toluene.

The phrase “alkynyl” refers to straight and branched chain groups such as those described with respect to alkyl groups as defined above, except that at least one triple bond exists between two carbon atoms. The phrase “C₂₋₆ alkynyl” has the same meaning as alkynyl, except that it is limited to alkynyl groups of two to six carbons. Examples include, but are not limited to, —C≡C(H), —C≡C(CH₃), —C≡C(CH₂CH₃), —C(H₂)C≡C(H), —C(H)₂C≡C(CH₃), —C(H)₂C≡C(CH₂CH₃), and the like.

The phrase “trihalomethyl” refers to a methyl group in which the three H atoms of the methyl group are substituted with three halogens which may be same or different. One example of such a group is a —CF₃ group in which all three H atoms of the methyl group are substituted with F atoms.

For clarification, —CH₂C(CH₃)₂(OH) refers to 2-methylpropan-2-ol or tertbutanol.

The phrase “heterocyclyl” refers to both aromatic and nonaromatic ring compounds including monocyclic, bicyclic, and polycyclic ring compounds such as, but not limited to, quinuclidyl, containing 3 or more ring members of which one or more is a heteroatom such as, but not limited to, N, O, and S. Examples of heterocyclyl groups include, but are not limited to: unsaturated 3 to 8 membered rings containing 1 to 4 nitrogen atoms such as, but not limited to pyrrolyl, pyrrolinyl, imidazolyl, pyrazolyl, pyridyl, dihydropyridyl, pyrimidyl, pyrazinyl, pyridazinyl, triazolyl (e.g. 4H-1,2,4-triazolyl, 1H-1,2,3-triazolyl, 2H-1,2,3-triazolyl etc.), tetrazolyl, (e.g. 1H-tetrazolyl, 2H tetrazolyl, etc.); saturated 3 to 8 membered rings containing 1 to 4 nitrogen atoms such as, but not limited to, pyrrolidinyl, imidazolidinyl, piperidinyl, piperazinyl; condensed unsaturated heterocyclic groups containing 1 to 4 nitrogen atoms such as, but not limited to, indolyl, isoindolyl, indolinyl, indolizinyl, benzimidazolyl, quinolyl, isoquinolyl, indazolyl, benzotriazolyl; unsaturated 3 to 8 membered rings containing 1 to 2 oxygen atoms such as, but not limited to furanyl; unsaturated 3 to 8 membered rings containing 1 to 2 oxygen atoms and 1 to 3 nitrogen atoms such as, but not limited to, oxazolyl, isoxazolyl, oxadiazolyl (e.g. 1,2,4-oxadiazolyl, 1,3,4-oxadiazolyl, 1,2,5-oxadiazolyl, etc.); saturated 3 to 8 membered rings containing 1 to 2 oxygen atoms and 1 to 3 nitrogen atoms such as, but not limited to, morpholinyl; unsaturated condensed heterocyclic groups containing 1 to 2 oxygen atoms and 1 to 3 nitrogen atoms, for example, benzoxazolyl, benzoxadiazolyl, benzoxazinyl (e.g. 2H-1,4-benzoxazinyl etc.); unsaturated 3 to 8 membered rings containing 1 to 3 sulfur atoms and 1 to 3 nitrogen atoms such as, but not limited to, thiazolyl, isothiazolyl, thiadiazolyl (e.g. 1,2,3-thiadiazolyl, 1,2,4-thiadiazolyl, 1,3,4-thiadiazolyl, 1,2,5-thiadiazolyl, etc.); saturated 3 to 8 membered rings containing 1 to 2 sulfur atoms and 1 to 3 nitrogen atoms such as, but not limited to, thiazolodinyl; saturated and unsaturated 3 to 8 membered rings containing 1 to 2 sulfur atoms such as, but not limited to, thienyl, dihydrodithiinyl, dihydrodithionyl, tetrahydrothiophene, tetrahydrothiopyran; unsaturated condensed heterocyclic rings containing 1 to 2 sulfur atoms and 1 to 3 nitrogen atoms such as, but not limited to, benzothiazolyl, benzothiadiazolyl, benzothiazinyl (e.g. 2H-1,4-benzothiazinyl, etc.), dihydrobenzothiazinyl (e.g. 2H-3,4-dihydrobenzothiazinyl, etc.), unsaturated condensed heterocyclic rings containing 1 to 2 oxygen atoms such as benzodioxolyl (e.g. 1,3-benzodioxoyl, etc.); unsaturated 3 to 8 membered rings containing an oxygen atom and 1 to 2 sulfur atoms such as, but not limited to, dihydrooxathiinyl; saturated 3 to 8 membered rings containing 1 to 2 oxygen atoms and 1 to 2 sulfur atoms such as 1,4-oxathiane; unsaturated condensed rings containing 1 to 2 sulfur atoms such as benzothienyl, benzodithiinyl; and unsaturated condensed heterocyclic rings containing an oxygen atom and 1 to 2 oxygen atoms such as benzoxathiinyl. Heterocyclyl group also include those described above in which one or more S atoms in the ring is double-bonded to one or two oxygen atoms (sulfoxides and sulfones). For example, heterocyclyl groups include tetrahydrothiophene, tetrahydrothiophene oxide, and tetrahydrothiophene 1,1-dioxide. Preferred heterocyclyl groups contain 5 or 6 ring members. More preferred heterocyclyl groups include morpholine, piperazine, piperidine, pyrrolidine, imidazole, pyrazole, 1,2,3-triazole, 1,2,4-triazole, tetrazole, thiomorpholine, thiomorpholine in which the S atom of the thiomorpholine is bonded to one or more O atoms, pyrrole, homopiperazine, oxazolidin-2-one, pyrrolidin-2-one, oxazole, quinuclidine, thiazole, isoxazole, furan, and tetrahydrofuran. “Heterocyclyl” also refers to those groups as defined above in which one of the ring members is bonded to a non-hydrogen atom such as described above with respect to substituted alkyl groups and substituted aryl groups. Examples, include, but are not limited to, 2-methylbenzimidazolyl, 5-methylbenzimidazolyl, 5-chlorobenzthiazolyl, 1-methyl piperazinyl, and 2-chloropyridyl among others. Heterocyclyl groups are those limited to having 2 to 15 carbon atoms and as many as 6 additional heteroatoms as described above. More preferred heterocyclyl groups have from 3 to 5 carbon atoms and as many as 2 heteroatoms. Most preferred heterocyclyl groups include piperidinyl, pyrrolidinyl, azetidinyl, and aziridinyl groups.

The term “substituted” refers to the replacement of one or more hydrogen atom with a monovalent or divalent radical. Suitable substitution groups include, for example, hydroxyl, nitro, amino, imino, cyano, halo, thio, thioamido, amidino, imidino, oxo, oxamidino, methoxamidino, imidino, guanidino, sulfonamido, carboxyl, formyl, alkyl, heterocyclyl, aryl, haloalkyl, alkoxy, alkoxyalkyl, alkylcarbonyl, arylcarbonyl, aralkylcarbonyl, alkylthio, aminoalkyl, alkylamino, cyanoalkyl, and the like. For example, one preferred “substituted C₁₋₆ alkyl” is tertbutanol. Another preferred substituted C₁₋₆ alkyl is —CH₂C(CH₃)₂NH—SO₂CH₃.

The substitution group can itself be substituted one time. For example, an alkoxy substituent of an alkyl group may be substituted with a halogen, and oxo group, an aryl group, or the like. The group substituted onto the substitution group can be carboxyl, halo, nitro, oxo, amino, cyano, hydroxyl, C₁₋₆ alkyl, C₁₋₆ alkoxy, C₆₋₁₀ aryl, aminocarbonyl, —SR, thioamido, —SO₃H, —SO₂R or cycloalkyl, where R is typically hydrogen, hydroxyl or C₁₋₆ alkyl.

When the substituted substituent includes a straight chain group, the substitution can occur either within the chain (e.g., 2-hydroxypropyl, 2-aminobutyl, and the like) or at the chain terminus (e.g., 2-hydroxyethyl, 3-cyanopropyl, and the like). Substituted substituents can be straight chain, branched or cyclic arrangements of covalently bonded carbon atoms or heteroatoms.

The term “protected” or a “protecting group” with respect to hydroxyl groups, amine groups, and sulfhydryl groups refers to forms of these functionalities which are protected from undesirable reaction with a protecting group known to those skilled in the art such as those set forth in Protective Groups in Organic Synthesis, Greene, T. W., John Wiley & Sons, New York, N.Y., (1st Edition, 1981) which can be added or removed using the procedures set forth therein. Examples of protected hydroxyl groups include, but are not limited to, silyl ethers such as those obtained by reaction of a hydroxyl group with a reagent such as, but not limited to, t-butyldimethyl-chlorosilane, trimethylchlorosilane, triisopropylchlorosilane, triethylchlorosilane; substituted methyl and ethyl ethers such as, but not limited to methoxymethyl ether, methylhiomethyl ether, benzyloxymethyl ether, t-butoxymethyl ether, 2-methoxyethoxymethyl ether, tetrahydropyranyl ethers, 1-ethoxyethyl ether, allyl ether, benzyl ether; esters such as, but not limited to, benzoylformate, formate, acetate, trichloroacetate, and trifluoracetate. Examples of protected amine groups include, but are not limited to, benzyl or dibenzyl, amides such as, formamide, acetamide, trifluoroacetamide, and benzamide; imides, such as phthalimide, and dithiosuccinimide; and others. In some embodiments, a protecting group for amines is a benzyl group. Examples of protected sulfhydryl groups include, but are not limited to, thioethers such as S-benzyl thioether, and S-4-picolyl thioether; substituted S-methyl derivatives such as hemithio, dithio and aminothio acetals; and others.

Imidazoquinoline compounds of formula I may exhibit the phenomenon of tautomerism, and the formula drawings within this specification can represent only one of the possible tautomeric forms. It is to be understood that the invention encompasses any tautomeric form which possesses immunomodulatory activity and is not to be limited merely to any one tautomeric form utilized within the formula drawings.

Imidazoquinolines of formula I also may exist in solvated as well as unsolvated forms such as, for example, hydrated forms. The invention encompasses both solvated and unsolvated forms which possess immunomodulatory activity.

The invention also includes isotopically-labeled imidazoquinoline compounds, that are structurally identical to those disclosed above, except that one or more atom is/are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, sulfur, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively. Compounds of the present invention, tautomers thereof, prodrugs thereof, and pharmaceutically acceptable salts of the compounds and of the prodrugs that contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, may afford certain therapeutic advantages resulting from greater metabolic stability, for example increased in vivo half-life or reduced dosage requirements and, hence, may be preferred in some circumstances. Isotopically labeled compounds of this invention and prodrugs thereof can generally be prepared by carrying out known or referenced procedures and by substituting a readily available isotopically labeled reagent for a non-isotopically labeled reagent.

The foregoing may be better understood by reference to the following Examples that are presented for illustration and not to limit the scope of the inventive concepts. The Example compounds and their analogs are easily synthesized by one skilled in the art from procedures described herein, as well as in patents or patent applications listed herein which are all hereby incorporated by reference in their entireties and for all purposes as if fully set forth herein.

### EXAMPLES

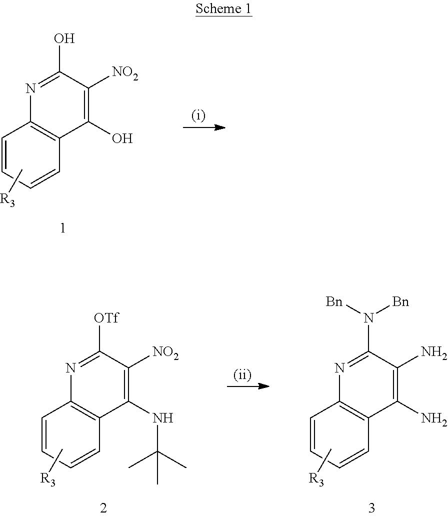

Reaction Scheme 1 illustrates preparation of a versatile intermediate for compounds of the invention. The scheme is further described in U.S. Pat. No. 548,293, which is incorporated herein by reference. The unsubstituted compound of Formula I is a known commercially available compound and other compounds of Formula I, including those substituted at R₃ as described herein, can be prepared by methods known to those skilled in the art and disclosed, e.g., in Chem. Ber. 1927, 60, 1108 (Kohler) and J. Heterocyclic Chem. 1988, 25, 857 (Kappe).

In step (i) a 3-nitroquinoline-2,4-disulfonate is first prepared by reacting a 2,4-dihydroxy-3-nitroquinoline with a sulfonyl halide or preferably a sulfonic anhydride. Suitable sulfonyl halides include alkylsulfonyl halides such as methanesulfonyl chloride and trifluoromethanesulfonyl chloride, and arylsulfonyl halides such as benzenesulfonyl chloride, p-bromobenzenesulfonyl chloride, and p-toluenesulfonyl chloride. Suitable sulfonic anhydrides include those corresponding to the above-mentioned sulfonyl halides. A particularly preferred sulfonic anhydride is trifluoromethanesulfonic anhydride.

Reaction conditions preferably involve first combining compound 1 with a base, preferably an excess of a tertiary amine base (e.g., a trialkylamine base such as triethylamine) and preferably in an appropriate solvent such as dichloromethane and then adding the sulfonyl halide or the sulfonic anhydride. The addition is preferably carried out in a controlled fashion (e.g., dropwise) and at a reduced temperature (e.g., at about 0° C.).

The disulfonate is then reacted with tert-butylamine, preferably in the presence of an excess of a tertiary amine base in a solvent such as dichloromethane to afford compound 2. The reaction can be carried out by adding the tertiary amine base to the reaction mixture resulting from the first portion of step (i), cooling to a reduced temperature (e.g., 0° C.) and adding the tert-butylamine in a controlled fashion (e.g., dropwise). The reaction can also be carried out by adding the tert-butylamine to a solution of the disulfonate and a tertiary amine base in a solvent such as dichloromethane. The reaction can be run at relatively low temperatures, e.g., about 0° C., in order to decrease the amount of undesired 2-aminated and 2,4-diaminated side products. It is sometimes necessary or desirable to heat the reaction mixture after the addition in order to complete the reaction.

In step (ii) the compound 2 is reacted with dibenzylamine. The reaction can be carried out by placing the starting material and the dibenzylamine in an inert solvent such as benzene, toluene, or xylene, and heating at a temperature and for a time sufficient to cause displacement of the sulfonate group by the dibenzylamine, such temperature and time being readily selected by those skilled in the art. The tert-butyl group is then removed by heating in a polar solvent: such as methanol in the presence of an acid such as hydrochloric acid.

The nitro group is then reduced to an amino group. Methods for such a reduction are well known to those skilled in the art: A preferred method involves in situ generation of Ni₂B from sodium borohydride and NiCl₂ in methanol to afford a reducing agent solution. The nitro compound is added to the reducing agent solution to effect reduction of the nitro group. The product is compound 3. Subsequent addition of HCl, in the form of a gas bubbled through methanol, or dissolving in aqueous HCl followed by lyophilization affords the useful HCl intermediate described in many of the following schemes.

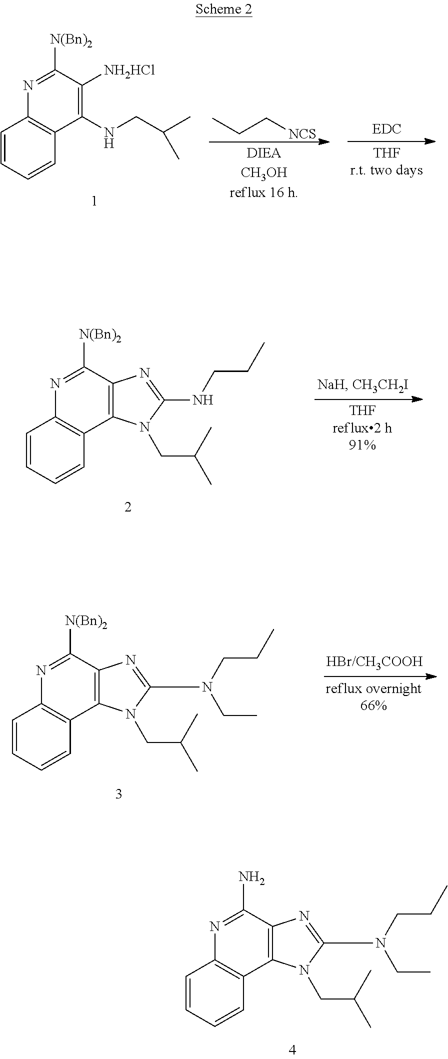

Step I

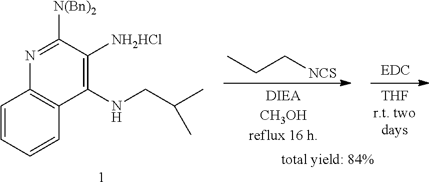

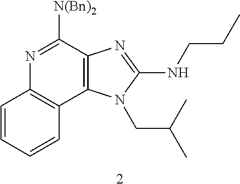

| | Sourced | | | | |
|---|---|---|---|---|---|
| Material | by: | MW | Qty. | Mole | Ration |
| 1 | | 447.01 | 2.235 g | 5 mmol | 1.0 eq. |
| N,N-diisopropyl- | IL | 129.24 | 0.96 mL | 5.5 mmol | 1.1 eq. |
| ethylamine(DIEA) | | | | | |
| methanol | LAB- | | 40 mL | | |
| | SCAN | | | | |
| propyl | Acros | 101.17 | 0.52 mL | 5 mmol | 1.0 eq. |
| isothiocyanate | | | | | |
| 1-(3-Dimethyl- | Acros | 191.7 | 1.438 mL | 7.5 mmol | 1.5 eq. |
| aminopropyl)3- | | | | | |
| ethylcarbodiimide | | | | | |
| hydrochloride | | | | | |
| (EDC) | | | | | |
| THF | LAB- | | 50 mL | | |
| | SCAN | | | | |

Compound 1 was synthesized as described in Scheme 1, using 2-methyl-1-propylamine instead of tert-butylamine in step (i) of the Scheme 1. Compound 1 (2.235 g, 5 mmol, 1.0 eq) was dissolved in dry methanol (40 mL), N,N-diisopropylethylamine (0.96 mL, 5.5 mmol, 1.1 eq.) was added. The solution was stirred for 0.5 hours, and then propyl isothiocyanate (0.52 mL, 5 mmol, 1.0 eq.) was added. After refluxing for 16 hours, the solution was concentrated and the residue was taken into THF (50 mL) and 1-(3-dimethylaminopropyl)-3-ethylcarbodiimide hydrochloride (EDC) (1.438 g, 7.5 mmol, 1.5 eq.) was added. The reaction solution was stirred at room temperature for two days. The mixture was concentrated and the residue was partitioned between ethyl acetate and water. The organic layer was washed with a saturated solution of sodium chloride, then dried and concentrated. The crude residue was chromatographed on a column of silica-gel. The column was eluted with a 10:1 mixture (v/v) hexane and ethyl acetate. Concentration of the combined fractions gave the product as yellow oil (2.0 g, 84%).

¹H NMR (300 MHz, CDCl₃) 0.87 (t, J=7.2 Hz, 3H), 1.04 (d, J=6.6 Hz, 6H), 1.57 (m, 2H), 2.38 (m, 1H), 3.32 (m, 2H), 3.86 (t, J=5.4 Hz, 1H), 3.96 (d, J=7.5 Hz, 2H), 5.38 (s, 4H), 7.17-7.81 (m, 14H); ¹³C NMR (300 MHz, CDCl₃) 12.2, 20.8, 23.6, 29.9, 46.3, 51.1, 52.4, 115.6, 119.1, 121.8, 126.1, 126.6, 127.2, 128.5, 128.9, 129.0, 133.8, 141.0, 144.6, 150.9, 152.2; HRMS (EI) obsd 477.2888 (M⁺), calcd 477.2889 (M⁺) (C₃₁H₃₅N₅).

Step II

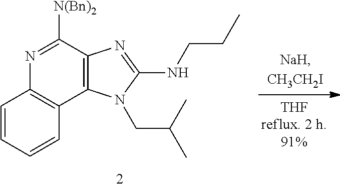

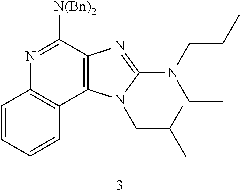

| | Sourced | | | | |
|---|---|---|---|---|---|
| Material | by: | MW | Qty. | Mole | Ration |
| 2 | prepared | 477.6 | 1.887 g | 3.95 mmol | 1.0 eq. |
| 60% sodium | Panreac | 24.0 | 0.316 g | 7.9 mmol | 2.0 eq. |
| hydride | | | | | |
| Iodoethane | Riedel-de | 156.0 | 0.48 mL | 5.93 mmol | 1.5 eq. |
| | Haën | | | | |
| THF | LAB-SCAN | | 30 mL | | |

To a solution of 2 (1.887 g, 3.95 mmol, 1.0 eq.) in THF (30 mL), was added 60% sodium hydride (0.316 g, 7.9 mmol, 2.0 eq.), followed by the addition of iodoethane (0.48 mL, 5.93 mmol, 1.5 eq.). The mixture was refluxed in an oil bath for 2 hours. The mixture was then concentrated and the residue was partitioned between ethyl acetate and water. The organic layer was washed with brine, and dried. Concentration gave an oil residue which was chromatographed on a column of silica-gel. The column was eluted with a 10:1 mixture (v/v) hexane and ethyl acetate. Concentration of the combined fractions gave the product as yellow solid (1.83 g, 92%).

mp 115.4-116.0° C.; ¹H NMR (300 MHz, CDCl₃) 0.798 (t, J=7.2 Hz, 3H), 0.80 (d, J=6.6 Hz, 6H), 1.01 (t, J=7.2 Hz, 3H), 1.45 (m, 2H), 2.30 (m, 1H), 3.00 (t, J=7.5 Hz, 2H), 3.10 (q, J=7.2 Hz, 2H), 4.16 (d, J=7.2 Hz, 2H), 5.38 (s, 4H), 7.17-7.81 (m, 14H); ¹³C NMR (300 MHz, CDCl₃) 11.7, 12.4, 19.7, 20.5, 28.3, 47.5, 50.4, 52.7, 54.5, 115.3, 119.5, 121.0, 126.0, 126.5, 127.6, 128.1, 128.2, 133.7, 140.1, 144.3, 150.5, 155.5; HRMS (EI) obsd 505.3198 (M⁺), calcd 505.3200 (M⁺) (C₃₃H₃₉N₅).

Step III

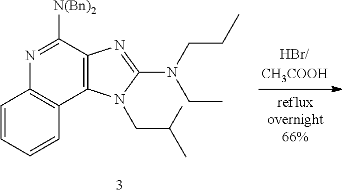

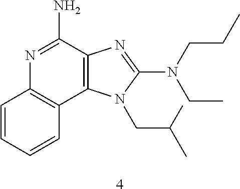 [SMILES: CCCN(CC)c1nc2c(N)nc3ccccc3c2n1CC(C)C ; "4"]

| | Sourced | | | | |
|---|---|---|---|---|---|
| Material | by: | MW | Qty. | Mole | Ration |
| 3 | prepared | 505.3 | 152 mg | 0.3 mmol | 1.0 eq. |
| Hydrogen bromide | Merck | | 10 mL | | |
| (47% in water) | | | | | |
| Acetic acid | Fisher | | 10 mL | | |

A solution of 3 (152 mg, 0.3 mmol, 1.0 eq.) in hydrogen bromide (10 mL, 47% in water) and acetic acid (10 mL) was refluxed overnight. Reaction solution was then diluted with CH₂Cl₂ (100 mL), and brought to pH 7 with a 1M NaOH solution and a saturated solution of NaHCO₃. The organic layer was separated, dried, and concentrated. The crude product was purified by chromatography using 5% methanol in CH₂Cl₂ to yield 66% of (4).

mp 139.2-142.8° C.; ¹H NMR (300 MHz, CDCl₃) 0.82 (d, J=6.6 Hz, 6H), 0.92 (t, J=7.2 Hz, 3H), 1.15 (t, J=7.2 Hz, 3H), 1.60 (m, 2H), 2.29 (m, 1H), 3.14 (t, J=7.5 Hz, 2H), 3.24 (q, J=7.2 Hz, 2H), 4.16 (d, J=7.2 Hz, 2H), 5.67 (s, 2H), 7.27-7.84 (m, 4H); ¹³C NMR (300 MHz, CDCl₃) 12.4, 13.4, 20.4, 21.4, 29.2, 48.1, 53.5, 55.1, 116.4, 120.6, 122.6, 126.2, 127.0, 127.6, 133.0, 144.7, 151.6, 158.1; HRMS (EI) obsd 325.2262 (M⁺), calcd 325.2261 (M⁺) (C₁₉H₂₇N₅).

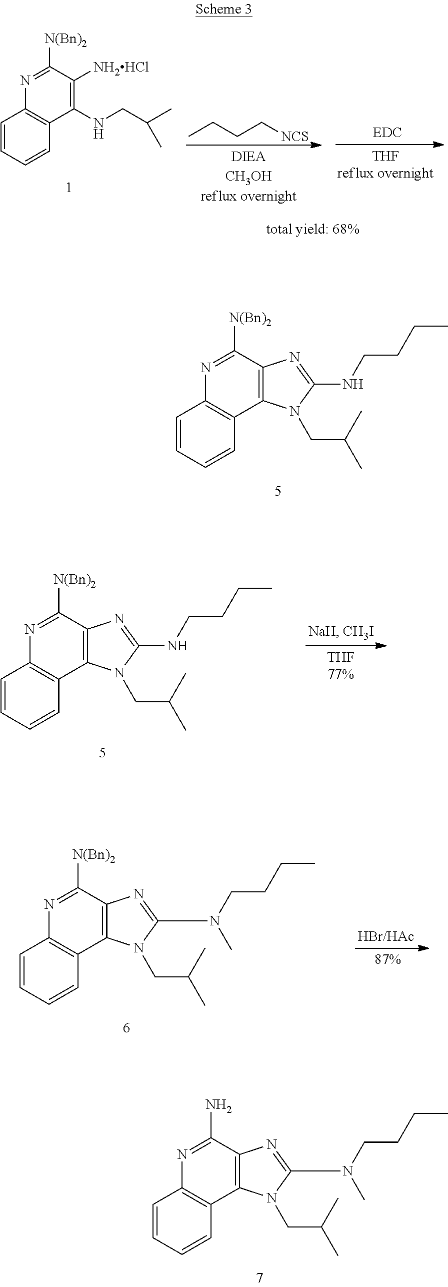

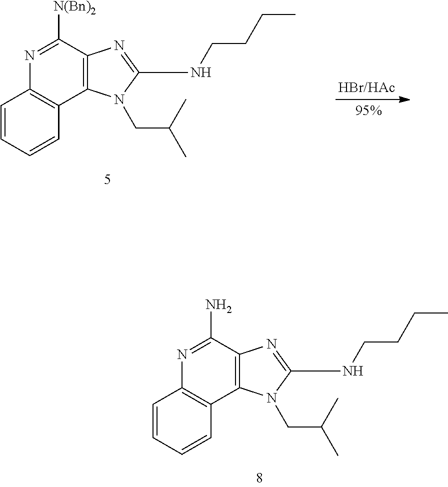

Step I

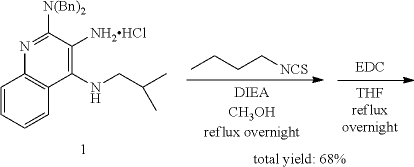

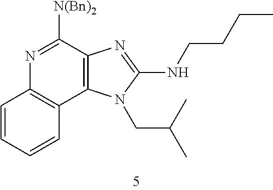

| | Sourced | | | | |
|---|---|---|---|---|---|
| Material | by: | MW | Qty. | Mole | Ration |
| 1 | | 447.01 | 536.4 mg | 1.2 mmol | 1.0 eq. |
| N,N-diisopropyl- | IL | 129.25 | 0.23 mL | 13.2 mmol | 1.1 eq. |
| ethylamine(DIEA) | | | | | |
| Methanol | LAB- | | 20 mL | | |
| | SCAN | | | | |
| n-butyl | Acros | 115.2 | 0.15 mL | 1.2 mmol | 1.0 eq. |
| isothiocyanate | | | | | |
| EDC | Acros | 191.7 | 460 mg | 2.4 mmol | 2.0 eq. |
| THF | LAB- | | 30 mL | | |
| | SCAN | | | | |

Compound 1 (536.4 mg, 1.2 mmol, 1.0 eq.) was dissolved in dry methanol (20 mL) and N,N-diisopropylethylamine (0.23 mL, 13.2 mmol, 1.1 eq.) was added. The solution was stirred for 0.5 hours, and then n-butyl isothiocyanate (0.15 mL, 1.2 mmol, 1.0 eq.) was added. After refluxing overnight, the solution was concentrated and taken up in THF (30 mL) and 1-(3-dimethylaminopropyl)-3-ethylcarbodiimide hydrochloride (EDC) (460 mg, 2.4 mmol, 2.0 eq.) was added. The reaction solution was refluxed overnight. The mixture was concentrated, and the residue was partitioned between ethyl acetate and water. The organic layer was washed with a saturated solution of sodium chloride, then dried and concentrated. The crude residue was chromatographed on a column of silica gel. The column was eluted with a 10:1 mixture (v/v) hexane and ethyl acetate. Concentration of the combined fractions gave the product as yellow oil (0.4 g, 68%).

¹H NMR (300 MHz, CDCl₃) 0.86 (t, J=7.2 Hz, 3H), 1.05 (d, J=6.6 Hz, 6H), 1.30 (m, 2H), 1.54 (m, 2H), 2.28 (m, 1H), 3.36 (m, 2H), 3.84 (t, J=5.4 Hz, 1H), 3.98 (d, J=7.5 Hz, 2H), 5.39 (s, 4H), 7.20-7.81 (m, 14H); ¹³C NMR (300 MHz, CDCl₃) 14.5, 20.8, 29.9, 32.6, 44.3, 51.2, 52.3, 115.5, 119.2, 121.9, 126.2, 126.6, 127.2, 128.2, 128.9, 129.0, 133.8, 140.8, 150.7, 152.3; HRMS (EI) obsd 491.3028 (M⁺), calcd 491.3043 (M⁺) (C₃₂H₃₇N₅).

Step II

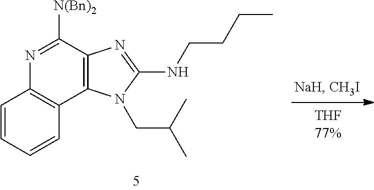

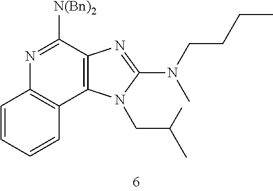

| | Sourced | | | | |
|---|---|---|---|---|---|
| Material | by: | MW | Qty. | Mole | Ration |
| 5 | Prepared | 491.3 | 208 mg | 0.42 mmol | 1.0 eq. |
| 60% sodium | Panreac | 24.0 | 50 mg | 1.26 mmol | 3.0 eq. |
| hydride | | | | | |
| iodomethane | Acros | 141.94 | 0.039 mL | 0.63 mmol | 1.5 eq. |
| THF | LAB- | | 30 mL | | |
| | SCAN | | | | |

To a solution of 5 (208 mg, 0.42 mmol, 1.0 eq.) in THF (30 mL) was added 60% sodium hydride (50 mg, 1.26 mmol, 3.0 eq.) which was followed by the addition of iodomethane (0.039 mL, 0.63 mmol, 1.5 eq.). The mixture was refluxed overnight under N₂. The mixture was concentrated, and the residue was partitioned between ethyl acetate and water. The organic layer was washed with brine, and dried with sodium sulfate. Concentration gave an oil residue which was chromatographed on a column of silica-gel. The column was eluted with a 10:1 mixture (v/v) hexane and ethyl acetate. Concentration of the combined fractions gave the product as oil (165 mg, 77%).

¹H NMR (300 MHz, CDCl₃) 0.80 (d, J=6.6 Hz, 6H), 0.84 (t, J=7.2 Hz, 3H), 1.25 (m, 2H), 1.47 (m, 2H), 2.29 (m, 1H), 3.01 (s, 3H), 3.03 (t, J=7.5 Hz, 2H), 4.17 (d, J=7.5 Hz, 2H), 5.38 (s, 4H), 7.20-7.85 (m, 14H); ¹³C NMR (300 MHz, CDCl₃) 14.7, 20.4, 21.1, 29.1, 30.1, 41.7, 51.1, 53.6, 56.1, 116.1, 120.2, 121.9, 126.9, 127.3, 128.4, 128.9, 129.1, 134.6, 140.9, 145.1, 151.3, 157.6; HRMS (EI) obsd 505.3187 (M⁺), calcd 505.3200 (M⁺) (C₃₃H₃₉N₅).

Step III

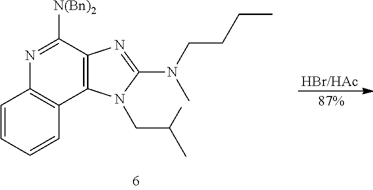

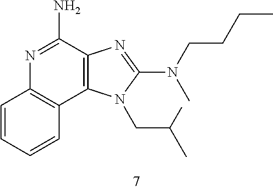 [SMILES: CCCCN(C)c1nc2c(N)nc3ccccc3c2n1CC(C)C]

| | Sourced | | | | |
|---|---|---|---|---|---|
| Material | by: | MW | Qty. | Mole | Ration |
| 6 | prepared | 505.3 | 140 mg | 0.28 mmol | 1.0 eq. |
| Hydrogen bromide | Merck | | 10 mL | | |
| (47% in water) | | | | | |
| Acetic acid | Fisher | | 10 mL | | |

A solution of 6 (140 mg, 0.28 mmol, 1.0 eq.) in hydrogen bromide (10 mL, 47% in water) and acetic acid (10 mL) was refluxed overnight. Reaction solution was diluted with CH₂Cl₂(100 mL) and brought to pH 7 with a 1M NaOH solution and a saturated solution of NaHCO₃. The organic layer was separated, dried, and concentrated. The crude product was purified by chromatography using 5% methanol in dichloromethane to afford a yield of 87% of (7).

mp 112.7-114.8° C.; ¹H NMR (300 MHz, CDCl₃) 0.82 (d, J=6.6 Hz, 6H), 0.95 (t, J=7.5 Hz, 3H), 1.39 (m, 2H), 1.62 (m, 2H), 2.26 (m, 1H), 2.89 (s, 3H), 3.16 (t, J=7.8 Hz, 2H), 4.18 (d, J=7.5 Hz, 2H), 5.50 (s, 2H), 7.26-7.85 (m, 4H); ¹³C NMR (300 MHz, CDCl₃) 14.0, 19.6, 20.2, 28.5, 29.5, 40.5, 52.9, 55.4, 115.7, 119.8, 122.0, 125.3, 126.4, 126.9, 132.4, 143.9, 150.7, 158.8; HRMS (EI) obsd 325.2262 (M⁺), calcd 325.2261 (M⁺) (C₁₉H₂₇N₅)

Step IV (to produce unmethylated analog):

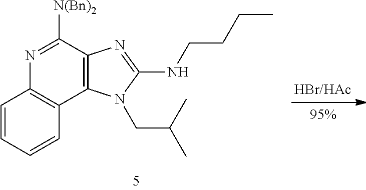

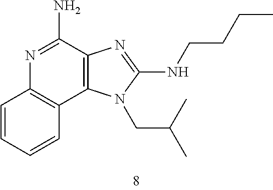 [SMILES: CCCCNc1nc2c(N)nc3ccccc3c2n1CC(C)C]

| | Sourced | | | | |
|---|---|---|---|---|---|
| Material | by: | MW | Qty. | Mole | Ration |
| 5 | prepared | 491.3 | 100 mg | 0.20 mmol | 1.0 eq. |
| Hydrogen | Merck | | 10 mL | | |
| bromide | | | | | |
| (47% in water) | | | | | |
| Acetic acid | Fisher | | 10 mL | | |

A solution of 5 (100 mg, 0.20 mmol, 1.0 eq.) in hydrogen bromide (10 mL, 47% in water) and acetic acid (10 mL) was refluxed overnight. The reaction solution was diluted with CH₂Cl₂ (100 mL), and brought to pH 7 with a 1M NaOH solution and a saturated solution of NaHCO₃. The organic layer was separated, dried, and concentrated. The crude product was purified by chromatography using 10% methanol in dichloromethane to provide (8) in a 95% yield.

¹H NMR (300 MHz, CDCl₃) 0.99 (t, J=7.3 Hz, 3H), 1.03 (d, J=6.9 Hz, 6H), 1.46 (m, 2H), 1.71 (m, 2H), 2.27 (m, 1H), 3.50 (m, 2H), 3.99 (d, J=7.8 Hz, 2H), 4.47 (t, 1H), 6.70 (s, 2H), 7.25-7.68 (m, 4H); ¹³C NMR (300 MHz, CDCl₃) 13.8, 19.8, 20.1, 29.0, 31.6, 43.4, 51.6, 113.6, 119.2, 122.9, 123.1, 124.0, 126.7, 132.3, 137.9, 148.7, 154.2; HRMS (EI) obsd 311.2106 (M⁺), calcd 325.2104 (M⁺) (C₁₈H₂₅N₅)

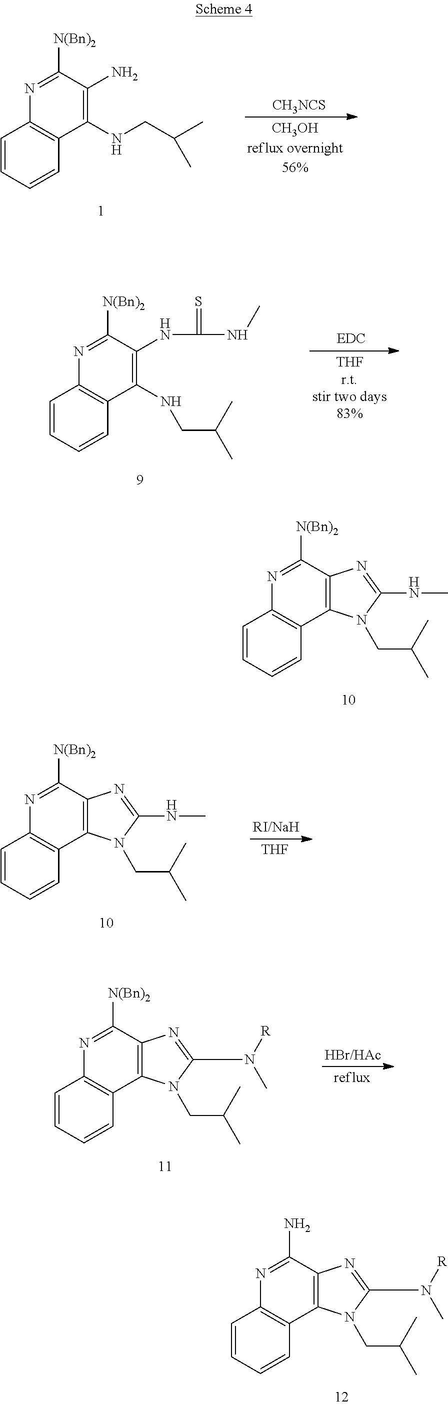

Step I

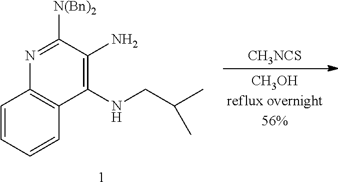

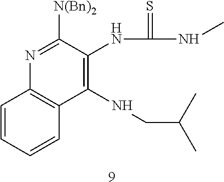

| | Sourced | | | | |
|---|---|---|---|---|---|
| Material | by: | MW | Qty. | Mole | Ration |
| 1 | prepared | 410.6 | 2.23 g | 5.4 mmol | 1.0 eq. |
| Methyl | Aldrich | 73.0 | 0.40 g | 5.4 mmol | 1.0 eq. |
| isothiocyanate | | | | | |
| methanol | LAB- | | 60 mL | | |
| | SCAN | | | | |

Compound 1 (2.23 g, 5.4 mmol, 1.0 eq.) was dissolved in dry methanol (60 mL), and methyl isothiocyanate (0.4 g, 5.4 mmol, 1.0 eq.) was added. After refluxing overnight, the solution was concentrated, and the residue was chromatographed on a column of silica-gel. The column was eluted with a 10:1 mixture (v/v) hexane and ethyl acetate. Concentration of the combined fractions gave the product 9 (1.46 g, 56%).

¹H NMR (300 MHz, CDCl₃) 0.88 (d, J=6.6 Hz, 6H), 1.60 (m, 1H), 2.97 (d, J=4.5 Hz, 3H), 3.24 (m, 2H), 4.73 (s, 5H), 5.54 (q, 1H), 6.92 (t, 1H), 7.17-7.78 (m, 14H); HRMS (EI) obsd 483.2455 (M⁺), calcd 483.2451 (M⁺) (C₂₉H₃₃N₅S₁)

Step II

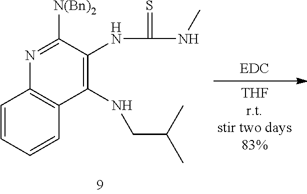

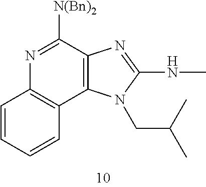

| Material | Sourced by: | MW | Qty. | | Mole | Ration |
|---|---|---|---|---|---|---|
| 9 | prepared | 483.2 | 416 | mg | 0.86 mmol | 1.0 eq. |
| 1-(3-Dimethyl- | Acros | 191.7 | 249 | mg | 1.3 mmol | 1.5 eq. |
| aminopropyl)3- | | | | | | |
| ethylcarbodiimide | | | | | | |
| hydrochloride | | | | | | |
| (EDC) | | | | | | |
| THF | LAB-SCAN | | 30 | mL | | |

Compound 9 (416 mg, 0.86 mmol, 1.0 eq.) was dissolved in THF (30 mL) and 1-(3-dimethylaminopropyl)-3-ethylcarbodiimide hydrochloride (EDC) (249 mg, 1.3 mmol, 1.5 eq.) was added. The reaction solution was stirred at room temperature for two days. The mixture was concentrated, and the residue was partitioned between ethyl acetate and water. The organic layer was washed with a saturated solution of sodium chloride, dried, and concentrated. The crude residue was chromatographed on a column of silica-gel. The column was eluted with a 10:1 mixture (v/v) hexane and ethyl acetate. Concentration of the combined fractions gave the product as white solid (320 mg, 83%).

mp 178.5-179.4° C.; ¹H NMR (300 MHz, CDCl₃) 1.04 (d, J=6.6 Hz, 6H), 2.39 (m, 1H), 2.99 (d, J=4.8 Hz, 3H), 3.83 (q, J=5.1 Hz, 1H), 3.98 (d, J=7.5 Hz, 2H), 5.40 (s, 4H), 7.18-7.82 (m, 14H); ¹³C NMR (300 MHz, CDCl₃) 20.7, 29.7, 31.2, 50.9, 52.2, 115.4, 119.0, 121.7, 126.1, 126.5, 127.1, 128.3, 128.8, 128.9, 133.9, 140.8, 144.5, 150.8, 152.8; HRMS (EI) obsd 449.2575 (M⁺), calcd 449.2574 (M⁺) (C₂₉H₃₁N₅)

Step III

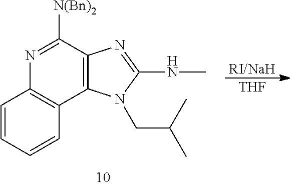

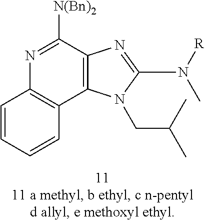

| | | Source* | MW of | Reaction | Reaction | |
|---|---|---|---|---|---|---|
| No. | R | of RI | RI* | condition | time | yield |
| 11a | methyl | Acros | 141.9 | Stir at r.t. | One day | 95% |
| 11b | ethyl | Riedel-de | 155.9 | Stir at r.t. | Two days | 96% |
| | | Haën | | | | |
| 11c | n-pentyl | Merck | 198.0 | Stir at r.t. | Two days | 54% |
| 11d | allyl | Aldrich | 167.98 | Stir at r.t. | overnight | 86% |
| 11e | Methoxyl | Aldrich | 139.0 | reflux | 8 h. | 76% |
| | ethyl | | | | | |

*In the case of 11e, 2-bromoethyl methyl ether was used as the reactant.

To a solution of 10 (135 mg, 0.3 mmol, 1.0 eq.) in THF (30 mL), was added 60% sodium hydride (36 mg, 0.9 mmol, 3.0 eq.), followed by the addition of an alkyl iodide (0.45 mmol, 1.5 eq.). The mixture was stirred at room temperature (or refluxed for 8 hours in the case of 11e). The mixture was concentrated, and the residue was partitioned between ethyl acetate and water. The organic layer was washed with brine, and dried with sodium sulfate. Concentration gave an oil residue which was chromatographed on a column of silica-gel. Concentration of the combined fractions gave the product as oil.

11a

¹H NMR (300 MHz, CDCl₃) 0.79 (d, J=6.6 Hz, 6H), 2.28 (m, 1H), 2.80 (s, 6H), 4.19 (d, J=7.5 Hz, 2H), 5.38 (s, 4H), 7.21-7.85 (m, 14H); ¹³C NMR (300 MHz, CDCl₃) 20.1, 28.9, 43.8, 50.9, 53.6, 115.9, 120.0, 121.8, 126.7, 127.1, 128.2, 128.7, 128.9, 140.7, 145.0; HRMS (EI) obsd 463.2726 (M⁺), calcd 463.2730 (M⁺) (C₃₀H₃₃N₅)

11b

¹H NMR (300 MHz, CDCl₃) 0.80 (d, J=6.6 Hz, 6H), 1.08 (t, J=7.2 Hz, 3H), 2.28 (m, 1H), 2.76 (s, 3H), 3.10 (q, J=7.2 Hz, 2H), 4.18 (d, J=7.5 Hz, 2H), 5.38 (s, 4H), 7.20-7.85 (m, 14H);

¹³C NMR (300 MHz, CDCl₃) 13.1, 20.2, 29.0, 41.0, 50.5, 51.0, 53.4, 115.9, 120.0, 121.7, 126.7, 127.1, 128.2, 128.8, 128.9, 134.4, 140.7, 145.0, 151.2, 157.1; HRMS (EI) obsd 477.2882 (M⁺), calcd 477.2887 (M⁺) (C₃₁H₃₅N₅)

11c

¹H NMR (300 MHz, CDCl₃) 0.80 (d, J=6.6 Hz, 6H), 0.85 (t, J=7.2 Hz, 3H), 1.23 (m, 4H), 1.49 (m, 2H), 2.28 (m, 1H), 2.77 (s, 3H), 3.02 (t, J=7.5 Hz, 2H), 4.17 (d, J=7.5 Hz, 2H), 5.38 (s, 4H), 7.20-7.82 (m, 14H); ¹³C NMR (300 MHz, CDCl₃) 14.6, 20.2, 23.1, 27.5, 28.9, 29.9, 41.5, 51.0, 53.4, 56.2, 116.0, 120.0, 121.7, 126.7, 127.1, 128.2, 128.7, 128.9, 134.4, 140.7, 145.0, 151.1, 157.5; HRMS (EI) obsd 519.3353 (M⁺), calcd 519.3356 (M⁺) (C₃₄H₄₁N₅)

11d

¹H NMR (300 MHz, CDCl₃) 0.80 (d, J=6.6 Hz, 6H), 2.25 (m, 1H), 2.76 (s, 3H), 3.64 (d, J=6.0 Hz, 2H), 4.18 (d, J=7.2 Hz, 2H), 5.15 (dd, J=10.2, 1.5 Hz, 1H), 5.25 (dd, J=17.1, 1.5 Hz, 1H), 5.38 (s, 4H), 5.83 (m, 1H), 7.21-7.82 (m, 14H); ¹³C NMR (300 MHz, CDCl₃) 20.2, 29.1, 40.7, 51.0, 53.4, 59.0, 116.0, 118.4, 120.0, 121.8, 126.7, 126.8, 127.2, 128.3, 128.8, 128.9, 134.5, 134.7, 140.7, 145.1, 151.2, 157.2; HRMS (EI) obsd 489.2886 (M⁺), calcd 489.2887 (M⁺) (C₃₂H₃₅N₅)

11e

¹H NMR (300 MHz, CDCl₃) 0.81 (d, J=6.6 Hz, 6H), 2.27 (m, 1H), 2.86 (s, 3H), 3.23 (s, 3H), 3.28 (t, J=5.4 Hz, 2H), 3.41 (t, J=5.4 Hz, 2H), 4.26 (d, J=7.2 Hz, 2H), 5.40 (s, 4H), 7.20-7.88 (m, 14H); ¹³C NMR (300 MHz, CDCl₃) 20.1, 28.9, 42.6, 51.2, 53.3, 55.5, 59.3, 70.6, 115.9, 120.1, 121.9, 126.5, 126.9, 127.2, 128.1, 128.8, 140.6, 145.0, 151.0, 157.0; HRMS (EI) obsd 507.3000 (M⁺), calcd 507.2993 (M⁺) (C₃₂H₃₇N₅O₁)

Step IV

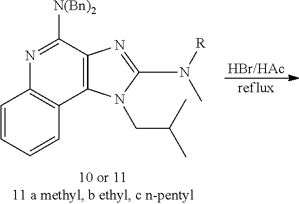

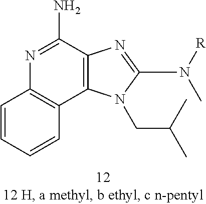

| No. | R | Reaction time | yield |
|---|---|---|---|
| 12 | H | overnight | 66% |
| 12a | methyl | Two days | 80% |
| 12b | ethyl | overnight | 52% |
| 12c | n-pentyl | overnight | 59% |

A solution of 10 or 11 (0.20 mmol, 1.0 eq.) in hydrogen bromide (10 mL, 47% in water) and acetic acid (10 mL) was refluxed overnight (or 2 days in the case of 12a). The reaction solution was diluted with CH₂Cl₂ (100 mL), and brought to pH 7 with a 1M NaOH solution and a saturated solution of NaHCO₃. The organic layer was separated, dried, and concentrated. The crude product was purified by chromatography using 5% methanol in dichloromethane.

12

decomposed 188.6° C.; ¹H NMR (300 MHz, CDCl₃) 1.01 (d, J=6.6 Hz, 6H), 2.28 (m, 1H), 3.13 (d, J=4.8 Hz, 3H), 3.97 (d, J=7.5 Hz, 2H), 4.51 (q, J=3.9 Hz, 1H), 6.70 (s, 2H), 7.25-7.86 (m, 4H); HRMS (EI) obsd 269.1637 (M⁺), calcd 269.1635 (M⁺) (C₁₅H₁₉N₅)

12a

decomposed 154.6° C.; ¹H NMR (300 MHz, CDCl₃) 0.82 (d, J=6.6 Hz, 6H), 2.18 (m, 1H), 2.93 (s, 6H), 4.19 (d, J=7.5 Hz, 2H), 7.28-7.94 (m, 4H); ¹³C NMR (300 MHz, CDCl₃) 20.0, 24.5, 29.2, 43.6, 53.9, 114.1, 120.8, 121.7, 124.4, 124.8, 128.8, 134.2, 137.2, 150.7, 160.7; HRMS (EI) obsd 283.1791 (M⁺), calcd 283.1791 (M⁺) (C₁₆H₂₁N₅)

12b

mp 183.6-186.8° C.; ¹H NMR (300 MHz, CDCl₃) 0.83 (d, J=6.6 Hz, 6H), 1.22 (t, J=7.2 Hz, 3H), 2.26 (m, 1H), 2.89 (s, 3H), 3.22 (q, J=7.2 Hz, 2H), 4.18 (d, J=7.2 Hz, 2H), 5.62 (s, 2H), 7.29-7.85 (m, 4H); ¹³C NMR (300 MHz, CDCl₃) 13.3, 20.2, 29.2, 40.6, 50.6, 53.6, 116.1, 120.5, 122.9, 127.0, 127.3, 133.2, 143.8, 151.1, 159.1; HRMS (EI) obsd 297.1945 (M⁺), calcd 297.1948 (M⁺) (C₁₇H₂₃N₅)

12c

mp 97.6-103.0° C.; ¹H NMR (300 MHz, CDCl₃) 0.80 (d, J=6.6 Hz, 6H), 0.90 (t, J=6.9 Hz, 3H), 1.32 (m, 4H), 1.61 (m, 2H), 2.24 (m, 1H), 2.87 (s, 3H), 3.13 (t, J=7.5 Hz, 2H), 4.16 (d, J=7.2 Hz, 2H); 5.64 (s, 2H), 7.25-7.83 (m, 4H); ¹³C NMR (300 MHz, CDCl₃) 14.6, 20.2, 23.1, 27.6, 29.1, 29.8, 41.0, 53.5, 56.3, 116.1, 120.5, 122.8, 125.8, 127.0, 127.2, 133.1, 143.9, 151.1, 159.5; HRMS (EI) obsd 339.2417 (M⁺), calcd 339.2417 (M⁺) (C₂₀H₂₉N₅)

Step V

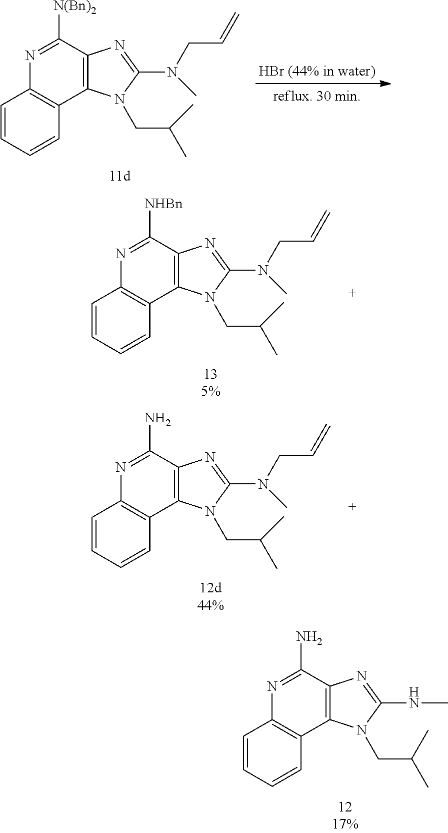

A solution of 11d (108 mg, 0.22 mmol, 1.0 eq.) in hydrogen bromide (10 mL, 47% in water) was refluxed for 0.5 hours. The reaction solution was diluted with CH₂Cl₂ (100 mL), and brought to pH 7 with a 1M NaOH solution and a saturated solution of NaHCO₃. The organic layer was separated, dried, and concentrated. Purification using chromatography (2.5%, 5%, and 20% methanol in dichloromethane sequentially) gave products 13 (5%), 12d (44%) and 12 (17%), respectively.

13

¹H NMR (300 MHz, CDCl₃) 0.82 (d, J=6.6 Hz, 6H), 2.62 (m, 1H), 2.84 (s, 3H), 3.71 (d, J=6.0 Hz, 2H), 4.15 (d, J=7.5 Hz, 2H), 4.95 (d, J=5.7 Hz, 2H), 5.25 (dd, J=10.2, 1.2 Hz, 1H), 5.38 (dd, J=17.1, 1.5 Hz, 1H), 5.94 (m, 1H), 6.99-7.87 (m, 9H); HRMS (EI) obsd 399.2419 (M⁺), calcd 399.2417 (M⁺) (C₂₅H₂₉N₅)

12d

¹H NMR (300 MHz, CDCl₃) 0.83 (d, J=6.6 Hz, 6H), 2.24 (m, 1H), 2.89 (s, 3H), 3.76 (d, J=6.0 Hz, 2H), 4.18 (d, J=7.5 Hz, 2H), 5.28 (dd, J=10.2, 1.5 Hz, 1H), 5.38 (dd, J=17.1, 1.5 Hz, 1H), 5.95 (m, 1H), 7.30-7.85 (m, 4H); HRMS (EI) obsd 309.1946 (M⁺), calcd 309.1948 (M⁺) (C₁₈H₂₃N₅)

Step VI

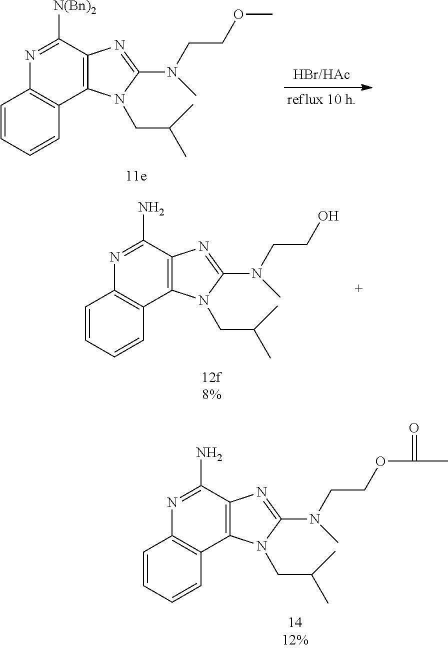

A solution of 11e (508 mg, 1 mmol, 1.0 eq.) in hydrogen bromide (15 mL, 47% in water) and acetic acid (15 mL) was refluxed for 10 hours. The reaction solution was diluted with CH₂Cl₂(100 mL) and brought to pH 7 with a 1M NaOH solution and a saturated solution of NaHCO₃. The organic layer was separated, dried, and concentrated. The crude product was purified by chromatography (4%, and 8% methanol in CH₂Cl₂ sequentially) giving products 12f (8%) and 14 (12%), respectively.

12f

¹H NMR (300 MHz, CDCl₃) 0.83 (d, J=6.3 Hz, 6H), 2.22 (m, 1H), 3.01 (s, 3H), 3.47 (t, J=4.8 Hz, 2H), 3.94 (t, J=4.8 Hz, 2H), 4.24 (d, J=7.5 Hz, 2H), 6.54 (s, 2H), 7.27-7.85 (m, 4H); HRMS (EI) obsd 313.1893 (M⁺), calcd 313.1897 (M⁺) (C₁₇H₂₃N₅O₁)

14

¹H NMR (300 MHz, CDCl₃) 0.83 (d, J=6.6 Hz, 6H), 2.02 (s, 3H), 2.29 (m, 1H), 2.96 (s, 3H), 3.59 (t, J=5.7 Hz, 2H), 4.22 (d, J=7.5 Hz, 2H), 4.33 (t, J=5.7 Hz, 2H), 5.43 (s, 2H), 7.31-7.85 (m, 4H); HRMS (EI) obsd 355.2006 (M⁺), calcd 355.2003 (M⁺) (C₁₉H₂₅N₅O₂)

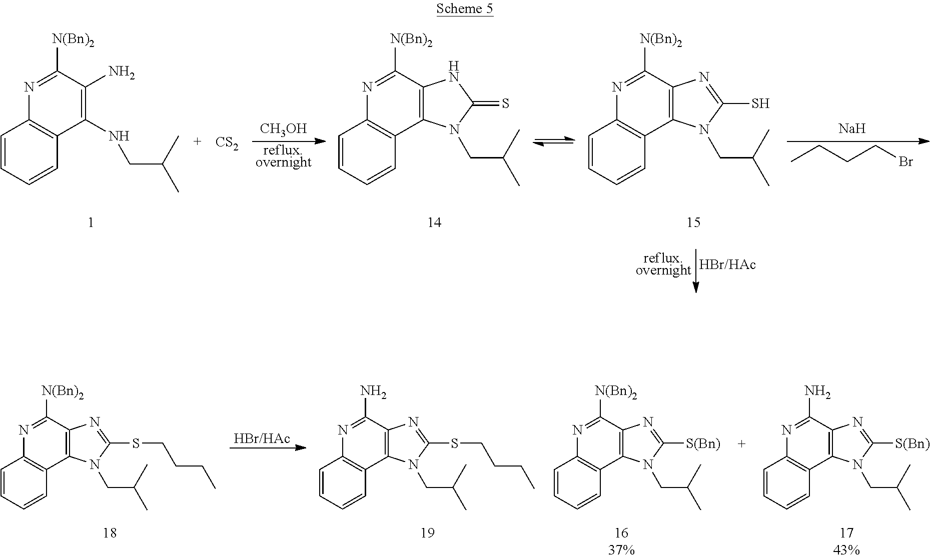

| Material | Sourced by: | MW | Qty. | | Mole | Ration |
|---|---|---|---|---|---|---|
| 1 | prepared | 410.6 | 205 | mg | 0.5 mmol | 1.0 eq. |
| CS₂ | Merck | 76.1 | 0.03 | mL | 0.5 mmol | 1.0 eq. |
| CH₃OH | LAB-SCAN | | 20 | mL | | |
| Hydrogen bromide | Merck | | 10 | mL | | |
| (47% in water) | | | | | | |
| Acetic acid | Fisher | | 10 | mL | | |

Step I

Compound 1 (205 mg, 0.5 mmol, 1.0 eq.) was dissolved in dry methanol (20 mL), followed by addition of carbon disulfide (0.03 mL, 0.5 mmol, 1.0 eq.). After refluxing overnight, the solution was concentrated and the residue was taken up in acetic acid (10 mL) and hydrogen bromide (10 mL, 47% in water). The reaction solution was then refluxed overnight. The mixture was diluted with CH₂Cl₂ (100 mL) and brought to pH 7 with a 1M NaOH solution and a saturated solution of NaHCO₃. The organic layer was separated, dried, and concentrated. The crude product was purified by chromatography using 2.5% and 5% methanol in dichloromethane sequentially, giving products 16 (37%) and 17(43%), respectively.

16

¹H NMR (300 MHz, CDCl₃) 0.93 (d, J=6.6 Hz, 6H), 2.27 (m, 1H), 4.12 (d, J=7.5 Hz, 2H), 4.48 (s, 2H), 4.97 (d, J=5.7 Hz, 2H), 6.07 (t, J=5.4 Hz, 1H), 7.23-7.82 (m, 14H); ¹³C NMR (300 MHz, CDCl₃) 20.4, 29.8, 39.2, 45.4, 53.8, 115.7, 120.3, 122.4, 127.5, 127.8, 128.3, 128.4, 128.8, 129.0, 129.2, 129.3, 129.6, 134.4, 137.4, 140.4, 145.8, 149.5, 150.5; MS (EI) 452 (M⁺).

17

¹H NMR (300 MHz, CDCl₃) 0.94 (d, J=6.9 Hz, 6H), 2.30 (m, 1H), 4.14 (d, J=7.8 Hz, 2H), 4.54 (s, 2H), 5.69 (s, 2H), 7.26-7.85 (m, 9H); ¹³C NMR (300 MHz, CDCl₃) 20.4, 29.7, 39.0, 53.8, 115.7, 120.4, 122.9, 127.6, 127.7, 128.3, 128.6, 129.3, 129.6, 135.1, 137.3, 145.0, 150.2, 151.2; MS (EI) 362 (M⁺).

Step II

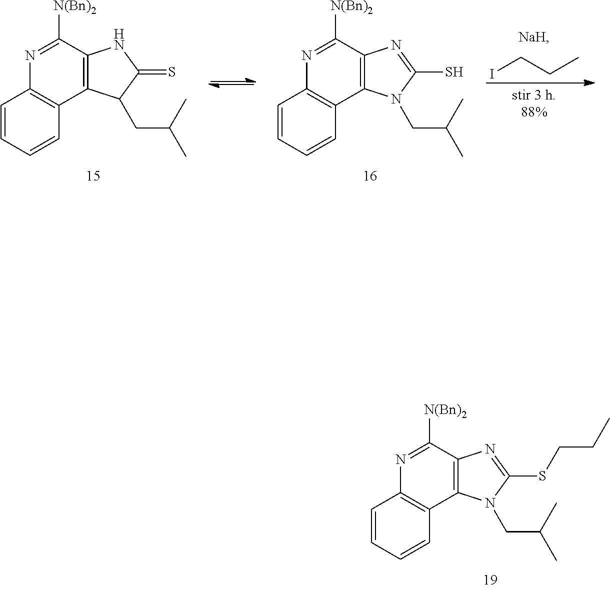

Alternatively, compound 1 (205 mg, 0.5 mmol, 1.0 eq.) is dissolved in dry methanol (20 mL), followed by addition of carbon disulfide (0.03 mL, 0.5 mmol, 1.0 eq.). After refluxing overnight, the solution is concentrated and then taken up in CH₂Cl₂. The mixture is washed with water, saturated NaHCO₃ and dried over sodium sulfate. To a solution of 15 (16) (226.3 mg, 0.5 mmol, 1.0 eq.) in THF 20 mL was added 60% sodium hydride (100 mg, 2.5 mmol, 5.0 eq.), followed by the addition of iodopropane (0.098 mL, 1.0 mmol, 2.0 eq.). The mixture was stirred under N₂ at room temperature for 3 hours. The solution was concentrated, and the residue was partitioned between ethyl acetate and water. The organic layer was washed with brine and dried. Concentration gave an oil residue which was chromatographed on a column of silica-gel. The column was eluted with a 20:1 mixture (v/v) hexane and ethyl acetate. Concentration of the combined fractions gave the product as a solid (224 mg, 88%).

19

¹H NMR (300 MHz, CDCl₃) 0.89 (t, J=7.2 Hz, 3H), 1.10 (d, J=6.6 Hz, 6H), 1.67 (m, 2H), 2.50 (m, 1H), 3.13 (t, J=7.2 Hz, 2H), 4.30 (d, J=7.5 Hz, 2H), 5.51 (s, 4H), 7.28-7.97 (m, 14H);

¹³C NMR (300 MHz, CDCl₃) 13.9, 20.6, 23.6, 29.8, 35.8, 51.3, 53.9, 115.4, 120.0, 122.0, 127.3, 127.4, 128.5, 128.7, 128.9, 129.3, 136.8, 140.6, 145.3, 149.4, 150.8; HRMS (EI) obsd 494.2501 (M⁺), calcd (M⁺) 494.2499 (C₃₁H₃₄N₄S₁)

Step III

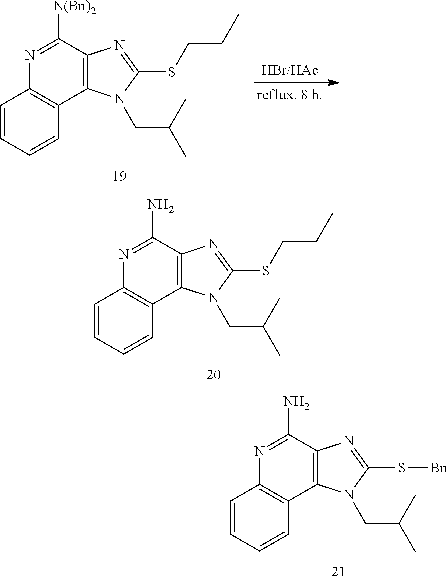

A solution of 19 (1 eq) in hydrogen bromide (10 mL, 47% in water) and acetic acid (10 mL) was refluxed for 8 hours. The reaction solution was diluted with CH₂Cl₂ (100 mL), and brought to pH 7 with a 1M NaOH solution and a saturated solution of NaHCO₃. The organic layer was separated, dried, and concentrated. The crude product was purified by chromatography using 5% methanol in dichloromethane sequentially, giving products 20 and 21 as a mixture, the mole ratio shown by ¹H-NMR is 2.0:2.8.

20

¹H NMR (300 MHz, CDCl₃) 1.00 (d, J=6.6 Hz, 6H), 1.05 (t, J=7.5 Hz, 3H), 1.80 (m, 2H), 2.27 (m, 1H), 3.32 (t, J=7.5 Hz, 2H), 4.25 (d, J=7.5 Hz, 2H), 5.65 (s, 2H), 7.24-7.84 (m, 4H); HRMS (EI) obsd 314.1556 (M⁺), calcd 314.1560 (M⁺) (C₁₇H₂₂N₄S₁)

21

¹H NMR (300 MHz, CDCl₃) 0.93 (d, J=6.6 Hz, 6H), 2.28 (m, 1H), 4.13 (d, J=7.8 Hz, 2H), 4.53 (s, 2H), 5.71 (s, 2H), 7.24-7.84 (m, 9H)

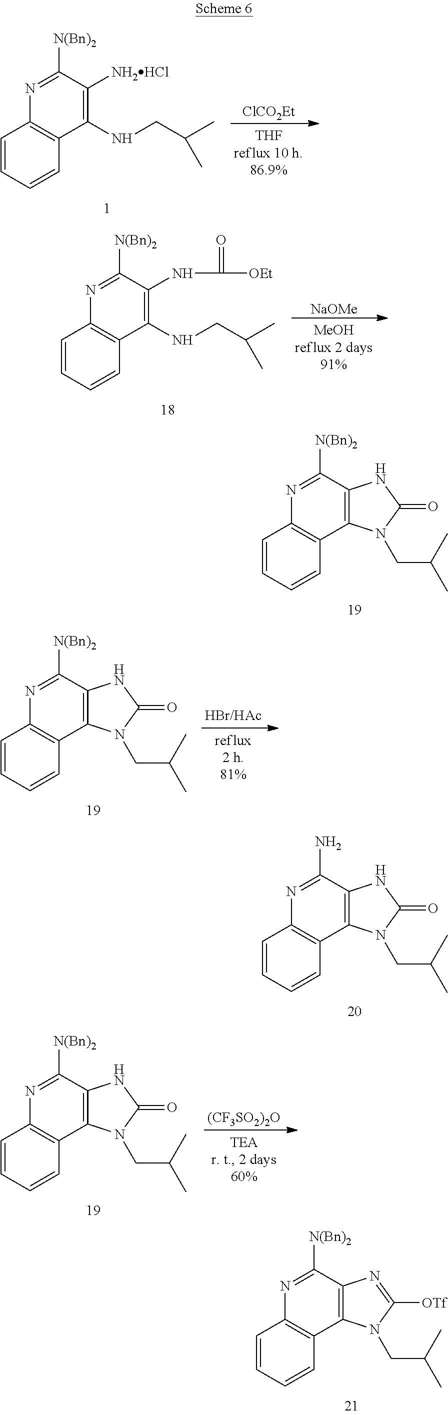

Step I

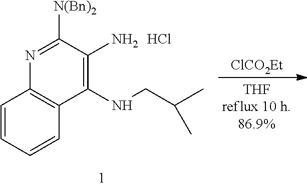

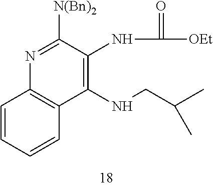

| Material | Sourced by: | MW | Qty. | | Mole | Ration |
|---|---|---|---|---|---|---|
| 1 | prepared | 447.01 | 134 | mg | 0.3 mmol | 1.0 eq. |
| ethyl | Merck | 108.5 | 0.14 | mL | 1.5 mmol | 5.0 eq. |
| chloroformate | | | | | | |
| THF | LAB-SCAN | | 20 | mL | | |

To a solution of 1 (134 mg, 0.3 mmol, 1.0 eq.) in THF (20 mL), was added ethyl chloroformate (0.15 mL, 1.4 mmol, 5.0 eq.). The mixture was refluxed under N₂ for 10 hours. The solution was concentrated, and the residue was partitioned between dichloromethane and water. The organic layer was dried and concentrated. The crude residue was chromatographed on a column of silica-gel. The column was eluted with an 8:1 mixture (v/v) of hexane and ethyl acetate. Concentration of the combined fractions gave the product 18 as solid (0.126 g, 86.9%).

18

mp 99.7-101.2° C.; ¹H NMR (300 MHz, CDCl₃) 0.91 (d, J=6.6 Hz, 6H), 1.15 (t, J=7.2 Hz, 3H), 1.74 (m, 1H), 3.24 (m, 2H), 4.10 (q, J=7.2 Hz, 2H), 4.41 (s, 4H), 4.71 (s, 1H), 6.47 (1H), 7.20-7.94 (m, 14H); HRMS (EI) obsd 482.2679 (M⁺), calcd 482.2676 (M⁺) (C₃₀H₃₄N₄O₂)

Step II

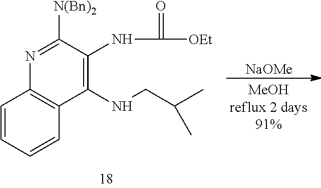

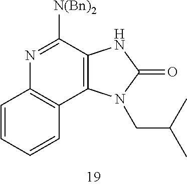

| Material | Sourced by: | MW | Qty. | | Mole | Ration |
|---|---|---|---|---|---|---|
| 18 | prepared | 482.6 | 483 | mg | 1.0 mmol | 1.0 eq. |
| NaOMe | Panreac | 54.0 | 108 | mg | 2 0 mmol | 2.0 eq. |
| methanol | LAB-SCAN | | 50 | mL | | |

To a solution of 18 (483 mg, 1.0 mmol, 1.0 eq.) in dry methanol (50 mL), was added NaOMe (108 mg, 2.0 mmol, 2.0 eq.). The mixture was refluxed under N₂ for 2 days. The solution was concentrated, and the residue was partitioned between dichloromethane and water. The organic layer was dried and concentrated. The residue was dry loaded on silica gel (1 g) and then chromatographed on a column of silica gel. The column was eluted with a 5:1 mixture (v/v) hexane and ethyl acetate. Concentration of the combined fractions gave the product as white solid (396 mg, 91%).

19

mp 210.1-211.9° C.; ¹H NMR (300 MHz, CDCl₃) 1.01 (d, J=6.6 Hz, 6H), 2.28 (m, 1H), 4.05 (d, J=7.5 Hz, 2H), 4.86 (s, 4H), 7.25-7.86 (m, 14H), 7.89 (s, 1H); ¹³C NMR (300 MHz, CDCl₃) 19.8, 28.8, 49.5, 51.6, 111.3, 113.6, 119.8, 123.0, 126.8, 127.1, 128.0, 128.4, 130.8, 138.2, 143.9, 146.5, 155.3; HRMS (EI) obsd 436.2258 (M⁺), calcd 436.2258 (M⁺) (C₂₈H₂₈N₄O₁)

Step III

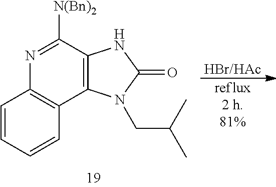

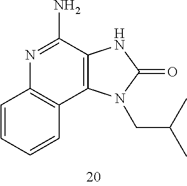 [SMILES: CC(C)Cn1c(=O)[nH]c2c(N)nc3ccccc3c21]

| | Sourced | | | | | |
|---|---|---|---|---|---|---|
| Material | by: | MW | Qty. | | Mole | Ration |
| 19 | prepared | 436.2 | 87.3 | mg | 0.2 mmol | 1.0 eq. |
| Hydrogen bromide | Merck | | 10 | mL | | |
| (47% in water) | | | | | | |
| Acetic acid | Fisher | | 10 | mL | | |

A solution of 19 (87.3 mg, 0.2 mmol, 1.0 eq.) in hydrogen bromide (10 mL, 47% in water) and acetic acid (10 mL) was refluxed for 2 hours. The reaction solution was diluted with CH₂Cl₂ (100 mL), and brought to pH 7 with a 1M NaOH solution and a saturated solution of NaHCO₃. The organic layer was separated, dried, and concentrated. The crude product was purified by chromatography using 10% methanol in dichloromethane. Yield: 81%

20

¹H NMR (300 MHz, MeOD) 1.00 (d, J=6.6 Hz, 6H), 2.22 (m, 1H), 4.11 (d, J=7.5 Hz, 2H), 7.31-7.98 (m, 4H); MS (EI) 256.

Step IV

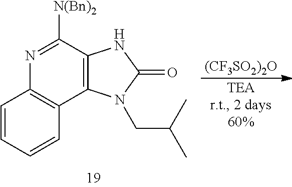

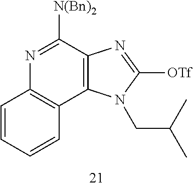

| Material | Sourced by: | MW | Qty. | | Mole | Ration |
|---|---|---|---|---|---|---|
| 19 | prepared | 436.5 | 1.757 | g | 4.0 mmol | 1.0 eq. |
| Triethylamine | Riedel-de | 101.2 | 0.67 | mL | 4.8 mmol | 1.2 eq. |
| | Haën | | | | | |
| (CF₃SO₂)₂O | Merck | 282.1 | 0.81 | mL | 4.8 mmol | 1.2 eq. |
| dichloromethane | LAB-SCAN | | 50 | mL | | |

To a solution of 19 (1.757 g, 4.0 mmol, 1.0 eq.) in dry dichloromethane (50 mL) was added triethylamine (0.67 mL, 4.8 mmol, 1.2 eq.) and trifluoromethanesulphonic anhydride (0.81 mL, 4.8 mmol, 1.2 eq.), sequentially. The mixture was stirred under N₂ for 2 days. The solution was partitioned between CH₂Cl₂ and water. The organic layer was dried and concentrated. The residue was chromatographed on a column of silica-gel. The column was eluted with 5% ethyl acetate in hexane. Concentration of the combined fractions gave the product as white solid (1.36 g, 60%).

21

mp 108.2-109.6° C.; ¹H NMR (300 MHz, CDCl₃) 1.06 (d, J=6.6 Hz, 6H), 2.40 (m, 1H), 4.25 (d, J=7.5 Hz, 2H), 5.29 (s, 4H), 7.21-7.84 (m, 14H); ¹³C NMR (300 MHz, CDCl₃) 20.4, 29.5, 51.2, 53.3, 114.5, 119.9, 122.7, 123.9, 127.5, 128.6, 128.7, 128.8, 129.0, 134.5, 139.9, 143.4, 146.1, 150.9; ¹⁹F NMR (400 MHz, CDCl₃)-13.42; HRMS (EI) obsd 568.1756 (M⁺), calcd 568.1750 (M⁺) (C₂₉H₂₇F₃N₄O₃S₁); Anal. Calcd for C₂₉H₂₇F₃N₄O₃St: C, 61.26; H, 4.79; N, 9.85. Found: C, 61.36; H, 4.78; N, 9.85.

As will be apparent to one skilled in the art, compound 21 is a very useful intermediate, that may be easily functionalized by displacement of the triflate with many substituents, including substituted amines, thiols, carbonyls, oxo and alkoxy groups, and alkenyl and alkynyl moieties, among others.

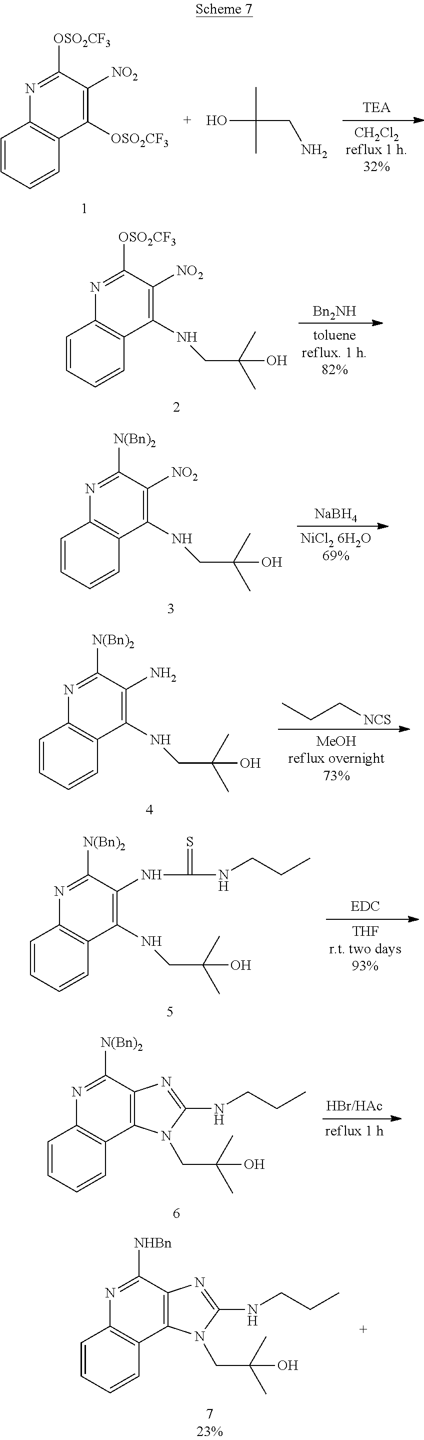

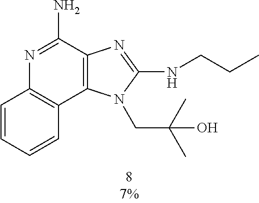 [SMILES: CCCNc1nc2c(N)nc3ccccc3c2n1CC(C)(C)O]

Step I

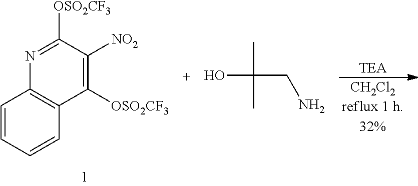

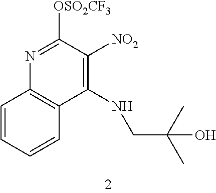

| Material | Sourced by: | MW | Qty. | | Mole | Ration |
|---|---|---|---|---|---|---|
| 1 | prepared | 470.3 | 470 | mg | 1 mmol | 1.0 eq. |
| 1-amino-2- | Prepared¹ | 89.1 | 89 | mg | 1 mmol | 1.0 eq. |
| methyl- | | | | | | |
| 2-propanol | | | | | | |
| TEA | Aldrich | 101.2 | 0.14 | mL | 1 mmol | 1.0 eq. |
| CH₂Cl₂ | LAB-SCAN | | 30 | mL | | |

For reference see Close, W. J., Abbott Labs., North Chicago, J. Amer. Chem. Soc., 1951, 73, 95-8.

To a solution of 1 (470 mg, 1 mmol, 1.0 eq.) in dry CH₂Cl₂ (30 mL), was added triethylamine (0.14 mL, 1 mmol, 1.0 eq.), followed by the addition of 1-amino-2-methyl-2-propanol (89 mg, 1 mmol, 1.0 eq.). The mixture was refluxed for 1 hour under a blanket of N₂. The reaction solution was partitioned between dichloromethane and water. The organic layer was dried and concentrated to give an oil residue which was chromatographed on a column of silica gel. The column was eluted with a 5:4 mixture (v/v) hexane and ethyl acetate. Concentration of the yellow fractions gave the product as a solid (132 mg, 32%).

2

mp 139.6-139.8° C.; ¹H NMR (300 MHz, CDCl₃) 1.34 (s, 6H), 3.50 (d, J=5.1 Hz, 2H), 7.57-8.08 (m, 4H), 7.89 (s, 1H); HRMS (EI) obsd 409.0553 (M⁺), calcd 409.0550 (M⁺) (C₁₄H₁₄F₃N₃O₆S₁)

Step II

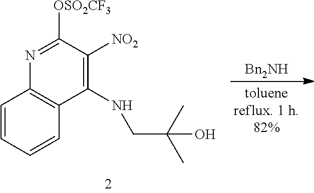

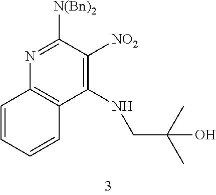

| Material | Sourced by: | MW | Qty. | | Mole | Ration |
|---|---|---|---|---|---|---|
| 2 | prepared | 409.3 | 8.066 | g | 0.020 mol | 1.0 eq. |
| Dibenzyl amine | Aldrich | 197.2 | 5.8 | mL | 0.03 mol | 1.5 eq. |
| toluene | LAB-SCAN | | 250 | mL | | |

To a solution of 2 (8.066 g, 0.020 mol, 1.0 eq.) in dry toluene (250 mL) was added dibenzylamine (5.8 mL, 0.03 mol, 1.5 eq.). The mixture was refluxed for 1 hour under N₂. The mixture was concentrated and the residue was chromatographed on a column of silica gel. The column was eluted with a 5:1 mixture (v/v) hexane and ethyl acetate. Concentration of the red fractions gave the product as a solid (7.4 g, 82%).

3

¹H NMR (300 MHz, CDCl₃) 1.24 (s, 6H), 3.63 (d, J=5.1 Hz, 2H), 4.53 (s, 4H), 7.17-7.97 (m, 15H); ¹³C NMR (300 MHz, CDCl₃) 28.0, 53.2, 60.0, 71.2, 116.9, 122.2, 126.7, 127.7, 128.4, 128.8, 129.0, 129.1, 132.6, 128.1, 149.3, 151.2, 153.3; HRMS (EI) obsd 456.2156 (M⁺), calcd 456.2156 (M⁺) (C₂₇H₂₈N₄O₃)

Step III

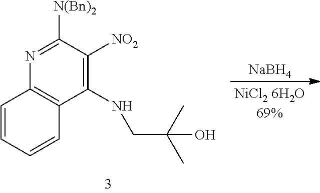

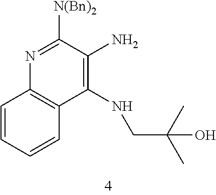

| Material | Sourced by: | MW | Qty. | Mole | Ration |
|---|---|---|---|---|---|
| NiCl₂ 6H₂O | Merck | 237.7 | 89.2 mg | 0.375 | 0.75 eq. |
| | | | | mmol | |
| NaBH₄ | Aldrich | 37.8 | 28.4 mg + | 0.75 | 1.5 eq. + |
| | | | 75.6 mg | mmol + | 4.0 eq. |
| | | | | 2.0 mmol | |
| 3 | prepared | 456.5 | 228 mg | 0.5 mmol | 1.0 eq. |
| DCM | LAB-SCAN | | 10 mL | | |
| methanol | LAB-SCAN | | 20 mL | | |

To a 100 mL three necked round bottom flask fitted with guard tube was added methanol (10 mL) and nickel chloride (89.2 mg, 0.375 mmol, 0.75 eq.). Subsequently, NaBH₄ (28.4 mg, 0.75 mmol, 1.5 eq.) was added in small lots while maintaining the temperature at 25° C. The solution was stirred for 30 minutes and then 3 (228 mg, 0.5 mmol, 1.0 eq.) in DCM (10 mL) and methanol (10 mL) was added. NaBH₄ (75.6 mg, 2.0 mmol, 4.0 eq.) was added in small lots while maintaining the temperature at 35° C. A colorless solution with a black precipitate was observed. The reaction solution was filtered through Celite brand filter aid, and the filtrate was concentrated and adsorbed and chromatographed on a column of silica-gel. The column was eluted with a 10:3 mixture (v/v) hexane and ethyl acetate. Concentration of the fractions gave the product as an oil (146 mg, 69%).

4

¹H NMR (300 MHz, CDCl₃) 1.23 (s, 6H), 3.05 (s, 2H), 4.14 (s, 2H), 4.41 (s, 4H), 7.10-7.71 (m, 14H); ¹³C NMR (300 MHz, CDCl₃) 28.1, 54.0, 57.5, 71.9, 119.7, 122.9, 124.7, 126.0, 127.2, 127.5, 128.9, 129.1, 135.2, 139.6, 142.3, 153.9; HRMS (EI) obsd 426.2421 (M⁺), calcd 426.2414 (M⁺) (C₂₇H₃₀N₄O₁)

Step IV

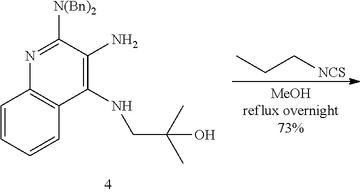

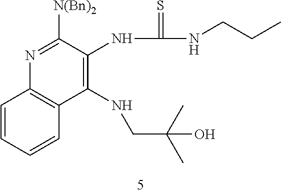

| Material | Sourced by: | MW | Qty. | | Mole | Ration |
|---|---|---|---|---|---|---|
| 4 | prepared | 426.5 | 146 | mg | 0.34 mmol | 1.0 eq. |
| propyl | Acros | 101.17 | 0.042 | mL | 0.41 mmol | 1.2 eq. |
| isothiocyanate | | | | | | |
| methanol | LAB-SCAN | | 15 | mL | | |

Compound 4 (146 mg, 0.34 mmol, 1.0 eq.) was dissolved in dry methanol (15 mL), and propyl isothiocyanate (0.042 mL, 0.41 mmol, 1.2 eq.) was added. After refluxing overnight under N₂, the solution was concentrated, and the residue was partitioned between CH₂Cl₂ and water. The organic layer was dried and concentrated. The crude residue was chromatographed on a column of silica gel. The column was eluted with a 10:3 mixture (v/v) hexane and ethyl acetate. Concentration of the combined fractions gave the product as an oil (131 mg, 73%).

5

¹H NMR (300 MHz, CDCl₃) 0.67 (t, J=7.5 Hz, 3H), 1.13 (s, 6H), 1.27 (m, 2H), 2.27 (s, 1H), 3.31 (m, 4H), 4.58 (s, 4H), 5.14 (1H), 5.65 (1H), 7.07-7.75 (m, 15H); HRMS (EI) obsd 527.2719 (M⁺), calcd 527.2713 (M⁺) (C₃₁H₃₇N₅O₁S₁)

Step V

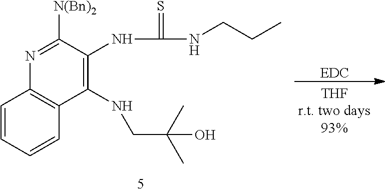

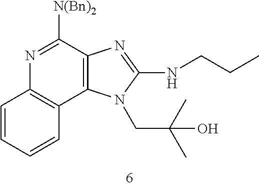

| Material | Sourced by: | MW | Qty. | | Mole | Ration |
|---|---|---|---|---|---|---|
| 5 | prepared | 527.7 | 131 | mg | 0.25 mmol | 1.0 eq. |
| EDC | Acros | 191.7 | 95 | mg | 0.5 mmol | 2.0 eq. |
| THF | LAB-SCAN | | 15 | mL | | |

To a solution of 5 (131 mg, 0.25 mmol, 1.0 eq.) in dry THF (15 mL), was added EDC (95 mg, 0.5 mmol, 2.0 eq.). The reaction solution was stirred for two days under N₂. The mixture was then concentrated, and the residue was partitioned between CH₂Cl₂ and water. The organic layer was washed with a saturated solution of sodium chloride, dried, and concentrated. The crude residue was chromatographed on a column of silica gel. The column was eluted with a 5:1 mixture (v/v) hexane and ethyl acetate. Concentration of the combined fractions gave the product as an oil (115 mg, 84%).

6

¹H NMR (300 MHz, CDCl₃) 0.79 (t, J=7.5 Hz, 3H), 1.31 (s, 6H), 1.51 (m, 2H), 1.93 (1H), 3.16 (m, 2H), 4.25 (s, 2H), 5.31 (s, 4H), 5.83 (1H), 7.04-7.74 (m, 14H); ¹³C NMR (300 MHz, CDCl₃) 11.5, 22.7, 27.7, 45.1, 50.3, 54.3, 73.8, 114.7, 117.8, 120.7, 125.2, 126.1, 126.4, 127.9, 128.1, 128.2, 133.5, 140.2, 143.8, 150.1, 153.7; HRMS (EI) obsd 493.2832 (M⁺), calcd 493.2836 (M⁺) (C₃₁H₃₅N₅O₁)

Step VI

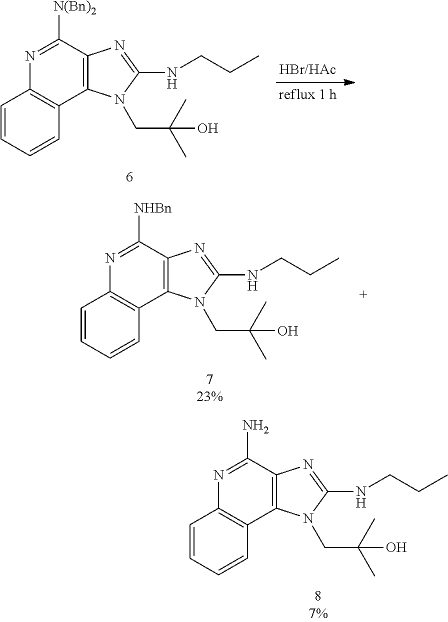

A solution of 6 (115 mg, 0.23 mmol, 1.0 eq.) in hydrogen bromide (10 mL, 47% in water) and acetic acid (10 mL) was refluxed for 1 hour. The reaction solution was diluted with CH₂Cl₂ (100 mL), and brought to pH 7 with a 1M NaOH solution and a saturated solution of NaHCO₃. The organic layer was separated, dried, and concentrated. Four products were shown from TLC. Products 7 and 8 were purified by chromatography using 2.5%, 10% methanol in CH₂Cl₂, respectively.

7

¹H NMR (300 MHz, CDCl₃) 0.90 (t, J=7.5 Hz, 3H), 1.34 (s, 6H), 1.58 (m, 2H), 3.28 (m, 2H), 4.25 (s, 2H), 4.88 (d, J=5.7 Hz, 2H), 5.91 (1H), 5.99 (1H), 7.11-7.82 (m, 9H); HRMS (EI) obsd 403.2355 (M⁺), calcd 403.2367 (M⁺) (C₂₄H₂₉N₅O₁)

8

¹H NMR (300 MHz, CDCl₃) 0.94 (t, J=7.5 Hz, 3H), 1.40 (s, 6H), 1.65 (m, 2H), 3.35 (m, 2H), 4.31 (s, 2H), 5.42 (s, 2H), 6.09 (t, 1H), 7.14-7.78 (m, 4H); HRMS (EI) obsd 313.1889 (M⁺), calcd 313.1897 (M⁺) (C₁₇H₂₃N₅O₁)

Step VII

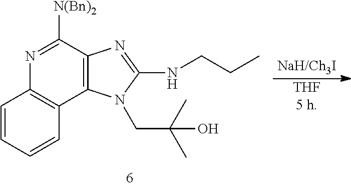

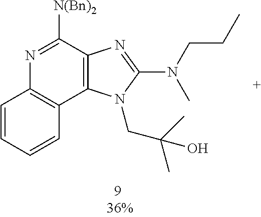

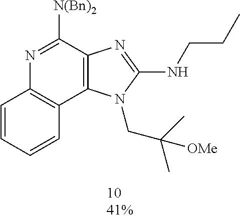

| Material | Sourced by: | MW | Qty. | | Mole | Ration |
|---|---|---|---|---|---|---|
| 6 | prepared | 493.6 | 2.014 | g | 4.1 mmol | 1.0 eq. |
| 60% sodium | Panreac | 24.0 | 163 | mg | 4.1 mmol | 1.0 eq. |
| hydride | | | | | | |
| iodomethane | Acros | 141.94 | 0.25 | mL | 4.1 mmol | 1.0 eq. |
| THF | LAB-SCAN | | 40 | mL | | |

To a solution of 6 (2.014 g, 4.1 mmol, 1.0 eq.) in THF (40 mL) was added 60% sodium hydride (163 mg, 4.1 mmol, 1.0 eq.), followed by addition of iodomethane (0.25 mL, 4.1 mmol, 1.0 eq.). The mixture was stirred under N₂ for 5 hours. The reaction solution was concentrated, and the residue was partitioned between ethyl acetate and water. The organic layer was washed with brine, and dried. Concentration gave an oil residue which was chromatographed on a column of silica gel. The column was eluted with a 15:1 mixture (v/v) hexane and ethyl acetate. Concentration of the combined fractions gave the product 10 as a solid (41%) and the product 9 as an oil (36%).

9

¹H NMR (300 MHz, CDCl₃) 0.85 (t, J=7.2 Hz, 3H), 1.20 (s, 6H), 1.53 (m, 2H), 2.78 (s, 3H), 2.98 (t, J=7.8 Hz, 2H), 4.59 (s, 1H), 4.66 (s, 2H), 5.38 (s, 4H), 7.22-8.04 (m, 14H); ¹³C NMR (300 MHz, CDCl₃) 12.2, 20.8, 28.2, 41.5, 51.2, 56.3, 58.6, 73.1, 115.6, 119.7, 121.8, 126.0, 127.2, 128.6, 128.7, 128.8, 135.0, 140.5, 145.6, 151.1, 156.0; HRMS (EI) obsd 507.2988 (M⁺), calcd 507.2993 (M⁺) (C₃₂H₃₇N₅O₁)

10

¹H NMR (300 MHz, CDCl₃) 0.92 (t, J=7.5 Hz, 3H), 1.37 (s, 6H), 1.63 (m, 2H), 3.26 (s, 3H), 3.30 (m, 2H), 4.40 (s, 2H), 5.41 (s, 4H), 6.08 (t, J=5.4 Hz, 1H), 7.18-7.86 (m, 14H); ¹³C NMR (300 MHz, CDCl₃) 11.5, 21.3, 22.7, 44.9, 49.1, 50.3, 55.2, 77.9, 114.8, 117.7, 120.7, 125.1, 126.2, 126.4, 128.1, 128.3, 133.5, 140.3, 143.8, 150.1, 153.9; HRMS (EI) obsd 507.2990 (M⁺), calcd 507.2993 (M⁺) (C₃₂H₃₇N₅O₁)

Step VIII

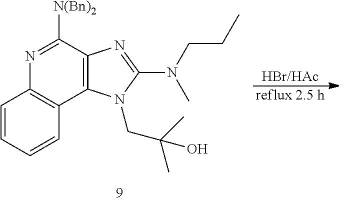

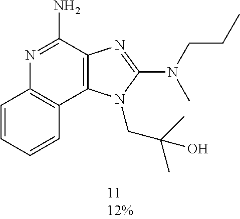 [SMILES: CCCN(C)c1nc2c(N)nc3ccccc3c2n1CC(C)(C)O]

| | Sourced | | | | | |
|---|---|---|---|---|---|---|
| Material | by: | MW | Qty. | | Mole | Ration |
| 34 | prepared | 507.7 | 203 | mg | 0.4 mmol | 1.0 eq. |
| Hydrogen | Merck | | 10 | mL | | |
| bromide | | | | | | |
| (47% in water) | | | | | | |
| Acetic acid | Fisher | | 10 | mL | | |

A solution of 9 (203 mg, 0.4 mmol, 1.0 eq.) in hydrogen bromide (10 mL, 47% in water) and acetic acid (10 mL) was refluxed for 2.5 hours. The reaction solution was diluted with CH₂Cl₂ (100 mL) and brought to pH 7 with a 1M NaOH solution and a saturated solution of NaHCO₃. The organic layer was separated, dried, and concentrated. The product (11) was purified by chromatography using 4% methanol in dichloromethane.

11

¹H NMR (300 MHz, CDCl₃) 0.96 (t, J=7.5 Hz, 3H), 1.20 (s, 6H), 1.67 (m, 2H), 2.87 (s, 3H), 3.07 (t, J=7.8 Hz, 2H), 4.09 (s, 1H), 4.58 (s, 2H), 5.92 (s, 2H), 7.25-8.10 (m, 4H); HRMS (EI) obsd 327.2054 (M⁺), calcd 327.2054 (M⁺) (C₁₈H₂₅N₅O₁)

Step VIIa (Alternative Method)

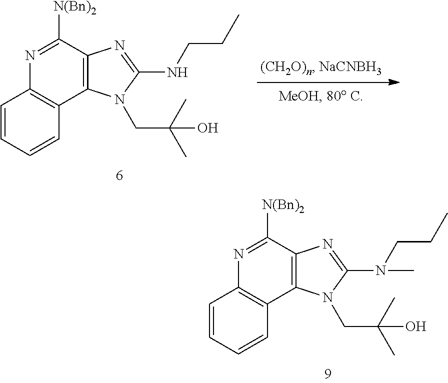

A mixture of 6 (1 eq) and paraformaldehyde (5 eq) is dissolved in a solution of MeOH and AcOH (5:1) on molecular sieves. NaCNBH₃ (4 eq.) is added to the suspension at 25° C. The slurry is subsequently heated to 80° C. After 10 hours, the mixture is cooled, filtered, and concentrated. The residue is dissolved in CH₂Cl₂ and washed with saturated NaHCO₃. The organic solution is dried (Na₂SO₄), and concentrated to yield 9. Subsequent debenzylation according to Step VIII, yields the final product (11).

The following Schemes describe methods of producing preferred 2-alkenyl or 2-alkynyl imidazo[4,5-c]quinolin-4-amine derivatives. It will be apparent to one skilled in the art that reagents and/or substituents can be altered or substituted to optimize or further functionalize the compounds described below.

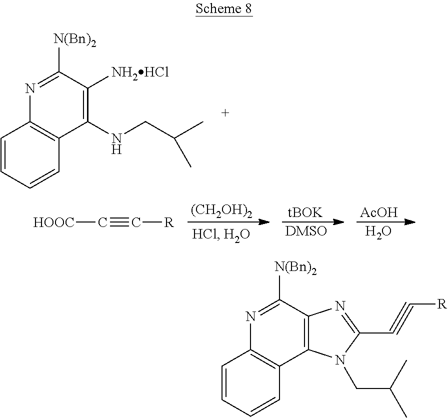

wherein, the R group may be H, alkyl, or aryl, preferably phenyl.

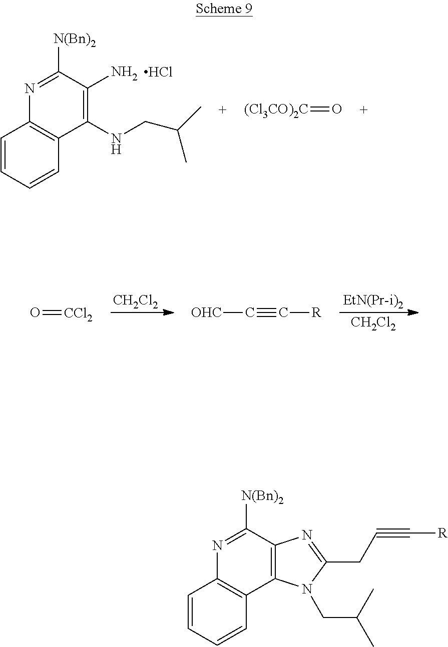

wherein, the R group may be H, alkyl, or aryl, preferably phenyl.

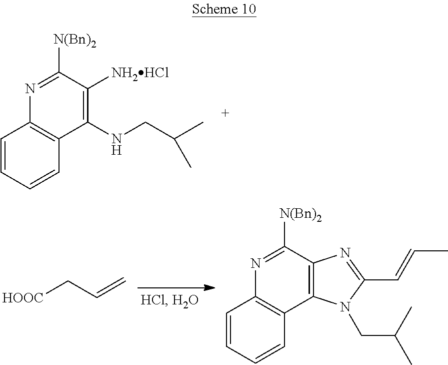

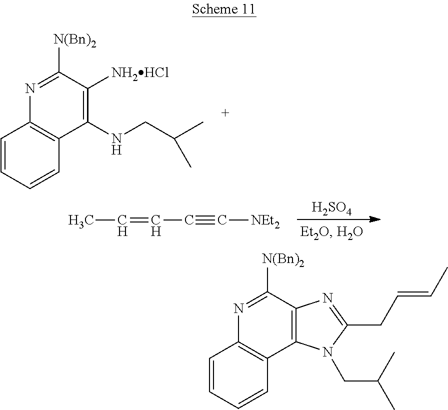

Alternatively, triflic acid may be used in place of H₂SO₄.

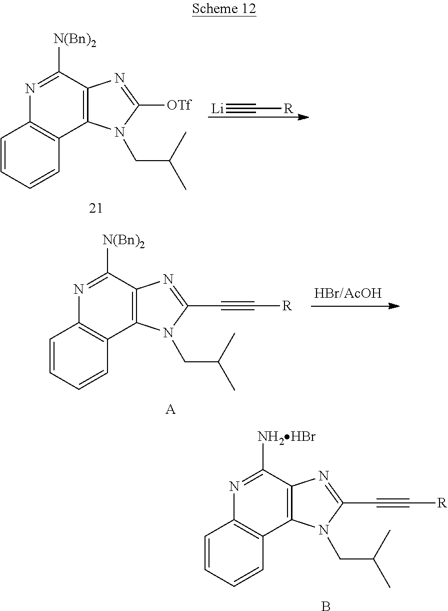

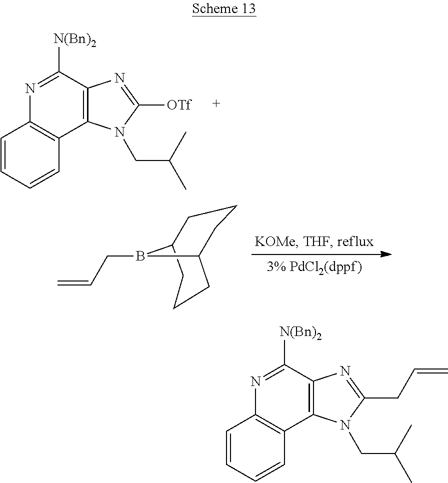

It is also contemplated that conversion of the triflate substituent in the reactant to a halide, such as bromo, and subsequent coupling with an alkenyl or alkynyl moiety having at least three carbon atoms in Et₃N with CuI, Ph₃P and Pd(OAc)₂, will yield many of the products described in Schemes 8-13.

Where the reaction of any of Schemes 8-13 fails to proceed in full, heat may be added to facilitate completion.

Subsequent debenzylation of the products in Schemes 8-13 to provide a free amine at the 4-position, is performed in boiling MeCN using NaI and TMSCl to afford TMSI in situ. Removal of the resultant TMS functionality is performed in THF with Bu₄N⁺F⁻. Alternatively, depending on the stability of the alkenyl or alkynyl substituent bound at position 2 of the N-TMS-imidazo[4,5-c]quinolin-4-amine derivatives, K₂CO₃, citric acid, HF, or polystyrene sulfonic acid in MeOH may be used to cleave the resultant TMS group. Alternatively, the debenzylation may proceed as described previously in the presence of HBr and acetic acid (as shown in Scheme 11).

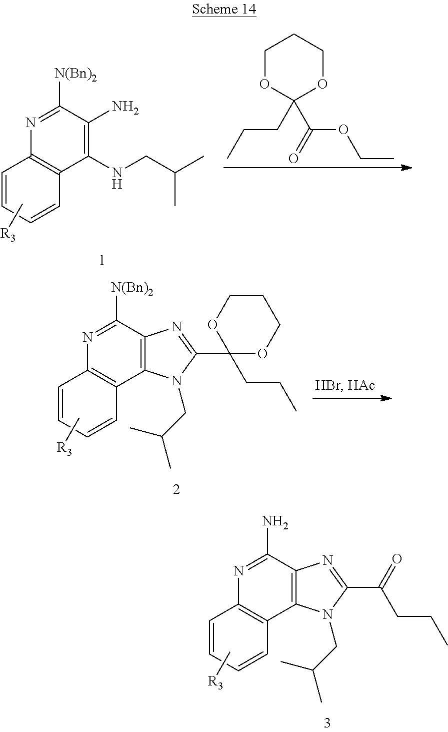

Cyclization of 1 to form the imidazoquinoline analog of 2 is performed by addition of the ketal and heat in THF. Once complete, the reaction is condensed, washed with water, and extracted into CHCl₃. The mixture is then dried over sodium sulfate and purified by silica gel chromatography. Debenzylation with hydrogen bromide and acetic acid is then performed as described previously (with heat), which also results in hydrolysis of the ketal to the desired ketone. Silica gel chromatography yields the product (3).

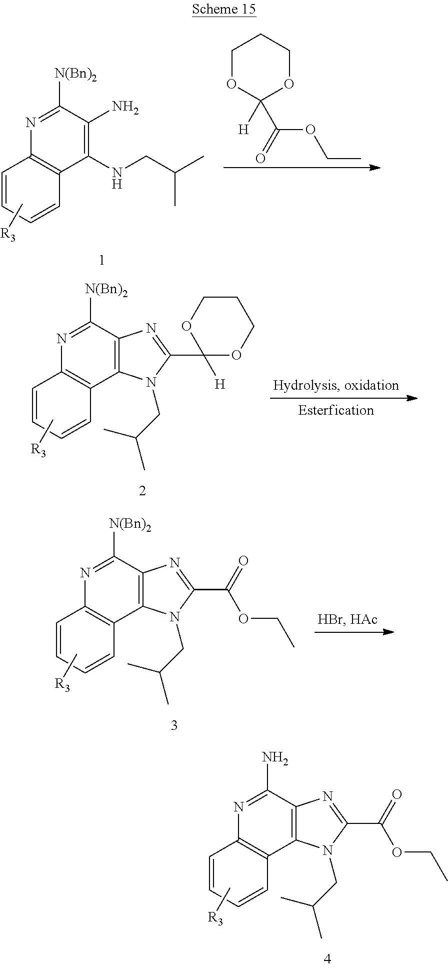

Cyclization of 1 to form the imidazoquinoline analog of 2 is performed by addition of the acetal and heat in THF. Once complete, the reaction is condensed, washed with water, and extracted into CHCl₃. Hydrolysis of the acetal with aqueous HCl is followed by a Swern Oxidation to yield the carboxylic acid. Finally, esterification is performed in the presence of HCl and an alcohol such as ethanol. Debenzylation with hydrogen bromide is then performed as described previously to afford the final product (4). Alternatively, the debenzylation may be performed as described for Schemes 8-13 above.

TABLE 1
Imidazoquinoline Compounds

| Example | Structure | Name |
|---|---|---|
| 1 | 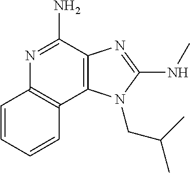 [SMILES: CNc1nc2c(N)nc3ccccc3c2n1CC(C)C] | N2-methyl-1-(2-methylpropyl)-1H- imidazo[4,5-c]quinoline-2,4-diamine |
| 2 | 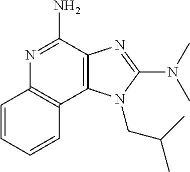 [SMILES: CC(C)Cn1c(N(C)C)nc2c(N)nc3ccccc3c21] | N2,N2-dimethyl-1-(2-methylpropyl)-1H- imidazo[4,5-c]quinoline-2,4-diamine |
| 3 | 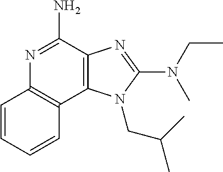 [SMILES: CCN(C)c1nc2c(N)nc3ccccc3c2n1CC(C)C] | N2-ethyl-N2-methyl-1-(2-methylpropyl)- 1H-imidazo[4,5-c]quinoline-2,4-diamine |
| 4 | 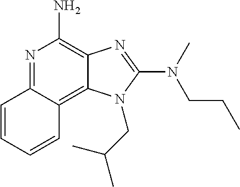 [SMILES: CCCN(C)c1nc2c(N)nc3ccccc3c2n1CC(C)C] | N2-methyl-1-(2-methylpropyl)-N2-propyl- 1H-imidazo[4,5-c]quinoline-2,4-diamine |
| 5 | 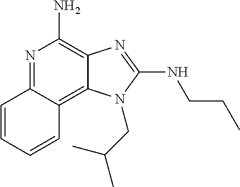 [SMILES: CCCNc1nc2c(N)nc3ccccc3c2n1CC(C)C] | 1-(2-methylpropyl)-N2-propyl-1H- imidazo[4,5-c]quinoline-2,4-diamine |
| 6 | 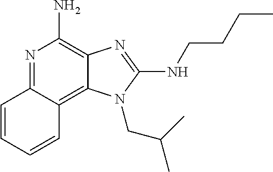 [SMILES: CCCCNc1nc2c(N)nc3ccccc3c2n1CC(C)C] | N2-butyl-1-(2-methylpropyl)-1H- imidazo[4,5-c]quinoline-2,4-diamine |
| 7 | 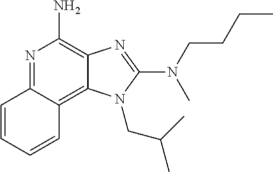 [SMILES: CCCCN(C)c1nc2c(N)nc3ccccc3c2n1CC(C)C] | N2-butyl-N2-methyl-1-(2-methylpropyl)- 1H-imidazo[4,5-c]quinoline-2,4-diamine |
| 8 | 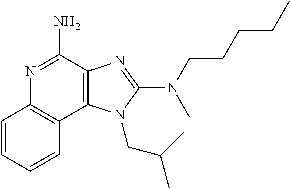 [SMILES: CCCCCN(C)c1nc2c(N)nc3ccccc3c2n1CC(C)C] | N2-methyl-1-(2-methylpropyl)-N2-pentyl- 1H-imidazo[4,5-c]quinoline-2,4-diamine |
| 9 | 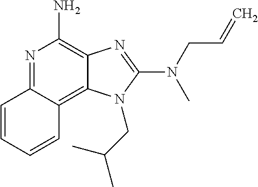 [SMILES: C=CCN(C)c1nc2c(N)nc3ccccc3c2n1CC(C)C] | N2-methyl-1-(2-methylpropyl)-N2-prop-2- enyl-1H-imidazo[4,5-c]quinoline-2,4- diamine |
| 10 | 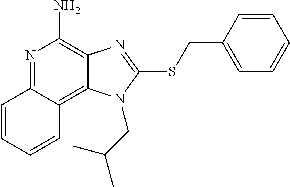 [SMILES: CC(C)Cn1c(SCc2ccccc2)nc2c(N)nc3ccccc3c21] | 1-(2-methylpropyl)-2-[(phenylmethyl)thio]- 1H-imidazo[4,5-c]quinolin-4-amine |
| 11 | 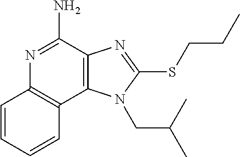 [SMILES: CCCSc1nc2c(N)nc3ccccc3c2n1CC(C)C] | 1-(2-methylpropyl)-2-(propylthio)-1H- imidazo[4,5-c]quinolin-4-amine |
| 12 | 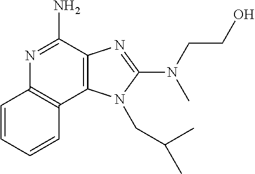 [SMILES: CC(C)Cn1c(N(C)CCO)nc2c(N)nc3ccccc3c21] | 2-[[4-amino-1-(2-methylpropyl)-1H- imidazo[4,5-c]quinolin-2- yl](methyl)amino]ethanol |
| 13 | 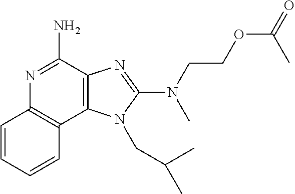 [SMILES: CC(=O)OCCN(C)c1nc2c(N)nc3ccccc3c2n1CC(C)C] | 2-[[4-amino-1-(2-methylpropyl)-1H- imidazo[4,5-c]quinolin-2- yl](methyl)amino]ethyl acetate |
| 14 | 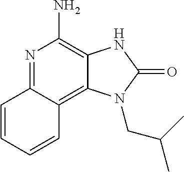 [SMILES: CC(C)Cn1c(=O)[nH]c2c(N)nc3ccccc3c21] | 4-amino-1-(2-methylpropyl)-1,3-dihydro- 2H-imidazo[4,5-c]quinolin-2-one |
| 15 | 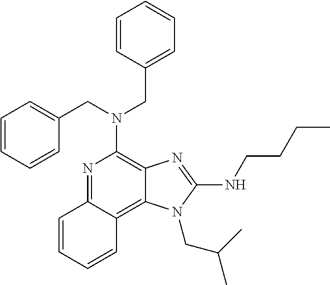 [SMILES: CCCCNc1nc2c(N(Cc3ccccc3)Cc3ccccc3)nc3ccccc3c2n1CC(C)C] | N2-butyl-1-(2-methylpropyl)-N4,N4- bis(phenylmethyl)-1H-imidazo[4,5- c]quinoline-2,4-diamine |
| 16 | 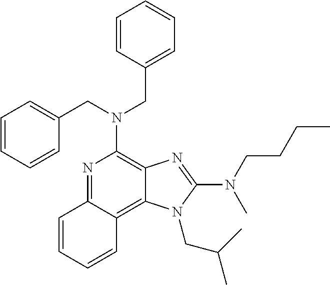 [SMILES: CCCCN(C)c1nc2c(N(Cc3ccccc3)Cc3ccccc3)nc3ccccc3c2n1CC(C)C] | N2-butyl-N2-methyl-1-(2-methylpropyl)- N4,N4-bis(phenylmethyl)-1H-imidazo[4,5- c]quinoline-2,4-diamine |
| 17 | 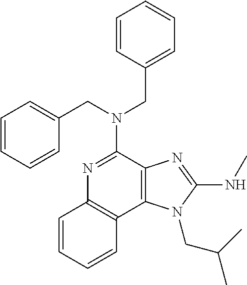 [SMILES: CNc1nc2c(N(Cc3ccccc3)Cc3ccccc3)nc3ccccc3c2n1CC(C)C] | N2-methyl-1-(2-methylpropyl)-N4,N4- bis(phenylmethyl)-1H-imidazo[4,5- c]quinoline-2,4-diamine |
| 18 | 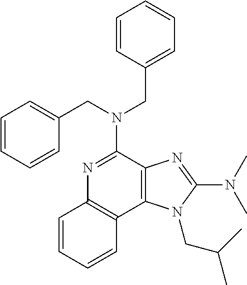 [SMILES: CC(C)Cn1c(N(C)C)nc2c(N(Cc3ccccc3)Cc3ccccc3)nc3ccccc3c21] | N2,N2-dimethyl-1-(2-methylpropyl)- N4,N4-bis(phenylmethyl)-1H-imidazo[4,5- c]quinoline-2,4-diamine |
| 19 | 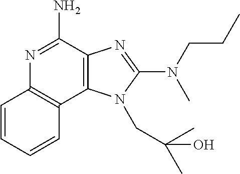 [SMILES: CCCN(C)c1nc2c(N)nc3ccccc3c2n1CC(C)(C)O] | 1-{4-amino-2-[methyl(propyl)amino]-1H- imidazo[4,5-c]quinolin-1-yl}-2- methylpropan-2-ol |
| 20 | 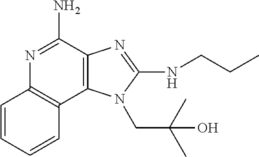 [SMILES: CCCNc1nc2c(N)nc3ccccc3c2n1CC(C)(C)O] | 1-[4-amino-2-(propylamino)-1H- imidazo[4,5-c]quinolin-1-yl]-2- methylpropan-2-ol |
| 21 | 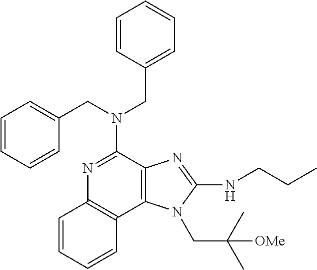 [SMILES: CCCNc1nc2c(N(Cc3ccccc3)Cc3ccccc3)nc3ccccc3c2n1CC(C)(C)OC] | N4,N4-dibenzyl-1-(2-methoxy-2- methylpropyl)-N2-propyl-1H-imidazo[4,5- c]quinoline-2,4-diamine |
| 22 | 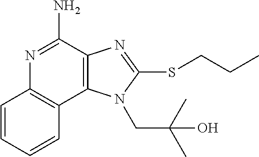 [SMILES: CCCSc1nc2c(N)nc3ccccc3c2n1CC(C)(C)O] | 1-(4-Amino-2-propylsulfanyl-imidazo[4,5- c]quinolin-1-yl)-2-methyl-propan-2-ol |
| 23 | 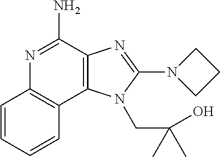 [SMILES: CC(C)(O)Cn1c(N2CCC2)nc2c(N)nc3ccccc3c21] | 1-(4-Amino-2-azetidin-1-yl-imidazo[4,5- c]quinolin-1-yl)-2-methyl-propan-2-ol |
| 24 | 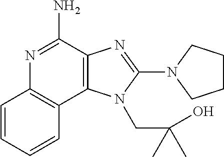 [SMILES: CC(C)(O)Cn1c(N2CCCC2)nc2c(N)nc3ccccc3c21] | 1-(4-Amino-2-pyrrolidin-1-yl-imidazo[4,5- c]quinolin-1-yl)-2-methyl-propan-2-ol |
| 25 | 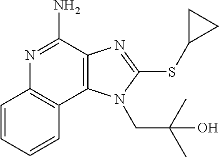 [SMILES: CC(C)(O)Cn1c(SC2CC2)nc2c(N)nc3ccccc3c21] | 1-(4-Amino-2-cyclopropylsulfanyl- imidazo[4,5-c]quinolin-1-yl)-2-methyl- propan-2-ol |
| 26 | 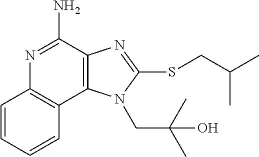 [SMILES: CC(C)CSc1nc2c(N)nc3ccccc3c2n1CC(C)(C)O] | 1-(4-Amino-2-isobutylsulfanyl- imidazo[4,5-c]quinolin-1-yl)-2-methyl- propan-2-ol |

Each of the Example compounds 1-21 listed in Table 1 were synthesized according to the Schemes listed above. Many of the Example compounds were screened in the assay described below for their ability to induce cytokines. Many of these compounds showed activity at less than 5 μM with respect to production of TNF-α. Some of these compounds showed activity in the production of TNF-α at less than 1.5 μM. Further, some of these compounds showed activity in the production of TLR-7 and/or TLR-8. For this reason, each of the R groups of any of the compounds listed in Table 1 is preferred. Additionally, because of the excellent activity of the compounds, each of these compounds is individually preferred and is preferred as a member of a group that includes any or all of the other compounds and each compound is preferred in methods of modulating an immune response and in methods of treating biological conditions associated therewith, for example to be used as a vaccine adjuvant. Each of the compounds is also preferred for use in preparation of medicaments for immunopotentiation, reducing tumor growth, treating microbial and viral infections, particularly HCV and HSV, and in treating biological conditions mediated therefrom.

Several Example compounds were screened and found to not be effective at a concentration of 20 μM or less using the assay described below, the majority of those being protected intermediates of the final compounds. These compounds are also useful within the scope of the invention, since the invention is not meant to be limited to those compounds that are useful at a concentration of 20 μM or less. Compounds may be useful as intermediates, or as final products that cause production of TNF-α at higher concentrations, such as 100 μM, 200 μM or 300 M in the assays described herein. For example Loxoribine causes useful production of TNF-α at 300 μM (see Pope et al. Cellular Immunology 162: 333-339 (1995)).

### Biological Assays

Candidate small molecule immunopotentiators can be identified in vitro. Compounds are screened in vitro for their ability to activate immune cells. One marker of such activation is the induction of cytokine production, for example TNF-α production. Apoptosis inducing small molecules may be identified having this activity. These small molecule immuno-potentiators have potential utility as adjuvants and immuno-therapeutics.

In an assay procedure (High Throughput Screening (HTS)) for imidazoquinoline small molecule immune potentiators (SMIPs), human peripheral blood mononuclear cells (PBMC), 500,000 per mL in RPMI 1640 medium with 10% FCS, are distributed in 96 well plates (100,000 per well) already containing 5 μM of compound in DMSO. The PBMCs are incubated for 18 hours at 37° C. in 5% CO₂. Their ability to produce cytokines in response to the small molecule compounds is determined using a modified sandwich ELISA.

Briefly, supernatants from the PBMC cultures are assayed for secreted TNF using a primary plate bound antibody for capture followed by a secondary biotinylated anti-TNF antibody forming a sandwich. The biotinylated second antibody is then detected using streptavidin-europium, and the amount of bound europium is determined by time resolved fluorescence. Imidazoquinoline compounds are confirmed by their TNF inducing activity that is measured in the assay as increased europium counts over cells incubated in RPMI medium alone. “Hits” are selected based on their TNF-inducing activity relative to an optimal dose of lipopolysaccharide LPS (1 μg/mL), a strong TNF inducer. The robustness of the assay and low backgrounds allowed for the routine selection of hits with ˜10% of LPS activity that is normally between 5-10× background (cells alone). Selected hits are then subjected to confirmation for their ability to induce cytokines from multiple donors at decreasing concentrations. Those compounds with consistent activity at or below 5 μM are considered confirmed for the purposes of this assay. The assay is readily modified for screening for compounds effective at higher or lower concentrations.

In addition to the procedure described above, methods of measuring other cytokines (e.g., IL1-beta, IL-12, IL-6, IFN-gamma, IL-10 etc.) are well known in the art and can be used to find active imidazoquinoline compounds of the present invention.

Qualitative and quantitative measurement of the immune response of a SMIP or composition comprising a SMIP of the preferred embodiments of the present invention can be implemented using methods known in the art, such as by measuring antigen specific antibody production, activation of specific populations of lymphocytes such as CD4⁺, CD8+ T cells or NK cells, and/or production of cytokines such as IFN, IL-2, IL-4 or IL-12. Methods for measuring specific antibody responses include enzyme-linked immunosorbent assay (ELISA) as known in the art. Measurement of numbers of specific types of lymphocytes such as CD4⁺ T cells can be achieved, for example, with fluorescence-activated cell sorting (FACS). Cytotoxicity assays can also be performed using methods known in the art, e.g., as described in Raz et al., (1994) Proc. Natl. Acad. Sci. USA 91:9519-9523. Serum concentrations of cytokines can be measured, for example, by ELISA. Such assays are described, e.g., in Selected Methods in Cellular Immunology (1980) Mishell and Shiigi, eds., W.H. Freeman and Co.

### Additional Biological Methods

### I. Sample Preparations

### Human PBMC Preparation

Human blood from one or multiple human donors were collected into the BD Vacutainer™ CPT tube with sodium citrate (BD, Franklin Lakes, N.J.), and spun for 20 minutes at 1600 g. After centrifugation, mononuclear cells in the top layer in the tubes were collected and then washed three times with PBS buffer. The washed cells were then reconstituted at a required cell concentration in complete RPMI containing 10% FBS plus 100 units/ml penicillin and 100 ug/ml streptomycin.

### Mouse Spleen Cell Preparation

Spleens were isolated from Balbc mice and minced to release the splenocytes from the tissues. After the minced samples were treated ammonium salt to destroy the red blood cells, the rest of the spleenocytes were washed and reconstituted at a required cell concentration with completed RPMI medium.

### Human THP-1 Cell Line

The human myelomonocytic transformed cell line is responsive to TLR8 agonists and weakly to TLR7 agonists. The cell line is cultured in RPMI medium supplemented with 10% FBS.

### II. Activity Measurement

### Compound Stimulation and Multi-Cytokine Measurement

Human PBMC (hPBMC) (at 1 million cells/ml) or mouse spleen cells (at 5 million cells/ml) or human monocytic THP-1 cells (at 1 million cells/ml) were mixed with tested compounds such as imidazoqualines at titrated compound concentrations in the complete RPMI medium. After the cell cultures were incubated for 24 hours at 37° C., 5% CO₂, the culture supernatant was collected and assayed for the secreted cytokines in the presence of the compounds. Human or mouse Beadlyte multi-cytokine flex kits (Upstate, Lake Placid, N.Y.) were used to measure the amount of the following cytokines: TNF-α, IL-6, IL-1β, IL-8 and IL-12p40 according to the manufacturers instructions.

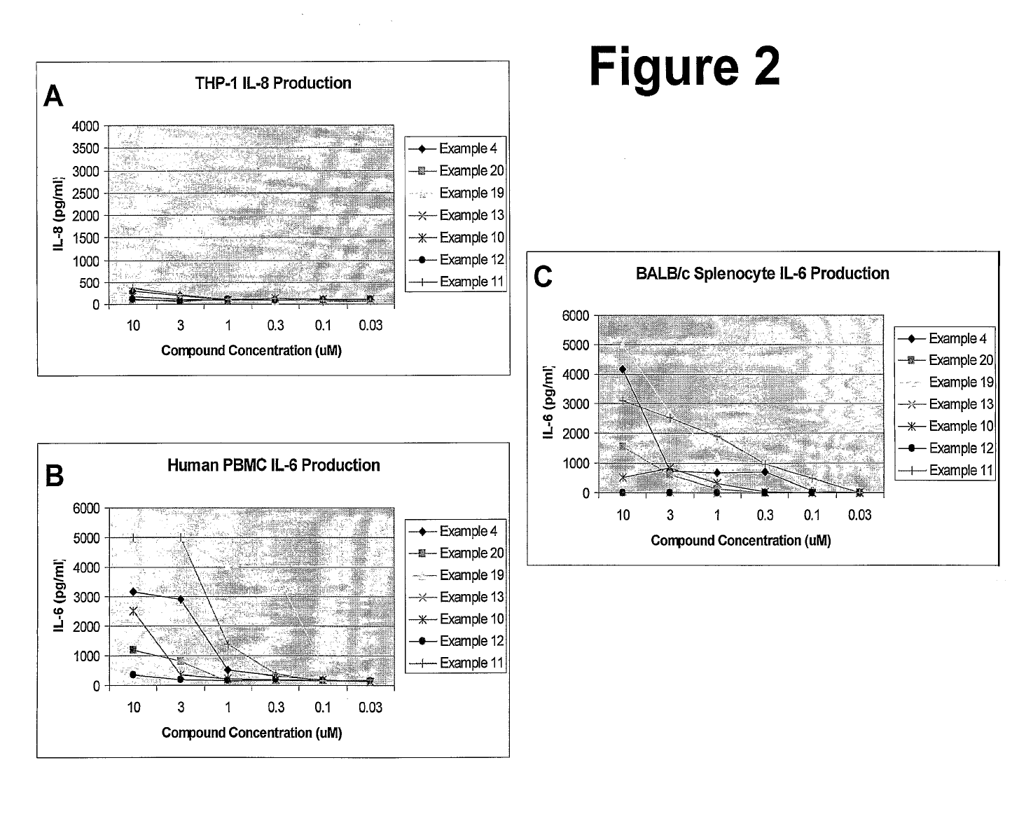

FIGS. 2A-C show results for the myelomonocytic cell line, THP-1 (FIG. 2A), human PBMC (FIG. 2BB) and murine splenocytes (FIG. 2C) capacity to produce cytokines in response to decreasing doses of the compounds of Examples 4, 20, 19, 13, 10, 12 and 11. For each cell population multiple cytokines were assayed (e.g. IL-12, IFN-g, IL-1b, IL-10, TNF-α etc.) and the levels of human IL-8 (A); human IL-6 (B) and murine IL-6 (C) are shown.

### TLR Signaling

HEK293 cells (ATCC, CRL-1573) were seeded in a T75 flask at 3×10⁶ in 20 ml of DMEM supplemented with 0.1 mM nonessential amino acid, 1 mM sodium pyruvate, 2 mM L-glutamine, penicillin-streptomycin, and 10% FCS. After overnight culturing, the cells were transfected with 1) pNFkB-TA-luciferase reporter (0.4 ug) (BD clontech, Palo Alto, Calif.), and with 2) with pGL4.74 (0.01 ug) that carries a TK promoter, not responsive to NF-kB stimulation, and carries a Renilla luciferase gene, used as an internal control (Promega, Wis.), and 3), separately with a following TLR construct (10 ug): human TLR (hTLR) 7, hTLR8, mouse TLR7 (mTLR7) puno constructs (Invivogene, Calif.), using Fugene 6 transfection reagent (Roche). The transfected cells after 24 hours transfection were collected and seeded in a 96-well and flat-bottom plate (1×10⁴ cell/well) plate, and stimulated with the test compounds at the following concentrations: 30, 10, 3, 1, 0.3, 0.1, 0.03 uM. After overnight compound stimulation, the cells were assayed for expression of fly and renilla luciferases using Dual-Luciferase Reporter Assay System (Promega, Wis.). NF-kb activation is directly proportional to relative fly luciferase units, which is measured against the internal control renilla luciferase units.

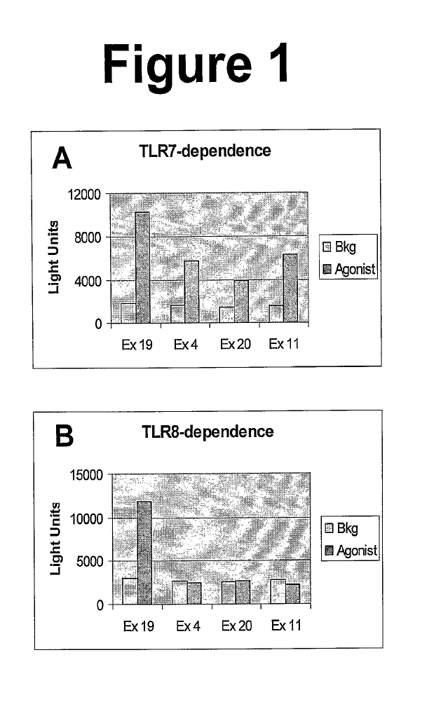

FIG. 1 shows the results for TLR7-dependence (FIG. 1A) and TLR8-dependence (FIG. 1B) of SMIPS of Examples 19, 4, 20 and 11, using the 20 μM dose. The negative controls were TLR7 or 8 transfected HEK293-NFkB-luciferase cells in medium alone, and these results were similar to those obtained using untransfected (TLR7 or 8) HEK293-NFkB-luciferase expressing cells.

### Standardization of Cytokine Production

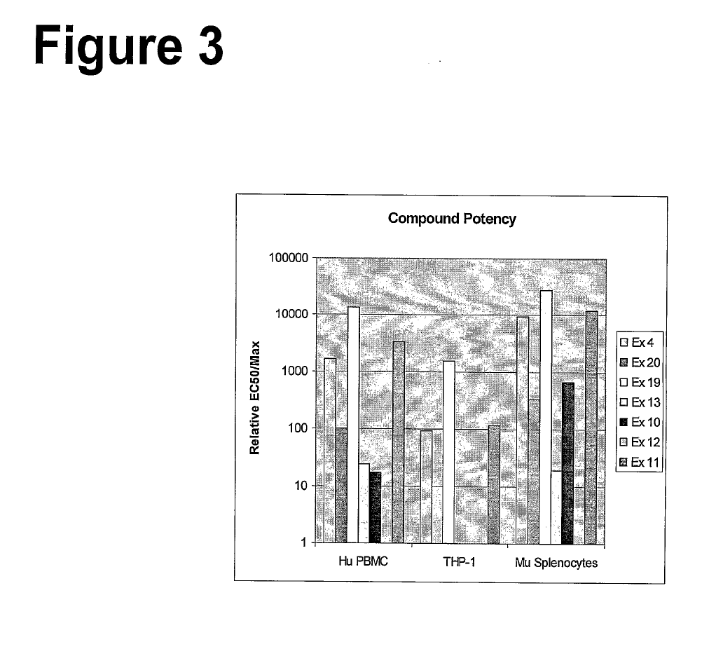

Due to the agonist nature of the compounds tested, compound ranking is based on potency in cell-based screens for cytokine induction. Briefly, the EC₅₀ of each compound for a given cytokine is calculated relative to a reference composition (i.e. LPS). This value is then used as the divisor of the maximum level of cytokine produced (pg/ml) in the assay. FIG. 3 shows the ranking of SMIP potency in varying cell lines. Five parameter curve fitting of cytokine dose response curves to different SMIPs for the indicated cell populations is used to calculate EC50. Rank-scoring of SMIP potency is calculated by dividing the maximum concentration of cytokine produced by the relative EC50 established for each compound indicated. For human THP-1 cells IL-8 induction was used for the calculation, for human PBMC, IL-6 and for murine splenocytes, IL-6.

### In Vivo Adjuvant Studies

In phosphate-buffered saline (PBS), 25 micrograms gp120dV2EnvSF162 antigen (recombinant gp120 protein derived from sequence of HIV-1 strain SF162—the V2 domain was deleted; Pharm Res. 2004 Dec. 21(12):2148-52) as mixed with 50 microliters of MF59 adjuvant, followed the by the addition of 0, 1, 5, or 25 micrograms of a small molecule immune potentiator (SMIP) and adjusted to 100 microliters with PBS. 50 microliters of the solution was subsequently injected into both the left and right tibialis anterior muscles of female BALB/c mice (Day 0), for a total volume of 100 microliters per mouse. Four weeks later (Day 28), 50 microliters of the solution was again injected into both the left and right tibialis anterior muscles of the mouse. Seven days after the second vaccination (Day 34), serum samples were collected, and a day later (day 35) spleens were removed. Serum samples were assayed by Env-specific serum IgG2a ELISA and Env-specific serum IgG1 ELISA. Spleen samples were assayed by Env-specific, cytokine-producing splenic CD4 and CD8 T cells. The results are shown in Table 2.

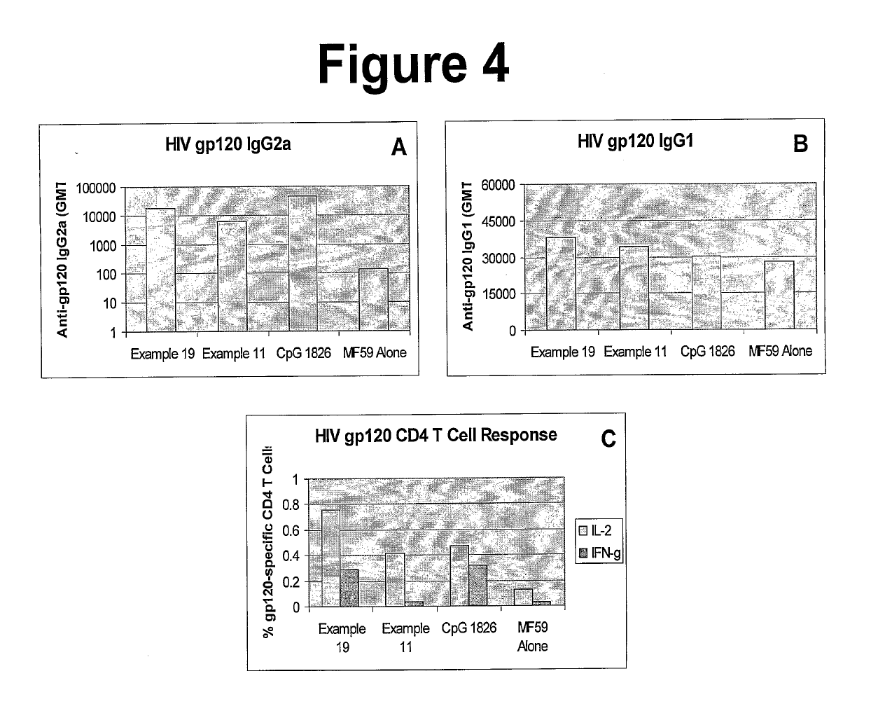

FIG. 4 shows in vivo adjuvant activity of the compounds of Example 19 and Example 11. BALB/c mise were immunized 2× with HIV gp120 formulated in MF59 +/− the indicated SMIPs (25 μg/ml). CpG 1826 (25 μg/ml) was used as a positive control. 2 weeks post-second serum was collected from the immunized mice and the anti-gp120-specific serum IgG2a (FIG. 4A) and IgG1 (FIG. 4B) geometric mean titers (GMT) were determined. In addition, spleens were also harvested from the immunized mice and ex vivo anti-gp120-specific T cell responses (FIG. 4C) were determined by intracellular cytokine staining for IL-2 and IFN-g. Results are percentage of antigen-specific T cells expressing the indicated cytokine.

TABLE 2
Summary of titers and T cell frequencies

| Added to gp120dV2/MF59 | | | | | | | | | |
|---|---|---|---|---|---|---|---|---|---|
| base vaccine^{a} | | Env-spec IgG2a titer | | Env-spec IgG1 titer | | Env-spec CD4 (%) | | Env-spec CD8 (%) | |
| Compound | Amt (μg) | Geo mean | range | Geo mean | range | IFN-γ+ | IL-2+ | IFN-γ+ | IL2+ |
| Example 19^{b} | 25 | 18733 | 8657-29097 | 38255 | 29232-45440 | 0.29 | 0.76 | 0.07 | 0.10 |
| | 5 | 10104 | 5751-16624 | 29502 | 15660-60635 | 0.04 | 0.40 | 0.35 | 0.37 |
| | 1 | 1215 | <250-6151 | 30536 | 17139-59828 | 0.07 | 0.14 | 0.21 | 0.14 |
| Example 11^{b} | 25 | 6507 | 2643-14319 | 34199 | 27057-42384 | 0.04 | 0.42 | 0.18 | 0.22 |
| | 5 | 2922 | 878-19357 | 33477 | 22697-60509 | 0.04 | 0.42 | 0.19 | 0.22 |
| | 1 | 512 | <250-1773 | 35177 | 28421-40356 | 0.04 | 0.17 | 0.08 | 0.17 |
| ODN-1826^{c} | 25 | 201306 | 158869-264363 | 30188 | 19678-47384 | 0.32 | 0.47 | 0.15 | 0.12 |
| DMSO^{d} | | 141 | <250-1125 | 27844 | 26857-28455 | 0.03 | 0.13 | 0.05 | 0.07 |

^{a}mice vaccinated day 0 and 28, sera and spleens collected 6-7 d after 2^{nd} vaccination
^{b}5 BALB/c
^{c}5 BALB/c: ODN-1826 = synthetic phosphorothioate oligodeoxynucleotide that contains unmethylated CpG motifs and has the sequence 5′-TCC ATG ACG TTC CTG ACG TT-3′
^{d}3 BALB/c: With MF59, no SMIP

We claim:

1. A compound of formula (I): 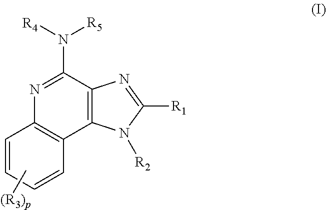
wherein:
R₁ is —NR₆R₇, —C(O)R₈, —C(O)OR₈, —C(O)NR₆R₇, —(CH₂)ₘCH═CH(CH₂)ₙR₉, —(CH₂)ₘC≡C(CH₂)ₙR₉, or —S(O)_{q}R₁₀;
R₂ is H, C₁₋₆ alkyl, substituted C₁₋₆ alkyl, —(CH₂)ₘCH═CH(CH₂)ₙR₉, —(CH₂)ₘC≡C(CH₂)ₙR₉, —C(O)R₈, —C(O)OR₈, —C(O)NR₆R₇, or —S(O)_{q}R₁₀;
each R₃ is independently H, C₁₋₆ alkyl, substituted C₁₋₆ alkyl, C₁₋₆ alkoxy, halogen, trihalomethyl, —NR₆R₇, —C(O)R₈, —C(O)OR₈, or —C(O)NR₆R₇;
R₄ and R₅ are each independently H, C₁₋₆ alkyl, C₆₋₁₀ aryl-C₁₋₆ alkyl, or a protecting group;
each R₆ and R₇ is independently H, C₁₋₆ alkyl, substituted C₁₋₆ alkyl, C₁₋₆ alkoxy, C₁₋₆ alkoxy-C₁₋₆ alkyl, C₆₋₁₀ aryl, C₆₋₁₀ aryl-C₁₋₆ alkyl, C₆₋₁₀ aryloxy-C₁₋₆ alkyl, —(CH₂)ₘCH═CH(CH₂)ₙR₉, or —(CH₂)ₘC≡C(CH₂)ₙR₉; or
R₆ and R₇ are taken together to form a substituted or unsubstituted heterocyclyl group;
each R₈ is independently H, C₁₋₆ alkyl or substituted C₁₋₆ alkyl;
each R₉ is independently H, C₁₋₆ alkyl, substituted C₁₋₆ alkyl, C₂₋₆ alkenyl, C₆₋₁₀ aryl, —CO₂H, —C(O)O—C₁₋₆ alkyl, or halo;
each R₁₀ is independently C₁₋₆ alkyl, substituted C₁₋₆ alkyl, C₂₋₆ alkenyl, C₆₋₁₀ aryl, C₆₋₁₀ aryl-C₁₋₆ alkyl, trihalomethyl, or —NR₆R₇;
each m and n is independently 0, 1, 2, or 3;
p is 0, 1, 2 or 3; and
each q is independently 0, 1 or 2; or
a pharmaceutically acceptable salt thereof, a tautomer thereof, or a pharmaceutically acceptable salt of the tautomer;
provided that if R₁ is —S-Me, then R₂ is not isobutyl.

2. The compound of claim 1, wherein R₄ and R₅ are each H.

3. The compound of claim 2, wherein R₁ is —NR₆R₇.

4. The compound of claim 2, wherein R₁ is —S(O)_{q}R₁₀.

5. The compound of claim 2, wherein R₁ is —C(O)NR₆R₇.

6. The compound of claim 2, wherein R₁ is —(CH₂)ₘCH═CH(CH₂)ₙR₉.

7. The compound of claim 2, wherein R₁ is —(CH₂)ₘC≡C(CH₂)ₙR₉.

8. The compound of claim 2, wherein R₂ is C₁₋₆ alkyl.

9. The compound of claim 3, wherein R₆ and R₇ within R₁ are independently H, C₁₋₆ alkyl or —(CH₂)ₘCH═CH(CH₂)ₙR₉.

10. The compound of claim 4, wherein R₁ is —SR₁₀, and the R₁₀ of the —SR₁₀ is C₁₋₆ alkyl.

11. The compound of claim 8, wherein R₂ is isobutyl.

12. The compound of claim 10, wherein the C₁₋₆ alkyl within R₁₀ is selected from methyl, ethyl, n-butyl, or n-pentyl.

13. The compound of claim 9, wherein m is 1, n is 0, and R₉ is H.

14. The compound of claim 2, wherein R₁ is —N(CH₃)CH₂CH₂CH₃.

15. The compound of claim 2, wherein p is 0.

16. The compound of claim 2, wherein R₂ is substituted C₁₋₆ alkyl.

17. The compound of claim 16, wherein R₂ is —CH₂C(CH₃)₂(OH).

18. The compound of claim 2, wherein R₁ is —S-cyclopropyl, —S—CH₂CH(CH₃)₂ or —S—CH₂CH₂CH₃.

19. The compound of claim 1, wherein R₁ is —S—C₃₋₆cycloalkyl.

20. The compound of claim 1, wherein R₆ and R₇ are taken together to form a substituted or unsubstituted heterocyclyl group.

21. The compound of claim 20, wherein said heterocyclyl group is selected from piperidinyl, pyrrolidinyl, azetidinyl, or aziridinyl.

22. The compound of claim 1, wherein the compound is selected from:
1-(4-Amino-2-propylsulfanyl-imidazo[4,5-c]quinolin-1-yl)-2-methyl-propan-2-ol;
1-(4-Amino-2-azetidin-1-yl-imidazo[4,5-c]quinolin-1-yl)-2-methyl-propan-2-ol;
1-(4-Amino-2-pyrrolidin-1-yl-imidazo[4,5-c]quinolin-1-yl)-2-methyl-propan-2-ol;
1-(4-Amino-2-cyclopropylsulfanyl-imidazo[4,5-c]quinolin-1-yl)-2-methyl-propan-2-ol; or
1-(4-Amino-2-isobutylsulfanyl-imidazo[4,5-c]quinolin-1-yl)-2-methyl-propan-2-ol;
or a pharmaceutically acceptable salt thereof

23. The compound of claim 1, wherein the compound is selected from:
N2-methyl-1-(2-methylpropyl)-1H-imidazo[4,5-c]quinoline-2,4-diamine;
N2,N2-dimethyl-1-(2-methylpropyl)-1H-imidazo[4,5-c]quinoline-2,4-diamine;
N2-ethyl-N2-methyl-1-(2-methylpropyl)-1H-imidazo[4,5-c]quinoline-2,4-diamine;
N2-methyl-1-(2-methylpropyl)-N2-propyl-1H-imidazo[4,5-c]quinoline-2,4-diamine;
1-(2-methylpropyl)-N2-propyl-1H-imidazo[4,5-c]quinoline-2,4-diamine;
N2-butyl-1-(2-methylpropyl)-1H-imidazo[4,5-c]quinoline-2,4-diamine;
N2-butyl-N2-methyl-1-(2-methylpropyl)-1H-imidazo[4,5-c]quinoline-2,4-diamine;
N2-methyl-1-(2-methylpropyl)-N2-pentyl-1H-imidazo[4,5-c]quinoline-2,4-diamine;
N2-methyl-1-(2-methylpropyl)-N2-prop-2-enyl-1H-imidazo[4,5-c]quinoline-2,4-diamine;
1-(2-methylpropyl)-2-[(phenylmethyl)thio]-1H-imidazo[4,5-c]quinolin-4-amine;
1-(2-methylpropyl)-2-(propylthio)-1H-imidazo[4,5-c]quinolin-4-amine;
2-[[4-amino-1-(2-methylpropyl)-1H-imidazo[4,5-c]quinolin-2-yl](methyl)amino]ethanol;
2-[[4-amino-1-(2-methylpropyl)-1H-imidazo[4,5-c]quinolin-2-yl](methyl)amino]ethyl acetate;
4-amino-1-(2-methylpropyl)-1,3-dihydro-2H-imidazo[4,5-c]quinolin-2-one;
N2-butyl-1-(2-methylpropyl)-N4,N4-bis(phenylmethyl)-1H-imidazo[4,5-c]quinoline-2,4-diamine;
N2-butyl-N2-methyl-1-(2-methylpropyl)-N4,N4-bis(phenylmethyl)-1H-imidazo[4,5-c]quinoline-2,4-diamine;
N2-methyl-1-(2-methylpropyl)-N4,N4-bis(phenylmethyl)-1H-imidazo[4,5-c]quinoline-2,4-diamine;
N2,N2-dimethyl-1-(2-methylpropyl)-N4,N4-bis(phenylmethyl)-1H-imidazo[4,5-c]quinoline-2,4-diamine;
1-{4-amino-2-[methyl(propyl)amino]-1H-imidazo[4,5-c]quinolin-1-yl}-2-methylpropan-2-ol;
1-[4-amino-2-(propylamino)-1H-imidazo[4,5-c]quinolin-1-yl]-2-methylpropan-2-ol; or
N4,N4-dibenzyl-1-(2-methoxy-2-methylpropyl)-N2-propyl-1H-imidazo[4,5-c]quinoline-2,4-diamine;
or a pharmaceutically acceptable salt thereof.

24. A pharmaceutical composition, comprising: the compound of claim 2 and a pharmaceutically acceptable excipient.
